(19) United States
(12) Patent Application Publication
(10) Pub. No.: US 2024/0327817 A1
(43) Pub. Date: Oct. 3, 2024

(54) TOOL AND METHOD FOR DISAGGREGATION OF POLYQ STRETCH-CONTAINING PROTEINS

(71) Applicant: Universität zu Köln, Köln (DE)

(72) Inventors: Ernesto LLAMAS PAMANES, Köln (DE); David VILCHEZ GUERRERO, Köln (DE); Alga ZUCCARO, Köln (DE)

(73) Assignee: Universität zu Köln, Köln (DE)

(21) Appl. No.: 18/586,191

(22) Filed: Feb. 23, 2024

Related U.S. Application Data
(60) Provisional application No. 63/541,314, filed on Sep. 29, 2023.
(60) Provisional application No. 63/447,903, filed on Feb. 24, 2023.

(51) Int. Cl. C12N 9/50; A61K 38/00; C12N 9/64

(52) U.S. Cl. CPC C12N 9/63; C12N 9/6489; C12Y 304/21102; C12Y 304/24056; C12Y 304/24061; C12Y 304/24064; A61K 38/00

(57) ABSTRACT

The present invention provides an isolated protein exhibiting an antiaggregating activity and/or disaggregating activity toward a target protein comprising an extended polyQ stretch. The protein comprises a Zn²⁺-binding region, wherein the conserved motif is HxxEHx₇₅₋₈₀E and x is any amino acid. The nucleic acid construct encoding said protein as well as the corresponding mRNA sequence are also provided. The protein, the nucleic acid construct or mRNA sequence are for use in a method for prevention or treatment of a neurodegenerative disease that is caused by aggregates comprising at least one target protein and/or by the mRNA encoding for said target protein, wherein the target protein causes e.g. Huntington's disease or Machado-Joseph disease.

### CROSS REFERENCE TO RELATED APPLICATIONS

The present application claims priority under 35 USC 119(e) to U.S. Provisional Application No. 63/541,314, filed Sep. 29, 2023, and U.S. Provisional Application No. 63/447,903, filed Feb. 24, 2023, which applications are incorporated by reference herein in their entireties.

### REFERENCE TO SEQUENCE LISTING SUBMITTED ELECTRONICALLY

The instant application contains a Sequence Listing which has been submitted herewith in the form of an XML file. The Sequence Listing, created on Feb. 22, 2024, and named P70607.xml, is 115 KB in size, and is herein incorporated by reference in its entirety.

### BACKGROUND

The present invention provides an isolated protein exhibiting an antiaggregating activity and/or disaggregating activity toward a target protein comprising an extended polyQ stretch. The protein comprises a Zn²⁺-binding region, wherein the conserved motif is HxxEHx₇₅₋₈₀E and x is any amino acid. The nucleic acid construct encoding said protein as well as the corresponding mRNA sequence are also provided. The protein, the nucleic acid construct or mRNA sequence are for use in a method for prevention or treatment of a neurodegenerative disease that is caused by aggregates comprising at least one target protein and/or by the mRNA encoding for said target protein, wherein the target protein causes e.g. Huntington's disease or Machado-Joseph disease.

Proteins containing polyglutamine repeats (polyQ) are prone to aggregation and can lead to distinct human pathologies. In humans, aggregation of polyglutamine repeat (polyQ) proteins causes disorders such as Huntington's disease. For instance, Huntington's disease is caused by an abnormal expansion of the polyQ stretch (>Q35) of Huntingtin (HTT) protein (Ross et al. 2004; Perutz 1999; Orr 2001). However, plants express hundreds of proteins containing polyQ regions (Kottenhagen et al. 2012), but no pathologies arising from these factors have been reported to date. The isolated chloroplast stromal processing peptidase (SPP) from Arabidopsis thaliana suppresses aggregation of target proteins comprising an extended polyQ stretch (>Q35) in human cells. It is shown that expression of SPP reduces neuronal Q67 aggregation and subsequent neurotoxicity.

Across the proteome, numerous proteins are prone to self-assembly into pathological aggregates¹. Many human neurodegenerative diseases involve proteins with prion-like domains or intrinsically disordered regions rich in asparagine (N) and glutamine (Q) residues, which promote aggregation2. For instance, a common feature of polyQ-containing proteins is their capacity to form aggregates in yeast and higher eukaryotes3. However, cells have evolved proteostasis mechanisms to prevent the harmful aggregation of polyQ-expanded proteins, including degradation through the ubiquitin-proteasome system and disaggregation by chaperones⁴⁻¹⁰.

At least nine human neurodegenerative diseases are associated with polyQ-containing proteins. Among them, Huntington's disease is caused by mutations in the exon 1 of the huntingtin (HTT) gene that expands the polyQ stretch of the protein¹². The wild-type HTT protein contains 6-35 polyQ repeats^{13,14} and does not aggregate even under stress conditions or during aging9. In individuals affected by Huntington's disease, an unstable expanded polyQ stretch (>Q35) causes aggregation and proteotoxicity. The pathogenic fragment of polyQ-expanded exon 1 of mutant HTT in different model organisms and human cells is sufficient to recapitulate key aspects of Huntington's disease, including pathological protein aggregation and cell death¹⁵⁻¹⁷. Another protein associated with a human disease is ATXN3, which can contain up to 52 polyQ repeats without forming aggregates even under challenging conditions9. However, a mutant polyQ extension beyond 52 repeats triggers ATXN3 aggregation, causing Machado-Joseph disease^{18,19}.

While plants express hundreds of proteins containing polyQ regions²⁰, no pathologies arising from these proteins have been reported to date. In contrast to human HTT and ATXN3, which have relatively long polyQ repeats in their wild-type forms, the polyQ stretch in the Arabidopsis thaliana proteome does not exceed 24 repeats²⁰. Interestingly, specific polyQ-proteins act as sensors that integrate internal and external cues, enabling Arabidopsis to adapt to its ever-changing environment²¹⁻²³. One example is the transcription factor EARLY FLOWERING 3 (ELF3), which contains a Q7 stretch that allows the plant to respond to high temperatures through its aggregation. At 22° C., ELF3 remains soluble and binds to genes that repress flowering. At temperatures higher than 27° C., ELF3 forms aggregates that relieve transcriptional repression and promote flowering^{21,24} Thus, ELF3 can form aggregates in Arabidopsis under stress conditions even with its relatively short Q7 motif²¹.

Since the longest polyQ expansion in Arabidopsis proteins is 24 repeats²⁰, we expressed the exon 1 of human HTT containing Q28 and Q69 to examine whether plants can cope with polyQ-expanded proteins. Under normal conditions, neither Q28 nor Q69 leads to the formation of aggregates or deleterious effects in Arabidopsis. However, similar to Arabidopsis ELF3 (Q7)²¹, both Q28 and Q69 accumulate into aggregates upon heat stress. Under non-stress conditions, Arabidopsis efficiently prevents aggregation of polyQ-expanded proteins through their import and degradation into the chloroplast. Conversely, disruption of chloroplast proteostasis either pharmacologically or genetically triggers the cytosolic aggregation of Q69 as well as endogenous polyQ-proteins. We found that both Q28 and Q69 interact with various chloroplast proteins, such as the stromal processing peptidase (SPP). Notably, ectopic expression of SPP reduces the aggregation of polyQ-expanded proteins in human cells and nematode models. These findings support the development of new strategies for therapeutic, plant-based proteins that could target human polyQ diseases.

Huntington's disease remains incurable; however, some drugs based on antisense oligonucleotides have been designed to lower the levels of the Huntingtin mutant protein, but development has stalled. Following the drugs' disappointing performance, Roche and Wave Life Sciences stopped clinical trials of gene-targeting therapies for Huntington's disease (HD). (Diana Kwon (2021)). Unfortunately, the drug suppresses the production of the healthy and the mutant form of Huntingtin, and a decrease in levels of the normal protein could have caused problems. Other possibilities are that the antisense oligonucleotides did not reach the right brain parts (Diana Kwon (2021)).

In contrast to gene silencing strategies that aim to reduced expression of expanded polyQ proteins, we proposed to use a protein with antiaggregation/disaggregating activity. We have previously identified the plant chloroplast stromal processing peptidase (SPP) as a protein capable of disaggregating mutant Huntingtin protein in human cells, the protein that causes Huntington's disease. Finally, there is a need for drugs for the treatment of Huntington's disease (HD) which do not affect the expression of healthy proteins and which do not cause side effects due to manipulation of the expression of the healthy (normal, wildtype) proteins (see Table 1). There is further a need for polypeptide based drugs for the treatment of Huntington's disease (HD) and other polyQ stretch associated diseases which reduces aggregation of proteins comprising an extended polyQ stretch and that subsequently reduces neurotoxicity.

### BRIEF SUMMARY OF THE INVENTION

It is an object of the present invention to provide a new biological agent or biopharmaceutical for the treatment of neurogenerative diseases. It is further an object of the present invention to provide a polypeptide with an antiaggregating activity and/or disaggregating activity toward pathological proteins that cause neurogenerative diseases such as Huntington's disease and that have neurotoxic effect on the patient. Considering the disadvantages and side effects of the prior art, it is an object to provide a polypeptide based drug that does not affect expression of healthy counterparts of the pathologic proteins (Table 1) and that reduces aggregation of proteins comprising an extended polyQ stretch and that subsequently reduces neurotoxicity. It is the object to provide a polypeptide that is a plant derived protein, derived from human or that is a chimeric protein enabling the treatment of neurogenerative diseases. To achieve such therapeutic proteins, tools and a method for genetic modification of potential proteins according to the present invention are provided as well as a method for the genetic modification of isolated cells. Therefore, it is an object of the present invention to provide a method for the treatment of neurogenerative diseases such as Huntington's disease, wherein a protein/therapeutic protein that reduces aggregation of pathological proteins comprising an extended polyQ stretch is provided and that subsequently reduces neurotoxicity. Another object is the provision of a nucleic acid construct for the expression of a suitable protein that exhibits a antiaggregating activity and/or disaggregating activity towards pathological proteins (target proteins).

The present application provides a technical solution for the problem as defined in the claims, feasibly shown in the provided experiments and figures, and further explained in the embodiments.

The first aspect of the present invention is an in particular isolated, protein exhibiting an antiaggregating activity and/or disaggregating activity toward a target protein comprising an extended polyQ stretch and wherein the protein exhibiting said antiaggregating activity and/or disaggregating activity comprises a Zn²⁺-binding region. The protein of the present invention is an isolated protein. Preferably, the protein of the present invention or any embodiment is a therapeutic protein, in particular for use in the treatment or prevent of neurogenerative disease as defined herein. It has been isolated from a eukaryotic organism, a mammalian cells, a human cell and/or from a plant. The protein exhibits the antiaggregating activity and/or disaggregating activity in eukaryotic cells, preferably in mammalian cell, more preferably in human cells. The antiaggregating activity and/or disaggregating activity comprises unfolding/disaggregation of the extended polyQ stretch. It may also comprise cleavage or break down of the extended polyQ stretch into fragments. The protein preferably has a chaperon function or it is an enzyme.

In an embodiment of the protein according to the present invention, its amino acid (aa) sequence comprises a conserved motif HxxEHx₇₅₋₈₀E, in particular in the Zn²⁺-binding region, wherein x is any amino acid, optionally wherein the enzyme comprises at least one conservative amino acid substitution or a deletion in at least one or more positions x compared to the corresponding amino acid in the naturally (synonym wildtype) occurring enzyme from which the motif is derived. Preferably, the conserved motif is HxxEHx75E, HxxEHx₇₆E, HxxEHx₇₇E, HxxEHx₇₈E, HxxEHx₇₉E or HxxEHx₈₀E. The conserved motif HxxEHx₇₅₋₈₀E determines the Zn²⁺-binding region. The Zn²⁺-binding region is essential for the antiaggregating activity and/or disaggregating activity, optionally for the peptidase activity.

In another embodiment of the protein according to the present invention, the Zn²⁺-binding region supports or enables an interaction with at least one carbonyl group of the target protein, preferably it supports or enables a nucleophilic attack on the at least one carbonyl group of the target protein.

Preferably, the protein of the present invention or any embodiment is a therapeutic protein, that supports or enables an interaction with at least one carbonyl group of pathologic protein in the use in the treatment or prevent of neurogenerative disease as defined herein.

In another embodiment of the present invention, a variant of the protein according to the present invention, comprising at least one mutation in at least one position of H or E at any non-x location in the conserved motif, exhibits a decreased antiaggregating activity and/or disaggregating activity or full loss of antiaggregating activity and/or disaggregating activity, preferably it is a decreased peptidase activity or full loss of said activity (peptidase activity).

With regard to the Zn²⁺ biding region, the cleavage of the peptide bond of the substrate (target protein) occurs by means of a reaction similar to that of thermolysin, where a water molecule complexed to the Zn²⁺ ion is polarized by nearby glutamate, thereby allowing it to carry out a nucleophilic attack on the carbonyl of the peptide bond.

In another embodiment of the protein according to the present invention, it is a variant that comprises at least one or more mutations, deletions and/or substitutions in at least one or more positions of any x location of the conserved motif. Preferably said variant exhibits essentially the same protein activity, preferably the protein activity is the antiaggregating activity and/or disaggregating activity according to the present invention, optionally it is a peptidase activity.

In another embodiment, the protein is an enzyme comprising a Zn²⁺ binding region and determined by the conserved motif HxxEHx₇₅₋₈₀E, wherein x is any amino acid.

In a preferred embodiment of the protein of the present invention, the protein lacks a functional N-terminal signal peptide for translocation through a lipid bilayer, preferably through a lipid bilayer for import into mitochondria, ER or chloroplast. Alternatively, the protein lacks such a N-terminal signal peptide at all. The naturally occurring protein (without any genetic modification) comprises at least one functional signal peptide for translocation through a lipid bilayer into mitochondria, ER or chloroplast. In MPP, e.g. each subunit comprises such a signal peptide for import into mitochondria. According to the present invention, the lack of such an N-terminal signal peptide results in abrogation of translocation into mitochondria, ER or chloroplasts of the protein of the present invention. The nucleic acid sequence of beta MPP without the signal peptide is shown in Seq ID No. 5 and of alpha MPP in Seq ID No. 6. The corresponding amino acid sequences are shown in Seq ID No. 24 and 25. In any embodiment of the protein—as defined herein—said N-terminal signal peptide is deleted, knocked out or not expressed in order to ensure abrogation of import of said protein into. chloroplasts or mitochondria. Consequently, the respective nucleic acid construct and/or mRNA of the present invention does not encode said signal peptide, encodes a dysfunctional signal peptide, or comprises any other molecular modification ensuring abrogation of import of said protein into chloroplasts or mitochondria. Preferably, in the respective protein of the present invention the nucleic acid sequence encoding said signal peptide is removed and a new start codon is added (Seq ID No. 5, Seq ID No. 6). Preferably, the protein of the present invention or any embodiment is a therapeutic protein, that does not comprise a functional or no signal peptide for import of said protein into chloroplasts or mitochondria. Thereby, in the use in the treatment or prevention of neurogenerative diseases as defined herein, the therapeutic protein exhibits an antiaggregating activity and/or disaggregating activity toward a target protein comprising an extended polyQ stretch. It preferably supports or enables an interaction with at least one carbonyl group of a pathologic protein (target protein) that is present in the diseased cells.

In another embodiment of the present invention, the protein further comprises a glycine-rich loop of at least 5, 6, 7, or 8 glycine residues, optionally wherein the glycine residues are not contiguous. Preferably, the protein comprises the glycine-rich loop an the Zn²⁺ binding region as defined herein, preferably the conserved motif HxxEHx₇₅₋₈₀E and preferably does not contain a functional N-terminal signal peptide or no N-terminal signal peptide at all, preferably for translocation through a lipid bilayer for import into mitochondria, ER or chloroplast.

Within the meaning of the present invention, the glycine-rich loop, in particular in MPP or any derivative thereof, controls access to the catalytic site of the protein, preferably of the wildtype enzyme. In another embodiment of the protein of the present invention, the glycine-rich loop has the amino acid sequence GGGGSFSAGGPGKGMFS (Seq ID No. 28) or any amino acid sequence, wherein the amino acids other than G are arbitrarily exchangeable. The protein of the present invention may comprise an amino acid sequence, wherein the amino acids other than G are arbitrarily exchanged being at least 45% homologous to Seq ID No. 28, at least 50%, at least 55%, at least 60%, at least, 65%, at least 70% or at least 75% homologous to Seq ID No. 28. Preferably, the glycine rich loop has no changes in any G as depicted in Seq ID No. 28. The protein preferably is a mitochondrial-processing peptidase (MPP), the alpha subunit, a fragment of the alpha subunit comprising the glycine rich group, or any derivative of the MPP, wherein derivatives comprise fusion proteins and chimeric proteins as defined herein.

In another embodiment of the protein of the present invention, its target protein comprising an extended polyQ stretch is a variant of its respective wildtype protein comprising a wildtype polyQ stretch, preferably the target protein is selected from the group consisting of HTT, Ataxin-1, Ataxin-2, Ataxin-3, Ataxin-7, CACNA1A, TBP, Atrophin-1 and androgen receptor.

In another embodiment of the protein of the present invention, its target protein comprising the extended polyQ stretch is intrinsically disordered and/or improperly folded (in particular misfolded) and preferably prone to aggregation, more preferably the target protein has at least one or more prion-like domains. In another embodiment the extended polyQ stretch comprises an increased amount of Glutamine (Q) repeats compared to the respective wildtype protein comprising a wildtype polyQ stretch, preferably the extended polyQ stretch of the target protein comprises at least one glutamine (Q) more compared to the normal range of Q repeats of the respective wildtype protein, preferably at least Q₂ to Qₙ (QQ-Qn) more compared to the normal (wildtype) range of Q repeats, wherein n is an integer in a range of 1-200. Whether the extended polyQ stretch causes aggregation of the protein, causes a disease or disorder depends on the specific protein. It also depends on the age of the patient whether the pathologic condition is caused or triggered. In an embodiment of the protein of the present invention, the target protein comprising the extended polyQ stretch is pathogenic for an organism, preferably for humans. The pathologic condition may also be caused by the presence (increasing concentration) of mRNA molecules encoding for the target protein comprising the extended polyQ stretch. An extended polyQn comprises at least 18 Q repeats (n=18), at least 19 Q repeats or more, at least 20 Q repeats, at least 21 Q repeats (preferably for CACNA1A), at least 22 Q repeats, at least 23 Q repeats, at least 24 Q repeats, at least 25 Q repeats, at least 26 Q repeats, at least 27 Q repeats, at least 28 Q repeats, at least 29 Q repeats, at least 30 Q repeats, at least 31 Q repeats, at least 32 Q repeats, at least 33 Q repeats, at least 34 Q repeats (preferably for Ataxin-2), at least 35 Q repeats, at least 36 Q repeats (preferably for HTT), at least 37 Q repeats, at least 38 Q repeats (preferably for Ataxin-7, or Androgen receptor), at least 39 Q repeats, at least 40 Q repeats, at least 41 Q repeats (preferably for Ataxin-1), at least 42 Q repeats, at least 43 Q repeats, at least 44 Q repeats, at least 45 Q repeats (preferably for TBP), at least 46 Q repeats, at least 47 Q repeats, at least 48 Q repeats, at least 49 Q repeats (preferably for Atrophin-1) or more up to 200 Q repeats.

The skilled person knows different proteins comprising naturally occurring polyQ stretches and the respective variants comprising an extended polyQ stretch. Some examples are shown in Table 1. For example, an aggregation-prone target protein comprises a polyQ stretch of greater than 18 glutamine residues (preferably for an Ataxin protein), optionally greater than 35 glutamine residues (preferably for HTT), further optionally greater than 52 glutamine residues. Greater than 39 for Ataxin 1, greater than 32 for Ataxin 2, greater than 18 for Ataxin 7 and for CACNA1A, great than 43 for TBP, greater than 40 for Ataxin 3, greater than 38 for Atrophin.

The target protein may be present as a single target protein comprising an extended polyQ stretch with at least one or more prion-like domains. It may also be present in an accumulation of at least one or more target proteins. The accumulation may be an aggregate comprising at least one or more target proteins—as described herein—each comprising an extended polyQ stretch and optionally each comprising one or more prion-like domains.

In a further embodiment of the protein of the present invention, the, in particular isolated, protein comprises one or more subunits, at least one or more fragments of any subunit of an, in particular isolated, protein, at least one or more fragments of an, in particular isolated, protein, or it is a derivative of any combination of the aforementioned components. In another embodiment the protein is a plant derived stromal processing peptidase (SPP), a genetically modified SPP, a human derived and SPP-like protein, a human derived and genetically modified SPP-like protein, a fusion protein or a hybrid protein. Preferably, any derivative and SPP-like protein of the present invention exhibit an antiaggregating activity and/or disaggregating activity toward a target protein comprising an extended polyQ stretch. Therefore, those inventive derivatives and SPP-like proteins exhibit essentially the same technical effect of disaggregation of target proteins comprising a polyQ stretch and which are prone to aggregation. The inventive derivatives and SPP-like proteins optionally exhibit the same technical effect of prevention of aggregation of nascent polypeptides of the target protein while expressed from the mRNA. Preferably, the SPP-like protein comprises at least the conserved motif HxxEHx₇₅₋₈₀E as defined herein and do not contain a functional N-terminal signal peptide or no N-terminal signal peptide at all. Optionally, the SPP-like protein is an SPP-like enzyme exhibiting a peptidase activity.

In a preferred embodiment of the present invention, the protein is a chimeric protein composed of at least one component derived from a human and at least a second component from another organism, preferably from a plant. The chimeric protein exhibits an antiaggregating activity and/or disaggregating activity toward a target protein comprising an extended polyQ stretch, it preferably comprises at least the conserved motif HxxEHx₇₅₋₈₀E as defined herein and does not contain a functional N-terminal signal peptide or no N-terminal signal peptide at all, preferably for translocation through a lipid bilayer for import into mitochondria, ER or chloroplast. Optionally, it comprises a glycine rich loop of the alpha MPP subunit. A chimeric (optionally fusion) protein may comprise, without limiting the invention:
an alpha MPP subunit with the naturally occurring glycine rich loop or with a genetically modified glycine rich loop and a fragment of the SPP comprising the motif HxxEHx₇₅₋₈₀E without any N-terminal signal peptide for translocation through a lipid bilayer for import into mitochondria, ER or chloroplasts,
an alpha MPP subunit with the naturally occurring glycine rich loop or with a genetically modified glycine rich loop and a fragment of the SPP comprising the motif HxxEHx₇₅₋₈₀E without a functional N-terminal signal peptide for translocation through a lipid bilayer for import into mitochondria, ER or chloroplasts,
a fragment of Nardilysin and a fragment of the SPP comprising the motif HxxEHx₇₅₋₈₀E, without any N-terminal signal peptide for translocation through a lipid bilayer for import into mitochondria, ER or chloroplasts,
a fragment of Nardilysin and a fragment of the SPP comprising the motif HxxEHx₇₅₋₈₀E, without a functional N-terminal signal peptide for translocation through a lipid bilayer for import into mitochondria, ER or chloroplasts,
a fragment of Insulin-Degrading Enzyme and a fragment of the SPP comprising the motif HxxEHx₇₅₋₈₀E, without any N-terminal signal peptide for translocation through a lipid bilayer for import into mitochondria, ER or chloroplasts, or
a fragment of Insulin-Degrading Enzyme and a fragment of the SPP comprising the motif HxxEHx₇₅₋₈₀E, without a functional N-terminal signal peptide for translocation through a lipid bilayer for import into mitochondria, ER or chloroplasts.

Preferably, the chimeric protein of the present invention is a therapeutic protein, that does not comprise a functional or no signal peptide for translocation through a lipid bilayer for import into mitochondria, ER or chloroplast. Thereby in the use in the treatment or prevention of neurogenerative disease as defined herein, the therapeutic protein exhibits an antiaggregating activity and/or disaggregating activity toward a target protein comprising an extended polyQ stretch. By means of the use of the present invention the protein reduces aggregation of proteins comprising an extended polyQ stretch and subsequently reduces neurotoxicity. It preferably supports or enables an interaction with at least one carbonyl group of pathologic protein (target protein) of the diseased cells.

The nucleic acid construct (and respective mRNA) of the present invention, encodes for a chimeric protein according to the present invention, at least encoding for SPP without the signal peptide (Seq ID No. 4) or a fragment thereof in combination with a sequence encoding for MPP alpha without the signal peptide (Seq ID No. 6) or for MPP beta without the signal peptide (Seq ID No. 5) or a fragment of MPP alpha or of MPP beta. In another embodiment of the combination of Seq ID No. 4, 5 and/or 6, the sequences encoding for GFP has been removed from Seq ID No. 4. Another aspect is a nucleic acid construct encoding the combination of SPP without the signal peptide or a fragment thereof with a sequence encoding for MPP alpha without the signal peptide or for MPP beta without the signal peptide or a fragment of MPP alpha or of for MPP beta, having any other sequence compared to Seq ID No. 4, 5 and/or 6 and encoding for a protein exhibiting an antiaggregating activity and/or disaggregating activity.

In a preferred embodiment, the protein of the present invention is a SPP-like protein exhibiting a antiaggregating activity and/or disaggregating activity toward a target protein comprising an extended polyQ stretch, preferably against HTT, Ataxin 1, Ataxin 2, Ataxin 7, CACNA1A, TBP, Ataxin 3 and/or Atrophin.

In a preferred embodiment of the present invention, the protein is a plant derived stromal processing peptidase (SPP) from an Arabidopsis species, preferably from Arabidopsis thaliana, a mitochondrial-processing peptidase (MPP) or any derivative thereof, Nardilysin or any derivative thereof, an Insulin-Degrading Enzyme (IDE) or any derivative thereof, or any combination of the aforementioned. Preferably, it is an isolated mitochondrial-processing peptidase (MPP) or any derivative thereof comprising the conserved motifs of the § (beta) subunit and optionally of the a subunit of MPP, preferably carrying the glycine rich loop of Seq ID No. 28. A further aspect is a fusion polypeptide (as defined above) comprising at least one element or motif respectively from the α (alpha) and/or beta MPP subunit and having the antiaggregating activity and/or disaggregating activity of the corresponding wildtype MPP dimer.

Another aspect of the present invention is a SPP-like protein wherein the protein comprises a non-functional Zn²⁺-binding region, that comprises at least one mutation in the conserved motif and having antiaggregating activity and/or disaggregating activity toward extended polyQ stretch containing proteins.

Preferably, the protein within the meaning of the present invention is a mutant protein comprising a Zn²⁺-biding region and having TM-Score of at least 0.5, at least 0.55, at least 0.575, of at least 0.6, of at least 0.605, of at least 0.61, of at least 0.615, of at least 0.62, of at least 0.625, of at least 0.63, of at least 0.635, of at least 0.64, of at least 0.645, of at least 0.65, of at least 0.655, of at least 0.66 or more (see e.g. FIGS. 12B and 13B) compared to SPP from Arabidopsis thaliana (Seq ID No. 26), and which exhibits a antiaggregating activity and/or disaggregating activity toward the target protein comprising an extended polyQ stretch according to the present invention, more preferably in a mammalian cell, human cells or in cell-free systems. TM-score, also known as template modeling score, is a bioinformatics metric used to assess the similarity between two protein structures.

Another aspect of the present invention is the protein as defined herein, which is expressed from a recombinant polynucleotide (synonym nucleic acid construct), optionally a codon-optimized polynucleotide (synonym nucleic acid construct), further optionally under the control of a heterologous promoter.

Therefore, another aspect of the present invention is a nucleic acid construct encoding for the protein that exhibits an antiaggregating activity and/or disaggregating activity toward a target protein comprising an extended polyQ stretch as defined herein, wherein the nucleic acid construct comprises a sequence encoding a Zn²⁺ binding region, preferably comprising a sequence encoding an amino acid sequence comprising the conserved motif HxxEHx₇₅₋₈₀E. Preferably, the nucleic acid construct encodes for the conserved motif HxxEHx₇₅E, HxxEHx₇₆E, HxxEHx₇₇E, HxxEHx₇₈E, HxxEHx₇₉E or HxxEHx₈₀E. The nucleic acid construct preferably does not encode a functional N-terminal signal peptide or it does not encode an N-terminal signal peptide at all for translocation through a lipid bilayer for import into mitochondria, ER or chloroplasts. According to the present invention, the lack of a sequence encoding a functional N-terminal signal peptide results in abrogation of import into the mitochondria or chloroplasts of the expressed protein of the present invention. The sequence naturally encoding for the functional signal peptide is at least partially deleted, is knocked out or not encoded at all by the nucleic acid construct.

Where the functional N-terminal signal peptide naturally guides the expressed protein through a lipid layer of chloroplasts, mitochondria or ER and allows translocation through a lipid bilayer, for import into the organelle, a dysfunctional signal peptide abrogates said import of the protein of the present invention. In a preferred embodiment the nucleic acid construct does not comprise any sequence encoding for such a signal peptide. Consequently, the respective nucleic acid construct and/or mRNA of the present invention does not encode for such a signal peptide, encodes for a dysfunctional signal peptide or comprises any other molecular modification ensuring abrogation of import of the protein into the mitochondria or chloroplasts.

In another embodiment of nucleic acid construct of the present invention, the nucleotide sequence encoding the protein of the present invention is optionally codon-optimized and wherein the nucleic acid construct optionally comprises a sequence encoding a heterologous promoter. The heterologous promoter controls the expression, in vivo, ex vivo or in vitro, of the protein according to the invention.

A further aspect of the present invention is a mRNA sequence, in particular a mRNA molecule, encoding a protein that exhibits an antiaggregating activity and/or disaggregating activity toward a target protein comprising an extended polyQ stretch of any one of the proceeding claims, wherein the mRNA encodes a Zn2+-binding region, preferably the mRNA comprises a sequence encoding an amino acid sequence comprising the conserved motif HxxEHx₇₅₋₈₀E. In consequence the sequence of mRNA molecule of the present invention is determined by the sequences of the inventive nucleic acid constructs. Thus, any embodiment of the nucleic acid constructs applies accordingly for the mRNA molecule of the present invention.

Another aspect of the present invention is the use of the protein exhibiting an antiaggregating activity and/or disaggregating activity toward a target protein comprising an extended polyQ of the present invention in a method of inhibiting or preventing aggregation of an aggregation-prone polyQ protein or a fragment thereof, which method comprises allowing a protein comprising a Zn²⁺-binding region exhibiting antiaggregating activity and/or disaggregating activity to come into contact with the aggregation-prone polyQ protein or fragment thereof.

The aforementioned method of disaggregating aggregates of an aggregation-prone polyQ protein or a fragment thereof, may include allowing a protein comprising a Zn²⁺-binding region and exhibiting a disaggregating activity to come into contact with the aggregates. In said method the aggregation-prone polyQ protein or fragment thereof preferably comprise a stretch of greater than 18 glutamine residues, optionally greater than 35 glutamine residues, further optionally greater than 52 glutamine residues. More preferably, the stretch of glutamine residues is contiguous.

In another embodiment of the method of disaggregating aggregates of an aggregation-prone polyQ protein, the protein may include a plant derived stromal processing peptidase (SPP) from an Arabidopsis species, preferably from Arabidopsis thaliana, a mitochondrial-processing peptidase (MPP), Nardilysin, an Insulin-Degrading Enzyme (IDE) or any combination of the aforementioned proteins or of any fragments of the aforementioned proteins.

In another aspect, the method of disaggregating aggregates of an aggregation-prone polyQ protein or any embodiment may be an in vitro method, an ex vivo method, or an in vivo method.

In another embodiment, the method of disaggregating aggregates of an aggregation-prone polyQ protein may include expressing the protein in a eukaryotic cell, preferably in a mammalian cell, even more preferably in a human cell, under conditions in which the aggregation-prone polyQ protein or fragment thereof is disaggregated, cleaved, or both disaggregated and cleaved.

In another embodiment, the method of disaggregating aggregates of an aggregation-prone polyQ protein may include such a method wherein the aggregation-prone polyQ protein or a fragment thereof is: (i) a Huntingtin (HTT) protein or a variant thereof comprising an expanded polyQ sequence compared to wild type HTT protein, or (ii) Ataxin-3 (ATXN3) or a variant thereof comprising an expanded polyQ sequence compared to wild type ATXN3.

Another aspect of the present invention is the nucleic acid construct or mRNA sequence of the present invention for use in a method for prevention or treatment of a neurodegenerative disease that is caused by aggregates comprising at least one target protein comprising extended polyQ stretches and/or by mRNA molecules encoding for target proteins, wherein the expressed protein (i) exhibits an antiaggregating activity and/or disaggregating activity toward said aggregates and/or (ii) prevents aggregation of the nascent polypeptide of the target protein, preferably of the expressed (released from ribosome) or expressing protein (while expression in ongoing). Preferably, the use in a method for prevention or treatment of a neurodegenerative disease comprises the steps of
providing the protein exhibiting a antiaggregating activity and/or disaggregating activity according to the present invention in a formulation suitable for administration; and
administering a suitable amount of the protein exhibiting an antiaggregating activity and/or disaggregating activity according to the present invention to a subject suffering from a neurodegenerative disease, preferably Huntington's disease, in particular caused by expanded polyQ variants of HTT as described herein or P42858, and Machado-Joseph disease, in particular caused by the expanded polyQ variants of Ataxin-3 (ATXN3)—P54252.

In a preferred embodiment of the use of the present invention the neurodegenerative disease comprises Huntington's disease and Machado-Joseph disease, preferably caused by the target protein comprising an extended polyQ stretch (e.g. expanded polyQ variants of HTT as described herein or of expanded polyQ variants of P42858, Ataxin-3 (ATXN3)—P54252). Some examples of pathologic target proteins are shown in Table 1.

Another aspect of the present invention is a method of ex vivo or in vitro genetic modification of isolated cells, preferably human cells, with the nucleic acid construct and/or mRNA of any one of the of the present invention. The method preferably comprises delivering the nucleic acid construct and/or mRNA of the present invention into an isolated cell, e.g., via transfection, optionally with a plasmid or viral vector, preferably an adenoviral vector.

Another aspect of the present invention is an isolated eukaryotic cell, preferably a human cell, which expresses an exogenous polypeptide, preferably that expresses a protein exhibiting an antiaggregating activity and/or disaggregating activity toward a target protein comprising an extended polyQ stretch according to the present invention. In another embodiment the isolated cell expresses an exogenous polypeptide selected from a stromal processing peptidase (SPP) having 50% or greater sequence identity to SEQ ID NO: 26, having 60% or greater sequence identity to SEQ ID NO: 26, having 70% or greater sequence identity to SEQ ID NO: 26, a mitochondrial-processing peptidase (MPP) having 70% or greater sequence identity to SEQ ID NO: 26, Nardilysin having 50%, 60% or 70% or greater sequence identity to SEQ ID NO: 26, an Insulin-Degrading Enzyme (IDE) having 50%, 60% or 70% or greater sequence identity to SEQ ID NO: 26, or a fragment or a derivative of any of these. In another embodiment the isolated cell expresses an exogenous polypeptide selected from a stromal processing peptidase (SPP) having TM-Score of at least 0.5, at least 0.55, at least 0.575, of at least 0.6, of at least 0.62, of at least 0.61, of at least 0.615, of at least 0.62, of at least 0.625, of at least 0.63, of at least 0.635, of at least 0.64, of at least 0.645, of at least 0.65, of at least 0.655, of at least 0.66 or more (see e.g. FIGS. 12B and 13B) compared to SPP from Arabidopsis thaliana (Seq ID No. 26) and which exhibits an antiaggregating activity and/or disaggregating activity toward the target protein comprising an extended polyQ stretch according to the present invention, more preferably in a mammalian cell, human cells or in cell-free systems.

The isolated cell is preferably a mammalian cell, preferably a human cell. The isolated eukaryotic, which is a cell of the central nervous systems, e.g., a neuron or glial cell. The isolated eukaryotic cell of the present invention, wherein the glial cell is an astrocyte, oligodentrocyte, ependymal cell, or microglial cell.

Another aspect of the present invention is a method of screening for a polypeptide, preferably an protein, capable of antiaggregating activity and/or disaggregating, preferably unfolding, dissolving or cleaving, an aggregate of at least one or more target proteins comprising respectively an extended polyQ stretch compared to the respective wildtype protein comprising a wildtype polyQ stretch, wherein the method comprises the steps
providing a predetermined polypeptide candidate (preferably any one of the defined herein)
contacting the polypeptide candidate with at least one or more aggregation-prone polypeptides containing an extended polyQ stretch of with at least one or more aggregates as defined herein,
analyzing whether the aggregates are disaggregated, preferably by microscopy (particle sizes), western blot analysis, analysis of molecular weight or any other method suitable to determine proteins, and
optionally a polypeptide candidate is identified positive for exhibiting an antiaggregating activity and/or disaggregating activity toward a target protein comprising an extended polyQ stretch.

Preferably, the analysis is performed with isolated cells, wherein the cells express the polypeptide candidate and the target protein. Preferably, whether any aggregates are disaggregated is observed intracellular. The method is performed in vitro or ex vivo with isolated human cells.

Another aspect of the present invention is a method of screening for a polypeptide, preferably a protein capable of preventing aggregation of an aggregation-prone target protein, which method comprises allowing a candidate substance to come into contact with the aggregation-prone polypeptide or fragment thereof; and determining whether aggregation of the aggregation-prone polypeptide is inhibited or prevented.

For analytics in any of the aforementioned screening methods, the filter trap method as used in the examples or fluorescent measurements are suitable to analyze whether aggregates are present or disaggregated according to the present invention. Further western blot analyses are suitable to identify the number and size of polypeptide. The advantage of microscopic analysis is that the size (particle size) and amount of different aggregates are detectable.

In an embodiment of the screening method the aggregation-prone polypeptide is a polyQ-stretch-containing protein and the candidate substance is a protein exhibiting the antiaggregating activity and/or disaggregating activity as defined herein. The aggregation-prone polypeptide containing an extended polyQ stretch or at least one or more aggregates preferably comprises at least one tag, optionally a fluorescent tag.

In an embodiment of the screening method the aggregates or the disaggregated target proteins as defined herein are determined based on the molecular weight and/or migration behavior in a gel. The determination is performed after the aggregation-prone polypeptide or fragments thereof have been incubated and come into contact with the polypeptide candidate (potential protein of the present invention exhibiting a disaggregation activity as defined herein). Preferably, the filter trap method as disclosed in Llamas et al 2023 is used.

The screening method of the present invention is performed in vitro or in vivo. The in vitro method comprises isolated cells or cell lines or a cell-free environment suitable for protein interaction. The in vivo method encompasses a living eukaryotic organism, preferably C. elegans worms and a recombinant mice model, wherein the desired target protein, preferably pathologic protein, is expressed. Preferably, the living eukaryotic organism, preferably C. elegans and mice, exhibits the pathological phenotype e.g. of Huntington's diseases or Machado-Joseph disease.

The screening method, wherein determining whether aggregation of the aggregation-prone polypeptide is inhibited or prevented comprises observing whether cleavage of the aggregation-prone polypeptide occurs and cleavage indicates that the candidate substance is capable of inhibiting or preventing aggregation of the aggregation-prone polypeptide. In the in vivo embodiment of the screening method the motility of the organism is observed (video, images) and/or by means of microscopy, preferably by fluorescence microscopy, the localization and/or distribution of the aggregation-prone polypeptide or of the disaggregated target protein is observed.

In the screening method of the present invention determining whether aggregation of the aggregation-prone polypeptide is inhibited or prevented comprises observing intracellular localization and/or distribution of the aggregation-prone polypeptide with or without cleavage of the aggregation-prone polypeptide, preferably by fluorescence microscopy.

Another aspect of the present invention is a method of inhibiting or preventing aggregation of an aggregation-prone polyQ target protein or a fragment thereof, which method comprises allowing an protein comprising a Zn²⁺-binding region exhibiting antiaggregating activity and/or disaggregating activity to come into contact with the aggregation-prone polyQ target protein or fragment thereof.

In an embodiment, such a method of antiaggregating and/or disaggregating aggregates of proteins comprising extended polyQ stretches includes allowing a protein comprising a Zn2+-binding region and exhibiting a antiaggregating activity and/or disaggregating activity to come into contact with the aggregates. The method may be an in vitro method, an ex vivo method, or an in vivo method.

The method above may also include such a method in which the aggregation-prone polyQ target protein or aggregate comprises a stretch of greater than 18 glutamine residues, optionally greater than 35 glutamine residues, further optionally greater than 52 glutamine residues. In such a method of the present invention, the stretch of glutamine residues may be contiguous. In such a method the protein may comprise a plant derived stromal processing peptidase (SPP) from an Arabidopsis species, preferably from Arabidopsis thaliana, a mitochondrial-processing peptidase (MPP), Nardilysin, an Insulin-Degrading Enzyme (IDE) or any combination of the aforementioned proteins or of any fragments of the aforementioned proteins.

In an embodiment, such a method of the present invention comprises expressing the protein in a eukaryotic cell, preferably in a mammalian cell, even more preferably in a human cell, under conditions in which the aggregation-prone polyQ protein or the aggregates are disaggregated, cleaved, or both disaggregated and cleaved. In the method the aggregate comprises or the aggregation-prone polyQ protein or a fragment thereof is: (i) a Huntingtin (HTT) protein or a variant thereof comprising an expanded polyQ sequence compared to wild type HTT protein, or (ii) Ataxin-3 (ATXN3) or a variant thereof comprising an expanded polyQ sequence compared to wild type ATXN3 that causes Machado-Joseph disease.

Another aspect of the present invention is a method of treating a polyQ-based disease comprising administering to a subject in need thereof an effective amount of a composition comprising a polypeptide configured to prevent or inhibit intracellular aggregation of at least one extended polyQ-stretch-containing protein.

In an embodiment of such a method, the polypeptide disaggregates the at least one polyQ-stretch-containing protein.

In an embodiment of such a method, the method comprises administering the composition to a subject having or at risk of having Huntington's disease or Machado-Joseph disease.

In an embodiment of such a method, the at least one polyQ-stretch-containing protein is Huntingtin (HTT) protein or a variant thereof comprising an expanded polyQ sequence compared to wild type HTT protein.

In an embodiment of such a method, the HTT protein or variant thereof comprises a stretch of greater than 35 glutamine residues, optionally wherein the stretch of glutamine residues is contiguous.

In an embodiment of such a method, the at least one polyQ-stretch-containing protein is ATXN3 or a variant thereof comprising an expanded polyQ sequence compared to wild type ATXN3.

In an embodiment of such a method, the ATXN3 or variant thereof comprises a stretch of greater than 52 glutamine residues, optionally wherein the stretch of glutamine residues is contiguous.

### Definitions

The protein of the present invention, is a polypeptide of a certain amino acid, that exhibits an antiaggregating activity and/or disaggregating activity toward a target protein comprising an extended polyQ stretch as defined herein. The antiaggregating activity and/or disaggregating activity toward a target protein comprising an extended polyQ stretch is the essential functional feature of an inventive protein.

The protein according to the present invention comprises a Zn²⁺-binding region. In other words, the amino acid sequence of the polypeptide forming the active protein, comprises a sequence that determines and realizes a Zn²⁺-binding region in the active conformation of the protein. Preferably, the Zn²⁺-binding region comprises the a conserved motif HxxEHx₇₅₋₈₀E, preferably the motif is HxxEHx₇₅E, HxxEHx₇₆E, HxxEHx₇₇E, HxxEHx₇₈E, HxxEHx₇₉E or HxxEHx₈₀E. The Zn²⁺-binding region is essential for the protein activity, preferably for the peptidase activity. Some proteins according to the present invention, preferably enzymes, e.g. MPP comprises a glycine rich loop which, in particular in humans, is relevant for substrate binding, presently for binding of the target protein or its respective wildtype protein. The specific glycine rich loop of MPP has the sequence shown in Seq ID No. 28.

The protein may be isolated from an organism, eukaryotic organism, plants, mammalian cells or human cells. It may be genetically modified and/or it may comprise fragments and subunits.

Fragments and subunits may also be combined to create new derivates of a herein so called SPP-like protein that exhibits an antiaggregating activity and/or disaggregating activity toward a target protein comprising an extended polyQ stretch as defined herein and which preferably comprises at least the a conserved motif HxxEHx₇₅₋₈₀E (see above). SPP-like proteins comprise fusion proteins, hybrid proteins and chimeric proteins composed of one or more fragments, one or more subunits, respectively from one or more different organisms, or comprise any combination of the aforementioned, provided that the achieved SPP-like protein exhibits an antiaggregating and/or disaggregating activity toward a target protein comprising an extended polyQ stretch as defined herein and preferably comprises at least the conserved motif HxxEHx₇₅₋₈₀E. The protein of the present invention exhibiting an antiaggregating activity and/or disaggregating activity toward a target protein comprising an extended polyQ stretch does not comprise any N-terminal signal peptides which are capable of importing or translocating said protein into the chloroplasts, mitochondria or microsomes.

“SPP-like enzyme” refers to an enzyme which achieves the same technical effect of disaggregation of aggregation-prone target proteins comprising an extended polyQ stretch as SPP does. SPP-like enzymes comprise at least the conserved HxxEHx₇₅₋₈₀E motif. In one embodiment, an SPP-like protein may be an SPP-like enzyme having peptidase activity. Although the structure of the SPP-like enzyme may differ from SPP, the SPP-enzyme retains peptidase activity.

Said “antiaggregating activity” comprises the ability of the protein of the present invention to chaperone and/or fold and/or unfold and/or otherwise assist in a conformation change of the target protein. The protein's antiaggregating activity thereby reduces or even completely inhibits the target protein from being aggregation-prone. Said “antiaggregating activity” may be realized by means of assistance of the inventive protein in the conformational folding or unfolding of the target protein (including in its nascent form and/or during ribosomal synthesis) as defined herein and/or by a peptidase activity of the inventive protein.

Said “disaggregating activity” comprises the ability of the protein of the present invention to break down and/or resolve and/or solubilize already-formed aggregates of the target protein as defined herein. Said “disaggregating activity” may also be realized by means of assistance of the inventive protein in the conformational folding or unfolding of target protein as defined herein and/or by a peptidase activity of the inventive protein.

Target protein (synonym substrate) within the meaning of the present invention is any polypeptide, soluble protein (not membrane bound) protein, carrying an extended polyQ stretch compared to its respective wildtype protein comprising a wildtype (normal) polyQ stretch. It is a naturally occurring polypeptide of eukaryotic cells, preferably of mammalian cells, preferably human cells. The target protein has a variation in the length of the polyQ stretch, with is called the extended polyQ stretch, compared to its wildtype phenotype. The extended polyQ stretch comprises an increased amount of Glutamine (Q) repeats compared to the respective wildtype protein comprising a wildtype polyQ stretch, preferably the extended polyQ stretch of the target protein comprises at least one glutamine (Q) more compared to the normal range of Q repeats of the respective wildtype protein. More preferably at least Q2 to Qn (QQ-Qn) more compared to the wildtype range of Q repeats, wherein n is an integer in a range of 1-200. Due to said variation in length of the polyQ stretch the target protein comprises an “extended polyQ stretch”. The “extended polyQ stretch” is individual for each wildtype protein (see Table 1). e.g. for Huntingtin (HTT) a range of 6-35 Qs is a normal range, whereas for CACNA1A a polyQ21-30 is pathological. ATXN3 can contain up to 52 polyQ repeats, before becoming prone to aggregation. In contrast, the polyQ stretches in endogenous Arabidopsis proteins do not exceed 24 Q repeats. Due to the respective and protein-specific “extended polyQ stretch” the target protein is prone to aggregation. The mRNA molecules encoding for the target proteins, the already expressed target proteins and/or the aggregates thereof cause a disorder or diseases and a pathological condition. In the disease state the target protein comprising the extended polyQ stretch is intrinsically disordered and/or improperly folded (misfolded) and prone to aggregation. The target protein may comprise one or more prion-like domains.

Finally, the variation of the target protein causes a disease and the disease is preferably a neurodegenerative disease. Preferably, the target protein is selected from the group comprising Huntingtin, Ataxin-1, Ataxin-2, Ataxin-3, Ataxin-7, CACNA1A, TBP, Atrophin-1 and androgen receptor (Table 1, see also Fan et al 2014).

TABLE 1
Polyglutamine associated diseases and
their causative genes and proteins

| PolyQ | | | Expanded CAG Repeats | |
|---|---|---|---|---|
| Diseases | Locus | Protein | Normal | Pathological |
| SCA1 | 6p23 | Ataxin-1 | 6-39 | 41-83 |
| SCA2 | 12q24 | Ataxin-2 | 14-32 | 34-77 |
| SCA6 | 19p13 | CACNA1A | 4-18 | 21-30 |
| SCA7 | 3p21-p12 | Ataxin-7 | 7-18 | 38-200 |
| SCA17 | 6q27 | TBP | 25-43 | 45-63 |
| MJD/SCA3 | 14q24-q31 | Ataxin-3 | 12-40 | 62-86 |
| HD | 4p16.3 | Huntington | 6-35 | 36-121 |
| DRPLA | 12p13 | Atrophin-1 | 3-38 | 49-88 |
| SBMA | Xq11-q12 | Androgen receptor | 6-36 | 38-62 |

The skilled person knows very well in which databases amino acid sequences and derivates thereof are available, e.g. from uniprot Huntingtin (HTT)—P42858, Ataxin-3 (ATXN3)—P54252 (Machado-Joseph disease), Ataxin-2 (ATXN2)—Q99700, Ataxin-7 (ATX7)—015265 and CACNA1A—000555 are well known.

“polyQ” means a polypeptide sequence of a certain length defined by the number of glutamine (Q) repeats (Q1 to Qn, wherein n is an integer, preferably 1 to 200). For example, polyQ49 or Q49 refers to a polypeptide sequence consisting of 49 glutamine residues in a row. A polyQ stretch is part of the target protein.

A “fusion protein” as contemplated herein refers to a protein comprising one or more heterologous protein domains or one or more domains in addition to the conserved HxxEHx₇₅₋₈₀E motif. The fusion protein may comprise any additional protein sequence and optionally a linker sequence between any two domains.

A “hybrid” as contemplated herein refers to the combination of at least two different components derived from two different proteins, (IDE, MPP, SPP etc.) whereas the proteins are derived from the same organism

A “chimeric enzyme” as contemplated herein refers to a protein comprising any combination of the aforementioned enzymes or any combination of any fragments or subunits of the aforementioned enzymes, wherein components derived from two different organisms and at least one component is derived from a human cell, preferably it is a subunit or fragment of MPP.

### BRIEF DESCRIPTION OF THE DRAWINGS

FIGS. 1A-1I. Plants constitutively expressing polyQ-expanded proteins do not display aggregates or deleterious effects under normal conditions. FIG. 1A: Schematic representation of the constitutive constructs Q28 and Q69. PRD: Proline-rich domain. FIG. 1B: Phenotype of mature and senescent wild-type Col-0, Q28, and Q69 plants. Scale bar: 5 cm. Representative of 3 independent experiments.

FIG. 1C: Lifespan analysis of the fourth true leaf of Arabidopsis comparing Q28 and Q69 to control Col-0 plants. Scale bar: 1 cm. Representative of 3 independent experiments. FIG. 1D: Representative images of 46-day old plants (scale bar: 5 cm). The box plot represents the 25th-75th percentiles of the flowering time under short-day conditions at 22° C. (n=27 biological replicates), the line depicts the median and the whiskers are plotted following the Tukey method. FIG. 1E: Confocal images of Citrine-Q28 and Citrine-Q69 (citrine is a fluorescent protein derived from GFP) in epidermal pavement cells from cotyledons. 7-day-old seedlings grown at 22° C. were transferred in dark to incubators at 45° C. (heat stress (HS)) or 22° C. (control) for 90 minutes. Scale bar: 10 μm. Representative of three independent experiments. 1F: Quantification of the number of Q28 and Q69 aggregates per epidermal cell in 7-day-old cotyledons from the experiments presented in FIG. 1E (mean±s.e.m., Q28 n=6 cells from three independent experiments; Q28 HS n=9; Q69 n=6 Q69 HS n=7). FIG. 1G: Q28 and Q69 distribution in epidermal pavement cells from leaves of 22-day-old plants. The 4th leaf of Q28 or Q69 plants was dissected and incubated in dark under heat stress or control conditions for 90 minutes. Scale bar: 25 μm. Representative of three independent experiments. FIG. 1H: Quantification of Q28 and Q69 aggregates per epidermal cell in 22-day-old leaves from FIG. 1G (mean±s.e.m., n=6 cells per condition from three independent experiments;). FIG. 1I: Filter trap and SDS-PAGE analysis with anti-GFP antibody (capable of recognizing citrine tag) of the seedlings used for microscopy analysis in FIG. 1E. Rubisco large subunit (RbcL) is the loading control. Representative of two independent experiments. Statistical comparisons were made by two-tailed Student's t-test for unpaired samples.

FIGS. 2A-2E. Q28 and Q69 proteins interact with cytosolic and chloroplast proteostasis components in Arabidopsis. FIGS. 2A-2B: Co-immunoprecipitation (co-IP) experiments with anti-GFP antibody against Citrine-HTTexon1-Q28 (FIG. 2A) and Citrine- HTTexon1-Q69 (FIG. 2B) in transgenic 7-day-old Arabidopsis seedlings followed by quantitative label-free proteomics. For each biological replicate, the same amount of protein was incubated with either anti-GFP or negative control anti-IgG antibody. To identify significant interactors of Q28 and Q69, we compared protein abundance in GFP pulldowns with control IgG pulldowns. Volcano plots represent the −log 10(P-value) of a two-tailed t-test plotted against the log 2 ratio of protein label-free quantification (LFQ) values from GFP pulldown compared with control IgG pulldown (Student t-test, n=3 biological replicates). Gray and colored circles indicate significance after correction for multiple testing (False Discovery Rate (FDR)<0.05 was considered significant). Yellow circles: proteins involved in protein folding, red: proteins involved in chloroplast proteolytic degradation, orange: components of the chloroplast import machinery, blue: proteins involved in the ubiquitin-proteasome system (UPS), green: Q28 or Q69 proteins. FIG. 2C: Scheme indicating the subcellular localization of selected common interactors of Q69 and Q28. FIG. 2D: Co-IP with GFP and control IgG antibodies in Q69 seedlings followed by western blot against the chloroplast protease subunits ClpP4 and FtsH2/8. Representative of three independent experiments. FIG. 2E: Q28 and Q69 distribution in mesophyll cells of 7-day-old cotyledons. Images show Citrine fluorescence (green) and chloroplast autofluorescence (red). Scale bar: 5 μm. Representative of four independent experiments.

FIGS. 3A-3K. Reduced chloroplast proteostasis leads to Q69 aggregation. FIG. 3A: Confocal microscopy of isolated chloroplasts incubated with 10 μM recombinant polyQ69-HTTexon1 fused to Citrine (Q69-Citrine) or control HTTexon1-Citrine lacking the polyQ stretch (AQ-Citrine) at 25° C. under light for 30 min. TL: transmitted light. FIG. 3B: Higher magnification of isolated chloroplasts. In FIGS. 3A-3B, Scale bar: 5 μm. Images are representative of 4 independent experiments. FIG. 3C: Western blot with anti-GFP antibody of isolated chloroplasts incubated with 10 μM Q69-Citrine or AQ-Citrine for the indicated times. CBB: Coomassie Brilliant Blue is the loading control. PP: 0.1 μM of purified protein Q69-Citrine or AQ-Citrine was loaded for reference. Representative of 4 independent experiments. FIG. 3D: Western blot with anti-GFP of 7-day-old Q69 plants treated with mock or 800 μM lincomycin (LIN). RbcL is the loading control. Representative of 4 independent experiments. The graph shows relative Q69 protein levels to timepoint 0 (mean±s.e.m of 4 independent experiments, except mock 24 h=3 experiments). Statistical comparisons were made by two-tailed Student's t-test for unpaired samples. FIG. 3E Filter trap with anti-GFP of Q69 aggregation upon LIN treatment for the indicated times. Representative of 3 independent experiments. FIG. 3F: Representative images of Q69 aggregation in stomata from cotyledons after LIN treatment. Images show Citrine (green) and chloroplast autofluorescence (red). FIG. 3G: Citrine fluorescence (green) within the chloroplast (red) of epidermal hypocotyl cells. FIG. 3H: Representative images of mesophyll cells of 7-day-old seedlings showing Q69 distribution in Col-0 and toc159 background. In FIG. 3F-3H: Scale bar: 5 μm. Images are representative of three independent experiments. FIG. 3I: Filter trap and SDS-PAGE analysis of the samples presented in h. Actin is the loading control. Representative of 2 independent experiments. FIG. 3J: Schematic model of chloroplast-mediated regulation of polyQ69. Under normal conditions, Q69 distributes homogenously surrounding the chloroplasts, while misfolded/unstructured variants are imported to the chloroplast for degradation. When chloroplast import and degradation are impaired, misfolded Q69 accumulates in the cytosol forming aggregates. FIG. 3K: Immunoblot and filter trap analysis with anti-polyQ antibody of 7-day-old wild-type (WT) plants treated with 800 μM LIN for the indicated hours. RbcL is the loading control. Representative of 3 independent experiments.

FIGS. 4A-4M. SPP (Seq ID No 4) reduces polyQ-expanded aggregation in human cells and C. elegans. FIG. 4A: Constructs for protein expression in human HEK293 cells. FIG. 4B: Images of HEK293 cells co-transfected with mRFP-Q74 (Seq ID No. 19) and either control GFP or GFP-SPP. Blue: cell nuclei (Hoechst 33342). Scale bar: 20 μm. Representative of three independent experiments. FIG. 4C: Filter trap with anti-mCherry antibody of SDS-insoluble, aggregated mRFP-Q74 in HEK293 cells. Representative of 7 independent experiments. FIG. 4D: Relative percentage values of aggregated mRFP-Q74 to samples expressing mRFP-Q74+ control GFP (mean±s.e.m., n=7 independent experiments). FIG. 4E: Western blot of HEK293 cells with anti-GFP antibody to detect control GFP (27 kDa, green arrowhead) and SPP-GFP (˜163 kDa, yellow arrowhead). Anti-mCherry and anti-polyQ-expanded antibodies were used to detect soluble mRFP-Q74 levels. β-actin is the loading control. Representative of 7 independent experiments. FIG. 4F: Relative percentage values of soluble mRFP-Q74 (corrected for β-actin loading control) to samples expressing mRFP-Q74+ control GFP (mean±s.e.m., n=7 independent experiments). FIG. 4G: Schematic model of SPP effects in preventing mRFP-Q74 aggregation. FIG. 4H: Filter trap with anti-expanded polyQ antibody of day-3 adult worms expressing neuronal polyQ67::YFP (yellow fluorescent protein). Representative of 6 independent experiments. FIG. 4I: Relative percentage values of aggregated polyQ67 levels in C. elegans upon SPP expression (Seq. ID No 3) to control Q67 (mean±s.e.m., n=6 independent experiments). FIG. 4J: Western blot of soluble polyQ67::YFP levels (detected by anti-GFP antibody) in day-3 adult worms. α-tubulin is the loading control. Representative of 6 independent experiments. FIG. 4K: Relative percentage of soluble polyQ67 levels in worms upon SPP expression (corrected for α-tubulin levels) to control Q67 (mean±s.e.m., n=6 independent experiments). FIG. 4L: Thrashing movements in day-3 adult polyQ67-expressing worms over a 30-s period (n=50 worms per condition from three independent experiments). FIG. 4M: Thrashing movements in polyQ67 and polyQ40-expressing worms over a 30-s period at the indicated days (D) of adulthood (n=50 worms per condition from two independent experiments). In FIGS. 4L-4M, the box plots represent the 25th-75th percentiles, the line depicts the median and the whiskers show the min-max values. Statistical comparisons were made by two-tailed Student's t-test for paired (FIGS. 4D, 4F, 4I, 4K) or unpaired samples (FIGS. 4L, 4M).

FIGS. 5A-5B. Q69 aggregates upon long-term LIN treatment. FIG. 5A: Prion-like domain (PrLD) amino score (dark line) was predicted using PLAAC (http://plaac.wi.mit.edu/). Protein unstructured score (light line) was predicted with IUPred3 (https://iupred.elte.hu). Light grey boxes represent the predicted transit peptide according to ChloroP 1.1. dark grey boxes indicate the longest polyQ domain found in the amino acid sequence (from top to bottom: 1) AT5G62790-DXR, 2) AT1 G04570-FBT8, 3) AT3G56650-PPD6, 4) AT2G23070-pCK2, 5) AT2G02070-IDD5, 6) HTT exon 1 polyQ69-Q69). FIG. 5B: Immunoblot analysis of Caenorhabditis elegans expressing Q19, and constitutive transgenic Arabidopsis plants expressing Q28 and Q69. Immunoblot shows that anti-polyQ antibody can recognize different polyQ repeat sizes. Representative of two independent experiments.

FIGS. 6A-6D: SPP-GFP (Seq ID No. 23) interacts with endogenous proteins of HEK293 cells. FIG. 6A: Western blot of wild-type HEK293 cells and HEK293 cells transfected with mRFP-Q74. Anti-mCherry and anti-polyQ-expanded antibodies were used to detect soluble mRFP-Q74. Both anti-mCherry and anti-polyQ-expanded antibodies detected two common bands of soluble mRFP-Q74 with different electrophoretic mobilities, that is the most intense band of ˜55 kDa and another band of ˜43 kD. β-actin is the loading control. Representative of 9 independent experiments. FIG. 6B: Volcano plot of the interactome of synthetic SPP-GFP in HEK293 cells. Graph represents the −log 10 (P-value) of a two-tailed t-test plotted against the log 2 fold change of protein label-free quantification (LFQ) values from co-immunoprecipitation experiments using anti-GFP antibody. HEK293 cells expressing SPP-GFP were compared with HEK293 cells expressing control GFP. Red dots indicate significant interactors of SPP-GFP when compared to control GFP after correction for multiple testing. Student's t-test (n=3 biological replicates), False Discovery Rate (FDR)-adjusted P value (q value)<0.05 was considered significant. FIG. 6C: Chymotrypsin-like proteasome activity in HEK293 cells (relative slope to control GFP HEK293 cells). Graph represents the mean±s.e.m. of three independent experiments. All the statistical comparisons were made by two-tailed Student's t-test for unpaired samples. FIG. 6D: Western blot of HEK293 cells with anti-LC3 antibody to monitor autophagy flux. LC3-I is conjugated to phosphatidylethanolamine to form LC3-II, which amounts reflect the number of autophagosomes and autophagy-related structures. As a control, we treated wild-type HEK293 cells with 250 nM bafilomycin A (8 h), an inhibitor of autophagy that greatly increases the amount of LC3-II. β-actin is the loading control. Representative of three independent experiments.

FIGS. 7A-7B. Besides reducing polyQ-expanded aggregation, SPP expression leads to changes in the total levels of different proteins in Q67-expressing worms. FIGS. 7A-7B: Gene Ontology Biological Process (GOBP) analysis of FIG. 7A) downregulated, and FIG. 7B) upregulated, proteins respectively, in 3-day adult Q67::YFP worms+SPP compared with control Q67::YFP worms (FDR <0.05, Analysis tool: PANTHER Gene Ontology Resource, release 2023-06-11). Ten of the most enriched GOBP terms are shown (Fold enrichment 0-100).

FIG. 8 scheme of the used worm model for the analysis how plants prevent toxic protein aggregation. We expressed an aggregation-prone human huntingtin (Q69) fragment in Arabidopsis thaliana. In contrast to C. elegans and mammalian transgenic models, we found that Arabidopsis plants suppress toxic Q69 aggregation and do not display adverse or harmful effects. Proteomic experiments identified the chloroplast stromal processing peptidase (SPP) as one of the strongest interactors of Q69 in plant cells. Using synthetic biology and ectopically expressing SPP in human HEK293 cells and C. elegans disease models, we reduced the toxic aggregation of polyQ-extended proteins.

FIG. 11A-B. FIG. 11A: Schematic representation of constructs for protein expression in human HEK293 cells. The anti-aggregation activity of human derived SPP-like proteins such as the subunits of the MPP is going to be tested. FIG. 11B: Similar to the monomeric SPP, we proposed the creation of a monomeric human MPP where the alfa and beta subunits will be encoded by one single gene and the mitochondrial transit peptide removed to assure abrogation of import into mitochondria or chloroplast.

FIGS. 12A-12B. FIG. 12A: Alignment from Foldseek between the amino acid sequence (Seq ID No. 30) of SPP isoform 1 (AT5G42390.1) residues 1004 to 1263 (top line) and of the amino acid sequence (Seq ID No. 30) of the mitochondrial processing subunit alpha (PMPCA) (bottom line). Identical amino acids are highlighted with bold indicate sequence conservation. Gaps are represented by dashes. FIG. 12B: Superposed 3D structures of our query protein SPP (shown in grays) and the significant hit from the protein structure database PMPCA (shown in black) with a TM-Score of 0.62539 and RMSD of 6.87; TM-Score: The Template Modeling Score (TM-Score) is a measure of similarity between two protein structures. A TM-Score of 1 indicates a perfect match between two structures. RMSD: The Root Mean Square Deviation (RMSD) is also shown, a measure of structural similarity between protein structures. Different parts of the sequences of Seq Id No. 30 were aligned.

FIGS. 13A-13B. FIG. 13A: Alignment from Foldseek between the amino acid sequence (Seq ID No. 29) SPP isoform 1 (AT5G42390.1) residues 122 to 707 (top line) and of the amino acid sequence (Seq ID No. 26) of the mitochondrial processing subunit beta (PMPCB) bottom line. Identical amino acids are highlighted with bold indicate sequence conservation. The Zn²⁺-binding region that corresponds to the conserved HxxEHx₇₆E aa motif is conserved are highlighted in ovals. Gaps are represented by dashes. FIG. 13B; Superposed 3D structures of our query protein SPP (shown in gray) and the significant hit from the protein structure database PMPCA (shown in black) with a TM-Score of 0.6667 and RMSD of 10.87. TM-Score: The Template Modeling Score (TM-Score) is a measure of similarity between two protein structures. A TM-Score of 1 indicates a perfect match between two structures.

FIGS. 14A-14K. Foldseek alignment results. Alignments of amino acid sequence of SPP isoform 1 (Seq ID No. 20) and the rest of the top hits from a Foldseek alignment of SPP isoform 1 against the Homo sapiens Alphafold structure database. 1FIG. 14A: SPP (residues 95 to 995, top line) and Seq ID No 33 of Nardilysin (bottom line). FIG. 14B: SPP (residues 125 to 962, top line) and Seq ID No 34 of cDNA FLJ59785. FIG. 14C: SPP (residues 153 to 1230, top line) and Seq ID No 35 of Pitrilysin metalloproteinase 1 (bottom line). FIG. 14D: SPP (residues 134 to 762, top line) and Seq ID No 36 of Insulin-degrading enzyme (bottom line). FIG. 14E: SPP (residues 315 to 1230, top line) and Seq ID No 35 of Pitrilysin metalloproteinase 1 (A0A0AMRX9 (bottom line)). FIG. 14F: SPP (residues 642 to 1246, top line) and Seq ID No 37 of Insulysin variant (bottom line). FIG. 14G: SPP (residues 817 to 1246, top line) and Seq ID No 34 of FLJ58513 (bottom line) (Similar to MPP subunit beta). FIG. 14H: SPP (residues 785 to 1230, top line) and Seq ID No. 35 of Pitrilysin metalloproteinase 1 (B3KM51) (bottom line). FIG. 14I: SPP (residues 883 to 1228, top line) Uncharacterized protein DKFZp58611223. FIG. 14J: SPP (residues 121 to 374, top line) and Seq ID No 34 of cDNA FLJ59584 (Similar to MPP alpha). FIG. 14K: SPP (residues 157 to 304, top line) and Presequence protease mitochondrial (bottom line). SPP vs PMPCB (MPP subunit alfa) and vs PMPCB (MPP subunit beta) is already shown in FIGS. 12A and 13A.

### DETAILED DESCRIPTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

Every numerical range given throughout this specification will include every narrower numerical range that falls within such broader numerical range, as if such narrower numerical ranges were all expressly written herein. Applicant also contemplates ranges derived from data points and express ranges disclosed herein.

### Example

### 1. Methods

### 1.1 Plant Material and Constructs

Arabidopsis thaliana lines of Columbia-0 (Col-0) ecotype were employed, including wild-type, toc159^{58,59}, and cct8-2⁶⁰. Seeds underwent surface sterilization and germination on solid 0.5× Murashige and Skoog (MS) medium with vitamins, lacking sucrose. Plants were incubated in a growth chamber at 22° C. under long-day conditions (or otherwise indicated) and supplemented with 17-beta-estradiol (Sigma) when specified. The MG-132 (bio-techne) and LIN (Sigma) treatments were performed on liquid 0.5×MS medium. We used FIJI (ImageJ) to measure root length in 7-day-old seedlings grown on vertical agar plates.

For cloning, we used Gateway BP and LR Clonase II Enzyme mix (ThermoFisher). Q28 and Q69 genes were generated using the plasmid pEGFP-Q74⁶¹, with different polyQ lengths amplified and sequenced. These genes were subcloned into the entry vector pDONR221, then into vector pMpGWB105 (Q constructs). Primers Gw HTT ex1 Fw (Seq. ID No. 39), Gw Q74 Rv (Seq. ID No. 40) and Gw Citrine Fw (Seq. ID No. 41) have been used in this study.

Citrine-Q28 and Citrine-Q69 were amplified from pMpGWB105:Q28/Q69 plasmids and subcloned into entry vector pDONR221, then into destination vector pMDC7 (iQ constructs). Arabidopsis transgenic plants were generated through the floral dip method⁶². The 35S:Citrine-Q69 transgene was introduced into the toc159 or cct8-2 mutant background by cross-fertilization.

For flowering time experiments, plants were grown in short-day conditions, and rosette leaf numbers were counted until a visible bolt formed. Photosynthetic activity was assessed using the M-Series PAM fluorometer, with analysis conducted via ImagingWin (v.2.41 a) software (Heinz Walz GmbH). For heat shock assays, single plate containing 7-day-old wild-type, Q28 and Q69 was covered under aluminum foil at 45° C. (or 37° C.) for specified durations. Mock plate remained under control conditions covered with aluminum foil. Heat-treated plates were returned to 22° C. under light conditions. Microscopy images were captured using a Meta 710 Confocal Microscope with laser ablation 266 nm (Zeiss) using the same parameters between experiments.

### 1.2 Gene Expression Analysis

Total RNA was extracted from plant tissues using the RNeasy Plant Mini Kit (Qiagen). Subsequently, cDNA was synthetized using the qScript Flex cDNA synthesis kit (Quantabio). SYBR green real-time quantitative PCR experiments were performed with a 1:20 dilution of cDNA using a CFC384 Real-Time System (Bio-Rad). Data were analyzed with the comparative 2ΔΔCt method using the geometric mean of Ef1α and PP2A as housekeeping genes. For qPCR the following primers have been used: Ef1α Fw (Seq ID No. 44), Ef1α Rv (Seq ID No. 45), PP2A Fw (Seq ID No. 46), PP2A Rv (Seq ID No. 47), Hsc70-1 Fw (Seq ID No. 48), Hsc70-1 Rv (Seq ID No. 49), Hsp70b Fw (Seq ID No. 50), Hsp70b Rv (Seq ID No. 51), Hsp90-1 Fw (Seq ID No. 52), Hsp90-1 Rv (Seq ID No. 53) Hsp101b Fw (Seq ID No. 54), Hsp101b Rv (Seq ID No. 55).

1.3 Analysis of the Arabidopsis polyQ Proteome

The Arabidopsis proteome was obtained from UniProt and filtered to find proteins with 5 consecutive glutamine repeats and annotated chloroplast proteins. Prion-like domains were identified in selected protein sequences using PLAAC software (http://plaac.wi.mit.edu/)⁶³. A minimum length for prion-like domains (L core) was set at 60 and parameter a was set at 50. To identify intrinsically disordered regions, we used IUPred3 software (https://iupred.elte.hu/)⁶⁴.

### 1.4 Protein Expression and Purification

Chemically competent Escherichia coli BL21(DE3) cells were transformed with pGEX-6P-1 vector (GE Healthcare), carrying mtHTT-Exon1-polyQ69-Citrine (Q69-Citrine) and HTT-Exon1-Citrine (AQ-Citrine) constructs. Cultures were grown at 37° C. before protein expression was induced with 0.25 mM isopropyl 1-thioβ-D-galactopyranoside at 18° C. for 20 h. After harvesting and ultrasound sonication, lysates were centrifuged (25,000×g, 4° C., 1 h). Recombinant proteins were purified by GST affinity chromatography using a Glutathione-Sepharose® 4B column (Cytiva). Proteins were eluted with 20 mM reduced glutathione and 5 mM DTT in PBS pH 8. Then, free glutathione was removed from the protein solution by dialysis and the GST-fusion tag was removed with HRV 3C Protease, followed by another GST affinity chromatography. We assessed protein purity by SDS-PAGE, and concentrated pure fractions by spin filtration for import assays.

### 1.5 Chloroplast Isolation and Protein Import

Incubation occurred at 25° C. under light, halted at 5, 15, 30, and 60 min. Samples were stopped with ice-cold EDTA-containing buffer, centrifuged, chloroplast pellets were resuspended in 2× Leammli buffer. SDS-PAGE and immunoblotting with anti-GFP antibody assessed time points. Microscopy used the 30-min import reaction on a microscope slide. Chloroplasts were isolated from 12-day-old Arabidopsis seedlings as described65. For each 600 μl of import reaction, we used 10 million chloroplasts supplemented with 120 μl 10×HMS buffer (500 mM HEPES, 30 mM MgSO₄, 3.0 M sorbitol, pH 8.0), 12 μl 1 M gluconic acid (potassium salt), 6 μl 1 M NaHCO₃, 6 μl 20% (w/v) BSA, 30 μl 100 mM MgATP and 10 μM of Q69-Citrine or AQ-Citrine. To stop the reaction at different time points, we transferred 130 μL to a fresh tube with ice-cold import stop buffer (50 mM EDTA dissolved in 1×HMS buffer) and all the tubes were retained on ice until the time-course was completed. All samples were centrifuged (12,000×g, 30 s) and pellets containing the chloroplasts were resuspended in 25 μl of 2× Leammli buffer for western blot analysis. For microscopy imaging, we pipetted 60 μl of the 30-min import reaction on a microscope slide.

### 1.6 HEK293 Cell Transfection

CMV:pEGFP-Q74 plasmid was digested (BgIII, BamHI) to remove Q74 gene and generate pEGFP (CMV:GFP). SPP isoform 1 (AT5G42390, Seq ID No. 1, aa sequence Seq ID No. 20) gene, codon optimized, lacking chloroplast transit peptide, was made by Twist Bioscience. Alternatively, the same is feasible with SPP isoform 2 (Seq ID No. 2 encoding for SPP isoform 2 as shown in Seq ID No. 21). CMV:GFP-SPP (aa sequence shown in Seq ID No. 23, encoded by Seq ID No. 4) was generated by cloning the SPP gene into pDEST-CMV-N-GFP vector using Gateway technology.

HEK293 cells (ATCC, HEK293T/17, CRL-11268) were cultured on gelatin-coated plates in DMEM supplemented with 10% FBS and 1% MEM non-essential amino acids (Gibco) at 37° C. The day after seeding, HEK293 cells were transfected with 1 μg of CMV:mRFP-Q74⁴⁰ together with CMV:GFP-SPP or CMV:GFP constructs. DNA was incubated at 80° C. for 5 min and mixed with FuGENE HD (Promega) in a 3:1 ratio (FuGENE:DNA) and 65 μl of Opti-MEM (ThermoFisher) were added. The mixture was added to cells dropwise and cells were harvested for experiments after 72 h of incubation with refreshed DMEM. For microscopy, cells on coverslips were fixed with 4% PFA, and mounted for analysis with Imager Z1 microscope (Zeiss).

1.7 C. elegans Strains and Constructs

C. elegans were cultured on nematode growth media seeded with E. coli (OP50) bacteria66. As an invertebrate model organism, no ethical approval was required for work on C. elegans. Worms were examined at the adulthood ages specified in the figure legends. For all the experiments, we used hermaphrodite worms. For motility assays, worms were transferred to M9 buffer. After 30 s of adaptation, body bends were counted for 30 s. A body bend was defined as a change in mid-body bend direction.

To construct the SPP C. elegans expression plasmid, pPD95.77 from the Fire Lab kit was digested with SphI and XmaI to insert 3.6 KB of the sur5 promoter. The resultant vector was then digested with KpnI and EcoRI to excise GFP and insert a multi-cloning site containing KpnI, NheI, NotI, XbaI and EcoRI. SPP was PCR-amplified from the GFP-SPP (HEK cells) Seq ID No. 4 and cloned into the vector with NheI and NotI sites (Seq ID No. 42 and 43). The construct Seq ID No. 3 encoding for aa sequence of Seq ID No. 22 was sequence verified.

AM716 (rmls284[F25B3.3p::Q67::YFP]), AM101 (rmls110[F25B3.3p::Q40::YFP]) and AM23 (rmls298[F25B3.3p::Q19::CFP]) strains were provided by R.I. Morimoto²⁷. For the generation of DVG343 (rmls284[F25B3.3p::Q67::YFP], ocbEx277[sur-5p::SPP, myo-3p::GFP]) and DVG347 (rmls110[F25B3.3p::Q40::YFP], ocbEx279[sur-5p::SPP, myo-3p::GFP]), a DNA mixture containing 50 ng μl-1 of the plasmids sur5-p::SPP and 20 ng μl-1 pPD93 97 (myo3-p::GFP) was injected into the gonads of either adult AM716 or AM101 hermaphrodite animals using standard methods67. The corresponding control strains DVG330 (rmls284[F25B3.3p::Q67::YFP], ocbEx165[myo-3p::GFP]) and DVG346 (rmls110[F25B3.3p::Q40::YFP], ocbEx278[myo-3p::GFP]) were generated by microinjecting AM716 and AM101 worms with 20 ng μl-1 pPD93 97. The constructs comprising Q40 and Q67.

### 1.8 Filter Trap and SDS-PAGE Analysis

Plant tissues were lysed with native lysis buffer (300 mM NaCl, 100 mM Hepes pH 7.4, 2 mM EDTA, 2% Triton X-100) supplemented with plant protease inhibitor (Merck). HEK293 cells were collected in non-denaturing lysis buffer buffer (50 mM Hepes pH 7.4, 150 mM NaCl, 1 mM EDTA, 1% Triton X-100) supplemented with EDTA-free protease inhibitor cocktail (Roche). Human cells were homogenized by passing 10 times through a 27 G needle. For filter trap analysis of C. elegans, we collected day-3 adult worms with M9 buffer. Worm extracts were obtained using glass-bead disruption in non-denaturing lysis buffer (50 mM Hepes pH 7.4, 150 mM NaCl, 1 mM EDTA, 1% Triton X-100) supplemented with EDTA-free protease inhibitor cocktail. Cellular debris was removed by 2-3 centrifugation steps at 8,000×g for 5 min at 4° C. Then, we collected the supernatants and measured protein concentration with Pierce BCA Protein Assay Kit (ThermoFisher). 100 μg of protein extract was supplemented with SDS at a final concentration of 0.5%. Then, the protein extract was loaded and filtered through a cellulose acetate membrane filter (GE Healthcare Life Sciences) in a slot blot apparatus (Bio-Rad) coupled to a vacuum system. The membrane was washed with 0.2% SDS and protein aggregates were assessed by immunoblotting with either anti-GFP (AMSBIO, TP401, 1:5,000), anti-polyQ (Merck, MAB1574, clone 5TF1-1C2, 1:1,000) or anti-mCherry [1:5,000] (Abcam, ab167453, 1:5,000) as indicated in the corresponding figure legends. As secondary antibodies, we used IRDye 8000W Donkey Anti-Mouse IgG (H+L) (Licor, 926-32212, 1:10,000) and RDye 8000W Donkey anti-Rabbit IgG (H+L) (Licor, 926-32213, 1:10,000). The extracts were also analyzed by SDS-PAGE/western blot with anti-GFP (AMSBIO, TP401, 1:5,000), anti-polyQ (Merck, MAB1574, clone 5TF1-1C2, 1:1,000), anti-mCherry [1:5,000] (Abcam, ab167453, 1:5,000), anti-LC3 (Sigma, L7543, 1:1,1000), anti-β-actin (Abcam, ab8226, clone mAbcam 8226, 1:5,000) and anti-α-tubulin (Sigma-Aldrich, T6199, 1:5,000) as indicated in the figures. For western blot, we used Donkey Anti-Mouse HRP (Jackson ImmunoResearch, 715-035-150, 1:10,000) and Donkey Anti-Rabbit HRP (Jackson ImmunoResearch, 711-035-152, 1:10,000) secondary antibodies.

### 1.9 Western Blot Analysis of Plants

Plant material was grinded in liquid N2. The powder was resuspended on ice-cold TKMES homogenization buffer (100 mM Tricine-potassium hydroxide pH 7.5, 10 mM KCl, 1 mM MgCl2, 1 mM EDTA, and 10% [w/v] Sucrose) supplemented with 0.2% (v/v) Triton X-100, 1 mM DTT, 100 μg/ml PMSF, 3 μg/ml E64, and plant protease inhibitor. After centrifugation at 10,000×g for 10 min (4° C.), supernatant was collected for a second centrifugation. Protein concentration was determined with Pierce Coomassie Plus (Bradford) Protein-Assay kit. Total protein was SDS-PAGE separated, transferred to nitrocellulose membrane, and subjected to immunoblotting. The following antibodies were used for plant extracts: anti-GFP (AMSBIO, TP401, 1:5,000), anti-plant actin (Agrisera, AS132640, 1:5,000), anti-polyQ (Merck, MAB1574, clone 5TF1-1C2, 1:1,000), anti-Hsp90-1 (Agrisera, AS08346, 1:3,000), anti-Hsp70 (Agrisera, AS08371, 1:3,000), and anti-ATG8 (Agrisera, AS142769, 1:3,000).

### 1.10 Proteasome Activity

HEK293 cells were collected in proteasome activity assay buffer (50 mM Tris-HCl, pH 7.5, 10% glycerol, 5 mM MgCl₂, 0.5 mM EDTA, 2 mM ATP and 1 mM DTT) and lysed by passing 10 times through a 27 G needle attached to a 1 ml syringe. Then, we centrifuged the samples (10,000×g, 4° C., 10 min) and collected the supernatants. Protein concentrations were determined with BCA protein assay (ThermoFisher). To measure chymotrypsin-like proteasome activity, 25 μg of total protein were transferred to a 96-well microtiter plate (BD Falcon) and incubated with the fluorogenic proteasome substrate Z-Gly-Gly-Leu-AMC (Enzo). Fluorescence accumulation over time upon degradation of the proteasome substrate (380 nm excitation, 460 nm emission) was measured with a microplate fluorometer (EnSpire, Perkin Elmer) every 5 minutes for 1 hour at 37° C.

### 1.11 Interactome Analysis

Seven-day-old Q28 and Q69 seedlings were lysed in lysis buffer (1% Triton X-100, 50 mM Tris-HCl pH 8.0) supplemented with 1× plant protease inhibitor cocktail and 25 mM N-ethylmaleimide. Samples were vortexed, centrifuged at 13,000×g (10 min, 4° C.), and supernatants collected. HEK293 cells were lysed in modified RIPA buffer (50 mM Tris-HCl pH 7.4, 150 mM NaCl, 0.25% sodium deoxycholate, 1% IgPal, 1 mM PMSF, 1 mM EDTA) with protease inhibitor (Roche). Human cell lysates were centrifuged at 10,000×g (10 min, 4° C.), and supernatants collected. For each sample, the same amount of total protein was incubated for 1 hour with either anti-GFP antibody (AMSBIO, TP401, 1:500 for plants, 1:100 for HEK293) or negative control anti-IgG antibody (plants: Abcam, ab46540, 1:500; HEK293: Cell Signaling, 2729S, 1:100). Samples were then incubated with 50 μl μMACS Micro Beads (Miltenyi) for 1 hour at 4° C., loaded onto pre-cleared μMACS column (#130-042-701), and subjected to three washes using wash buffer 1 (50 mM Tris-HCl pH 7.4, 150 mM NaCl, 5% glycerol, 0.05% Triton (plants) or 0.05% IgPal (HEK293)). Next, columns were washed five times with wash buffer 2 (50 mM Tris-HCl (pH 7.4), 150 mM NaCl). Columns underwent in-column tryptic digestion with 7.5 mM ammonium bicarbonate, 2 M urea, 1 mM DTT, and 5 ng ml-1 trypsin. Digested peptides were eluted using 50 μl elution buffer 1 (2 M urea, 7.5 mM Ambic, 15 mM chloroacetamide) and incubated overnight at room temperature with shaking in the dark. The next day, samples were stage-tipped for label-free quantification.

For plant sample data acquisition, we used a Q-Exactive Plus (ThermoScientific) mass spectrometer coupled to an EASY nLC 1200 UPLC (ThermoScientific), following the protocol detailed at: https://www.ebi.ac.uk/pride/archive/projects/PXD041001. Mass spectrometric raw data were processed with MaxQuant (version 1.5.3.8)⁶⁸ using default settings with Label-free quantification (LFQ) enabled. MS2 spectra were searched against the Arabidopsis thaliana Uniprot database (UP6548, downloaded 26/08/2020), including a list of common contaminants. For HEK293 data acquisition, an Orbitrap Exploris 480 mass spectrometer (ThermoScientific, granted by the German Research Foundation (DFG) under INST 1856/71-1 FUGG) equipped with FAIMSpro and coupled to a Vanquish neo (ThermoScientific) was used, as detailed at: https://www.ebi.ac.uk/pride/archive/projects/PXD044408. Mass spectrometric raw data were processed with MaxQuant (version 2.2) against a chimeric database of Uniprot human reference database (UP5640, downloaded 04.01.2023) merged with SPP-GFP sequences, enabling the match-between-runs option between replicates. All downstream analyses were carried out on LFQ values with Perseus (plants: version 1.6.2.3; HEK293: version 1.6.15)⁶⁹. Protein groups were filtered for potential contaminants and insecure identifications. Remaining IDs were filtered for data completeness in at least one group and missing values imputed by sigma downshift (0.3 σ width, 1.8 σ downshift).

### 1.12 Limited Proteolysis-Mass Spectrometry (LiP-MS)

Cells were lysed in LiP buffer (1 mM MgCI₂, 150 mM KCl, 100 mM HEPES, pH 7.4), homogenized by electro-douncer and centrifuged at 16,000×g (10 min, 4° C.). Protein concentration was measured with the Pierce BCA Protein Assay Kit (ThermoFisher). Equal amounts of lysates were divided into PCR tube strips for LiP and control total levels proteome analysis. The samples were incubated at 25° C. for 5 min. Subsequently, proteinase K (Sigma) was added to LiP samples to a final concentration of 0.1 μg/μl, incubated at 25° C. for 5 min and then incubated at 99° C. for 5 min. Finally, the samples were incubated at 4° C. for 5 min. The control samples without proteinase K were subjected to the same incubation procedure. After that, 10% sodium deoxycholate (DOC) was added and samples were incubated on ice for 5 min. The samples were reduced using 5 mM dithiothreitol for 30 min at 37° C., followed by alkylation with 20 mM iodoacetamide (IAA) for 30 min. Then, we diluted the DOC concentration to 1% and added 1 μg trypsin together with 0.1 μg Lys-C to each sample followed by overnight incubation at 37° C. The enzymatic digestion was stopped by adding formic acid and the precipitated DOC was removed through filtration on 0.2 μm PVD membranes by spinning. Stage tip extraction was used for cleaning up peptides.

Data acquisition was performed on Orbitrap Exploris 480 mass spectrometer as detailed at: https://www.ebi.ac.uk/pride/archive/projects/PXD044409. Raw measurements were aggregated to peptide and protein quantities by DIA-NN. Structural effects were calculated using the R package LiPAnalyzeR (https://github.com/beyergroup/LiPAnalyzeR). Differential expression of peptide and protein levels was calculated using linear models where the condition is the predictor and expression is the response variable. P values of structural and expression changes were adjusted using False Discovery Rate (FDR) correction. In addition to global, i.e. within effect group correction, peptide-level effects were alternatively corrected per protein.

1.13 Quantitative Proteomics of C. elegans

Synchronized 3-day adult C. elegans were lysed in urea buffer (8 M urea, 2 M thiourea, and 10 mM Hepes (pH 7.6)) through glass-bead disruption. Following this, the samples were cleared by centrifugation at 18,000×g for 10 min. The supernatant was collected and protein concentration measured with the Pierce BCA Protein Assay Kit. The samples underwent a reduction process using 5 mM dithiothreitol for 1 h, followed by alkylation with 40 mM chloroacetamide for 30 min. Urea concentration was then reduced to 2 M, and trypsin was added at a 1:100 (w/w) ratio for overnight digestion. The next day, samples were cleared by acidification and centrifugation at maximum speed for 5 min. Stage tip extraction was employed for peptide cleanup.

Data acquisition was performed on Orbitrap Exploris 480 mass spectrometer, as outlined in detail at: https://www.ebi.ac.uk/pride/archive/projects/PXD044145. Then, samples were analyzed in DIA-NN 1.8.170. A Uniprot C. elegans canonical database (UP1940, downloaded 04/01/23) merged with the sequences of the Q67::YFP construct was used for library building. Data The DIA-NN output was further filtered based on library q-value and global q-value (s 0.01), along with a requirement of at least two unique peptides per protein, using R (4.1.3). LFQ values were computed using the DIA-NN R-package (https://github.com/vdemichev/Diann-repackage)70. Subsequent analysis was carried out using Perseus 1.6.15⁶⁹ by filtering for data completeness in at least one replicate group, followed by FDR-controlled t-tests. Gene Ontology Biological Process (GOBP) enrichment was performed with PANTHER Gene Ontology Resource (release 2023-06-11).

TABLE 2
Additional information to the used sequences as shown in the Seq ID list

| Seq | | | | |
|---|---|---|---|---|
| ID | | | | |
| No. | Name | database code/Ref | modification | comments |
| 1 | SPP, STROMAL | Tair Locus: | — | Open reading frame of |
| | PROCESSING | AT5G42390.1 | | SPP DNA sequence |
| | PEPTIDASE | | | from Arabidopsis |
| | | | | thaliana (AT5G42390.1/ |
| | | | | Isoform 1) |
| 2 | SPP, STROMAL | Tair Locus: | — | Open reading frame of |
| | PROCESSING | AT5G42390.2 | | SPP DNA sequence |
| | PEPTIDASE | | | from Arabidopsis |
| | | | | thaliana (AT5G42390.2/ |
| | | | | Isoform 2) |
| 3 | SPP (C. elegans | — | Deletion of | SPP syntehtic DNA |
| | expression) | | 54 | sequence for C. |
| | | | aminoacids | elegans, codons were |
| | | | corresponding | optimized for human |
| | | | to the N- | expression ///// Used for |
| | | | terminal | the experiments with |
| | | | chloroplast | worms, a part of the N- |
| | | | transit | terminal (chloroplast |
| | | | peptide. A | transit peptide) was |
| | | | methione | deleted |
| | | | was added | |
| | | | as start | |
| | | | codon | |
| 4 | GFP-SPP (Hek cells | — | GFP was | GFP sequence and |
| | expression) | | fused to the | SPP DNA sequence for |
| | | | N-terminal | HEK cells //// Used for |
| | | | of SPP. SPP | the experiments with |
| | | | has a | HEK cells, a part of the |
| | | | deletion of | N-terminal (chloroplast |
| | | | 54 | transit peptide) was |
| | | | aminoacids | deleted from original |
| | | | corresponding | reference sequence |
| | | | to the N- | isoform 1. GFP is fused |
| | | | terminal | to the N-terminal |
| | | | chloroplast | |
| | | | transit | |
| | | | peptide. No | |
| | | | methione | |
| | | | added as | |
| | | | the start | |
| | | | codon | |
| | | | comes from | |
| | | | GFP. | |
| 5 | MPPB_HUMAN | GenBank: AF054182.1 | N-ter signal | The domain Peptidase |
| | Mitochondrial- | | peptide was | M16, N-terminal of SPP |
| | processing peptidase | | removed | has a 33.64% amino |
| | subunit beta OS = Homo | | and a new | acid sequence identity |
| | sapiens OX = 9606 | | start codon | with the mitochondrial |
| | GN = PMPCB PE = 1 | | was added | processing peptidase |
| | SV = 2 | | | beta-subunit from |
| | | | | human |
| 6 | PREDICTED: Homo | NCBI Reference | N-ter signal | PREDICTED: Homo |
| | sapiens peptidase, | Sequence: | peptide was | sapiens peptidase, |
| | mitochondrial | XM_005266059.4 | removed | mitochondrial |
| | processing subunit | | and a new | processing subunit |
| | alpha (PMPCA), | | start codon | alpha (PMPCA), |
| | transcript variant X1, | | was added | transcript variant X1, |
| | mRNA | | | mRNA. |
| 7 | MPPB_HUMAN | GenBank: AF054182.1 | — | The domain Peptidase |
| | Mitochondrial- | | | M16, N-terminal of SPP |
| | processing peptidase | | | has a 33.64% amino |
| | subunit beta OS = Homo | | | acid sequence identity |
| | sapiens OX = 9606 | | | with the mitochondrial |
| | GN = PMPCB PE = 1 | | | processing peptidase |
| | SV = 2 | | | beta-subunit from |
| | | | | human |
| 8 | PREDICTED: Homo | NCBI Reference | — | PREDICTED: Homo |
| | sapiens peptidase, | Sequence: | | sapiens peptidase, |
| | mitochondrial | XM_005266059.4 | | mitochondrial |
| | processing subunit | | | processing subunit |
| | alpha (PMPCA), | | | alpha (PMPCA), |
| | transcript variant X1, | | | transcript variant X1, |
| | mRNA | | | mRNA. |
| 9 | The conserve glycine- | — | — | The DNA coding |
| | rich loop | | | sequence of the |
| | “GGGGSFSAGGPGKGMFS” | | | conserve glycine-rich |
| | | | | loop |
| | | | | “GGGGSFSAGGPGKGMFS” |
| | | | | which is essential |
| | | | | for substrate biding of |
| | | | | the alfa subunit of the |
| | | | | MPP (Also known as |
| | | | | PMPCA). Based in the |
| | | | | transcript PMPCA-201 |
| | | | | (ENST00000371717) |
| 10 | SPP sequence residues | Truncated version of SPP | Truncated | SPP sequence residues |
| | 122 to 707 | (AT5G42390.1) residues | version of | 122 to 707 that aligns |
| | | 122 to 707 | SPP | with HUMAN |
| | | | (AT5G42390.1) | Mitochondrial- |
| | | | residues | processing peptidase |
| | | | 122 to 707 | subunit beta |
| 11 | SPP sequence residues | Truncated version of SPP | Truncated | SPP sequence residues |
| | 1004 to 1263 | (AT5G42390.1) residues | version of | 1004 to 1263 that aligns |
| | | 1004 to 1263 | SPP | with mitochondrial |
| | | | (AT5G42390.1) | processing subunit |
| | | | residues | alpha (PMPCA) |
| | | | 1004 to | |
| | | | 1263 | |
| 12 | Zn2+-binding region | Zn2+-binding region | Trucanted | the Zn2+-binding region |
| | (HxxEHx76E motif) | (HxxEHx76E motif) from | version of | that corresponds to the |
| | from SPP | SPP (AT5G42390.1) | SPP | conserved HxxEHx76E |
| | | | containing | motif is conserved in |
| | | | the Zn2+- | the SPP and the in the |
| | | | binding | MPP β subunit. Any |
| | | | region | mutation of any of these |
| | | | (HxxEHx76E | residues eliminates |
| | | | motif) | Zn2+ binding and |
| | | | | blocks the peptidase |
| | | | | activity. |
| 13 | Zn2+-binding region | Zn2+-binding region | Trucanted | the Zn2+-binding region |
| | (HxxEHx76E motif) | (HxxEHx76E motif) from | version of | that corresponds to the |
| | from HUMAN | HUMAN Mitochondrial- | Zn2+- | conserved HxxEHx76E |
| | Mitochondrial- | processing peptidase | binding | motif is conserved in |
| | processing peptidase | subunit beta | region | the SPP and the in the |
| | subunit beta | | (HxxEHx76E | MPP β subunit. Any |
| | | | motif) | mutation of any of these |
| | | | from | residues eliminates |
| | | | HUMAN | Zn2+ binding and |
| | | | Mitochondrial- | blocks the peptidase |
| | | | processing | activity. |
| | | | peptidase | |
| | | | subunit beta | |
| 14 | Nardilysin (NRD1) | GenBank: AY049784.1 | — | Homo sapiens |
| | | | | sequence |
| 15 | cDNA FLJ59785, highly | GenBank: AK302616.1 | — | Homo sapiens |
| | similar to Nardilysin | | | sequence |
| 16 | Pitrilysin | Transcript: | — | Homo sapiens |
| | metalloproteinase 1 | ENST00000678987.1 | | sequence |
| | | PIT RM1-237 | | |
| 17 | Insulin-degrading | Transcript: | — | Homo sapiens |
| | enzyme | ENST00000678844.1 | | sequence |
| | | IDE-230 | | |
| 18 | Insulin-degrading | | — | Homo sapiens |
| | enzyme | | | sequence/short |
| | | | | version |
| 19 | mRFP-Q74 | — | mRFP fused | Modified from pEGFP- |
| | | | to fragment | Q74 (Addgene Plasmid |
| | | | of the | #40262)/Used for the |
| | | | Huntingtin | transfection |
| | | | exon 1 with | experiments in HEK |
| | | | 74 | cells |
| | | | glutamine | |
| | | | repetitions | |
| 20 | SPP, STROMAL | Uniprot: A0A654G7G2/ | — | SPP amino acid |
| | PROCESSING | Tair Locus: AT5G42390.1 | | sequence from |
| | PEPTIDASE | | | Arabidopsis thaliana |
| | | | | (A0A654G7G2/ |
| | | | | AT5G42390.1/Isoform |
| | | | | 1) |
| 21 | SPP, STROMAL | Uniprot: A0A1P8BEG1/ | — | SPP aminoacid |
| | PROCESSING | Tair Locus: AT5G42390.2 | | sequence from |
| | PEPTIDASE | | | Arabidopsis thaliana |
| | | | | (A0A1P8BEG1/ |
| | | | | AT5G42390.2/Isoform |
| | | | | 2) |
| 22 | SPP (C. elegans | — | Deletion of | SPP aminoacid |
| | expression) | | 54 | sequence for C. |
| | | | aminoacids | elegans ///// Used for |
| | | | corresponding | the experiments with |
| | | | to the N- | worms, a part of the N- |
| | | | terminal | terminal (chloroplast |
| | | | chloroplast | transit peptide) was |
| | | | transit | deleted based on |
| | | | peptide. A | original reference |
| | | | methione | sequence isoform 1 |
| | | | was added | |
| | | | as start | |
| | | | codon | |
| 23 | GFP-SPP (Hek cells | — | GFP was | SPP aminoacid |
| | expression) | | fused to the | sequence for HEK cells |
| | | | N-terminal | (fused to GFP) //// |
| | | | of SPP. | Used for the |
| | | | SPP has a | experiments with HEK |
| | | | deletion of 54 | cells, a part of the N- |
| | | | aminoacids | terminal (chloroplast |
| | | | corresponding | transit peptide) was |
| | | | to the N- | deleted based on |
| | | | terminal | original reference |
| | | | chloroplast | sequence isoform 1. |
| | | | transit | GFP is fused to the N- |
| | | | peptide. No | terminal |
| | | | methione | |
| | | | added as | |
| | | | the start | |
| | | | codon | |
| | | | comes from | |
| | | | GFP. | |
| 24 | HUMAN Peptidase, | Uniprot: Q5SXN9 | N-ter signal | |
| | mitochondrial | | peptide was | |
| | processing subunit | | removed | |
| | alpha OS = Homo | | and a new | |
| | sapiens OX = 9606 | | start codon | |
| | GN = PMPCA PE = 1 | | was added | |
| | SV = 2 | | | |
| 25 | MPPB_HUMAN | Uniprot: O75439/Protein | N-ter signal | The domain Peptidase |
| | Mitochondrial- | sequence AAC39915.1 | peptide was | M16, N-terminal of SPP |
| | processing peptidase | | removed | has a 33.64% amino |
| | subunit beta OS = Homo | | and a new | acid sequence identity |
| | sapiens OX = 9606 | | start codon | with the mitochondrial |
| | GN = PMPCB PE = 1 | | was added | processing peptidase |
| | SV = 2 | | | beta-subunit from |
| | | | | human |
| 26 | MPPB_HUMAN | Uniprot: O75439/Protein | — | The domain Peptidase |
| | Mitochondrial- | sequence AAC39915.1 | | M16, N-terminal of SPP |
| | processing peptidase | | | has a 33.64% amino |
| | subunit beta OS = Homo | | | acid sequence identity |
| | sapiens OX = 9606 | | | with the mitochondrial |
| | GN = PMPCB PE = 1 | | | processing peptidase |
| | SV = 2 | | | beta-subunit from |
| | | | | human |
| 27 | HUMAN Peptidase, | Uniprot: Q5SXN9 | — | |
| | mitochondrial | | | |
| | processing subunit | | | |
| | alpha OS = Homo | | | |
| | sapiens OX = 9606 | | | |
| | GN = PMPCA PE = 1 | | | |
| | SV = 2 | | | |
| 28 | The conserve glycine- | — | — | The conserve glycine- |
| | rich loop | | | rich loop |
| | “GGGGSFSAGGPGKGMFS” | | | “GGGGSFSAGGPGKGMFS” |
| | | | | which is essential |
| | | | | for substrate biding of |
| | | | | the alfa subunit of the |
| | | | | MPP (Also known as |
| | | | | PMPCA) |
| 29 | SPP sequence residues | Truncated version of SPP | Truncated | SPP sequence residues |
| | 122 to 707 | (AT5G42390.1) residues | version of | 122 to 707 that aligns |
| | | 122 to 707 | SPP | with HUMAN |
| | | | (AT5G42390.1) | Mitochondrial- |
| | | | residues | processing peptidase |
| | | | 122 to 707 | subunit beta |
| 30 | SPP sequence residues | Truncated version of SPP | Truncated | SPP sequence residues |
| | 1004 to 1263 | (AT5G42390.1) residues | version of | 1004 to 1263 that aligns |
| | | 1004 to 1263 | SPP | with mitochondrial |
| | | | (AT5G42390.1) | processing subunit |
| | | | residues | alpha (PMPCA) |
| | | | 1004 to | |
| | | | 1263 | |
| 31 | Zn2+-binding region | Zn2+-binding region | Trucanted | the Zn2+-binding region |
| | (HxxEHx76E motif) | (HxxEHx76E motif) from | version of | that corresponds to the |
| | from SPP | SPP (AT5G42390.1) | SPP | conserved HxxEHx76E |
| | | | containing | motif is conserved in |
| | | | the Zn2+- | the SPP and the in the |
| | | | binding | MPP β subunit. Any |
| | | | region | mutation of any of these |
| | | | (HxxEHx76E | residues eliminates |
| | | | motif) | Zn2+ binding and |
| | | | | blocks the peptidase |
| | | | | activity. |
| 32 | Zn2+-binding region | Zn2+-binding region | Trucanted | the Zn2+-binding region |
| | (HxxEHx76E motif) | (HxxEHx76E motif) from | version of | that corresponds to the |
| | from HUMAN | HUMAN Mitochondrial- | Zn2+- | conserved HxxEHx76E |
| | Mitochondrial- | processing peptidase | binding | motif is conserved in |
| | processing peptidase | subunit beta | region | the SPP and the in the |
| | subunit beta | | (HxxEHx76E | MPP β subunit. Any |
| | | | motif) | mutation of any of these |
| | | | from | residues eliminates |
| | | | HUMAN | Zn2+ binding and |
| | | | Mitochondrial- | blocks the peptidase |
| | | | processing | activity. |
| | | | peptidase | |
| | | | subunit beta | |
| 33 | Nardilysin (NRD1) | UniProt: Q96L67 | — | Homo sapiens |
| | | | | sequence |
| 34 | cDNA FLJ59785, highly | Uniprot: B4DYV0 | — | Homo sapiens |
| | similar to Nardilysin | | | sequence |
| 35 | Pitrilysin | Uniprot: A0A712YQT2 | — | Homo sapiens |
| | metalloproteinase 1 | | | sequence |
| 36 | Insulin-degrading | Uniprot: A0A712V612 | — | Homo sapiens |
| | enzyme | | | sequence |
| 37 | Insulin-degrading | Uniprot: A0A712V634 | — | Homo sapiens |
| | enzyme | | | sequence/short |
| | | | | version |
| 38 | mRFP-Q74 | — | mRFP fused | Modified from pEGFP- |
| | | | to fragment | Q74 (Addgene Plasmid |
| | | | of the | #40262)/Used for the |
| | | | Huntingtin | transfection |
| | | | exon 1 with | experiments in HEK |
| | | | 74 | cells |
| | | | glutamine | |
| | | | repetitions | |
| 39 | Gw HTT ex1 Fw | — | — | Primers used for plant |
| | | | | expression plasmid |
| 40 | Gw Q74 Rv | — | — | Primers used for plant |
| | | | | expression plasmid |
| 41 | Gw Citrine Fw | — | — | Primers used for plant |
| | | | | expression plasmid |
| 42 | NheI SPP Fw | — | — | Primers used for C. |
| | | | | elegans expression |
| | | | | plasmid |
| 43 | NotI SPP Rv | — | — | Primers used for C. |
| | | | | elegans expression |
| | | | | plasmid |
| 44 | Ef1α Fw | — | — | Primers for qPCR |
| 45 | Ef1α Rv | — | — | Primers for qPCR |
| 46 | PP2A Fw | — | — | Primers for qPCR |
| 47 | PP2A Rv | — | — | Primers for qPCR |
| 48 | Hsc70-1 Fw | — | — | Primers for qPCR |
| 49 | Hsc70-1 Rv | — | — | Primers for qPCR |
| 50 | Hsp70b Fw | — | — | Primers for qPCR |
| 51 | Hsp70b Rv | — | — | Primers for qPCR |
| 52 | Hsp90-1 Fw | — | — | Primers for qPCR |
| 53 | Hsp90-1 Rv | — | — | Primers for qPCR |
| 54 | Hsp101b Fw | — | — | Primers for qPCR |
| 55 | Hsp101b Rv | — | — | Primers for qPCR |

### 2. Results

### Arabidopsis Prevents 069 Aggregation Under Normal Conditions

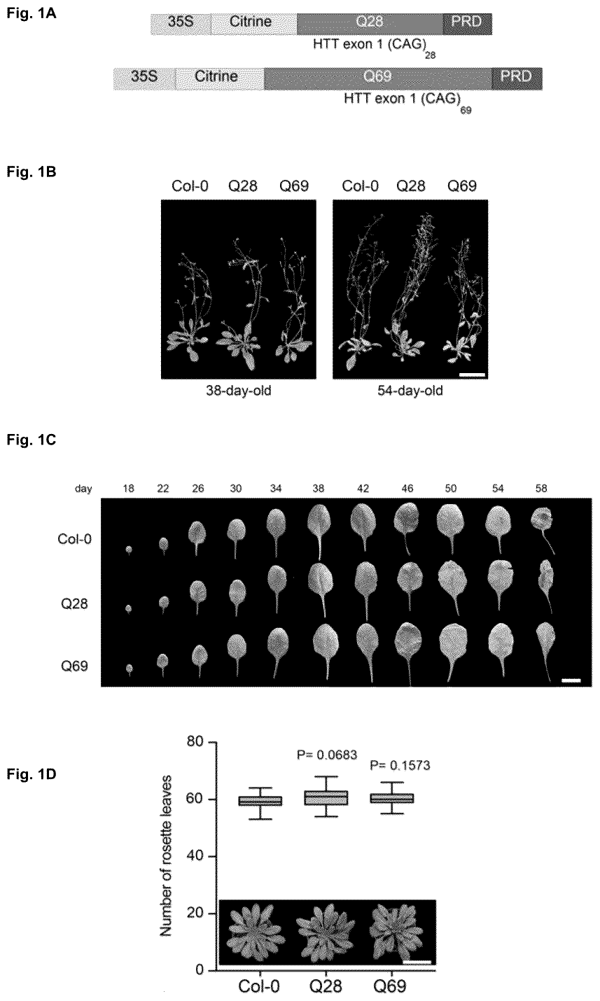

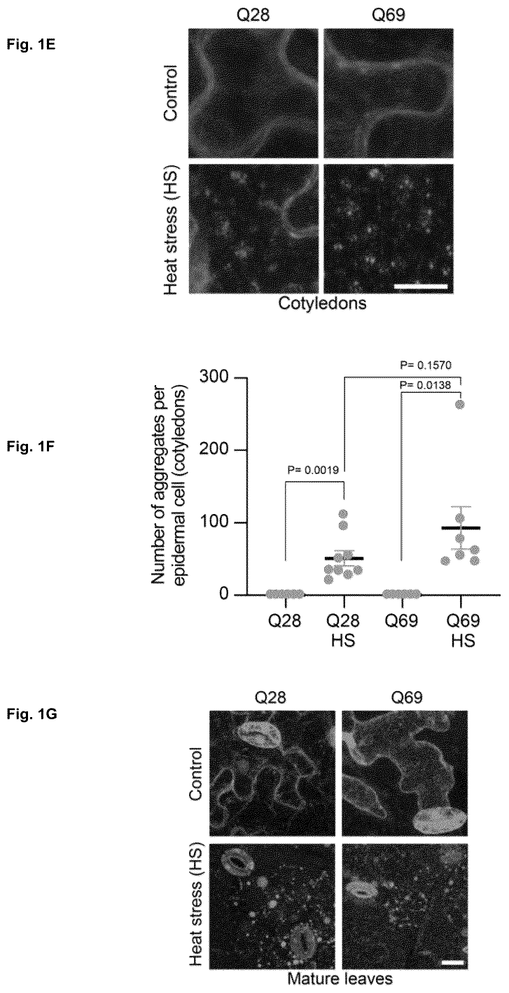

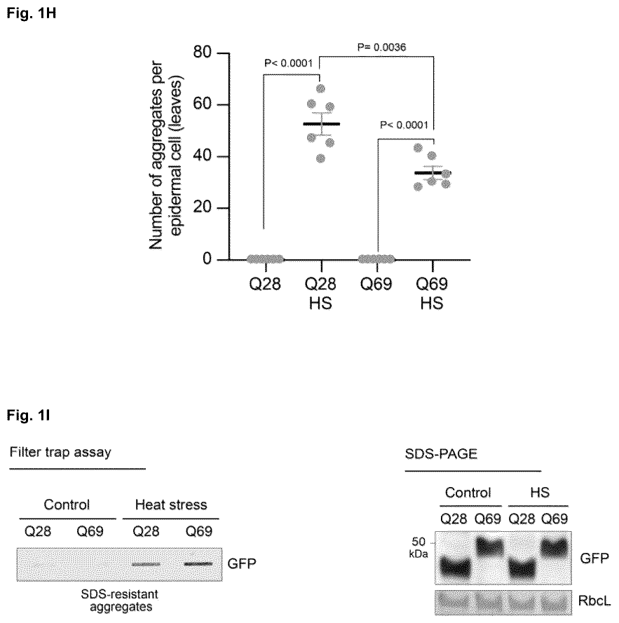

In invertebrate and mammalian model organisms, the expression of HTT exon 1 containing more than 35 glutamine repeats is sufficient to trigger polyQ aggregation6,^{25,26}. To recapitulate the pathological aggregation phenotype of Huntington's disease in plants, we generated transgenic Arabidopsis expressing the human mutant HTT exon 1 fragment. To this end, we generated the constructs 35S:Citrine-HTTexon1-Q28 (028) and 35S:Citrine-HTTexon1-Q69 (069) (FIG. 1A). Subsequently, we established and characterized Arabidopsis transgenic plants expressing 028 and 069 under the control of the 35S promoter (FIGS. 1B-1D). Constitutive expression of 028 and 069 did not cause deleterious effects in Arabidopsis plants, which exhibited similar development, lifespan, flowering time, and photosynthetic activity compared to untransformed Col-0 wild-type controls (FIGS. 1B-1D).

We observed a diffuse distribution pattern for both 028 and 069 proteins in the root tips, cotyledons, and mature leaves of plants under normal growth conditions (not shown here, published in Llamas et al 2023). Moreover, polyQ-expanded proteins did not induce proteostasis stress markers, indicating absence of proteotoxicity in these transgenic lines. To tightly control the expression of polyQ proteins, we generated inducible transgenic plants that express Q28 or Q69 in the presence of estradiol. After 7 days of estradiol treatment, we did not observe aggregation or toxic effects in either inducible Q28 or Q69 seedlings (not shown here, published in Llamas et al 2023). Together, our results indicate that Arabidopsis plants have mechanisms to sustain proteostasis and prevent polyQ aggregation throughout the plant life.

In humans, HTT and ATXN3 can contain up to 35 and 52 polyQ repeats, respectively, before becoming prone to aggregation even under stress conditions^{9,12,18}. In contrast, the polyQ stretches in endogenous Arabidopsis proteins do not exceed 24 glutamine repeats²⁰ (not shown here but published in Llamas et al 2023). Among them, ELF3 protein can form aggregates at higher temperatures even with a short polyQ7 stretch²¹. We hypothesized that, unlike animals^{26,27}, relatively shorter polyQ stretches are prone to aggregation in plants during stress conditions. Thus, plants might require intrinsic proteostasis mechanisms to avoid polyQ aggregation under normal conditions. To assess whether elevated temperatures trigger polyQ-expanded aggregation, we subjected 7-day-old stable transgenic plants expressing Q28 and Q69 to either mild (37° C.) or severe heat stress (45° C.) for 90 minutes. Although mild stress conditions did not cause aggregation of cytosolic Q28 and Q69 (not shown here, published in Llamas et al 2023), a severe heat stress led to the formation of Q28 and Q69 aggregates (FIGS. 1E-1I). However, Q28 and Q69 seedlings did not exhibit increased sensitivity to heat stress compared to wild-type plants (not shown here, published in Llamas et al 2023).

Q69 Interacts with Chloroplast Proteostasis Components

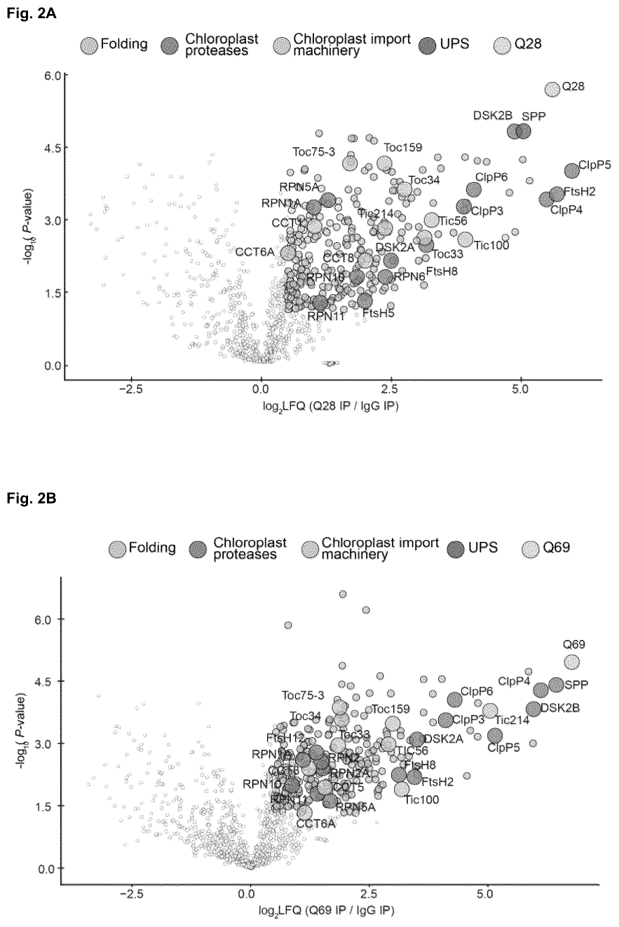

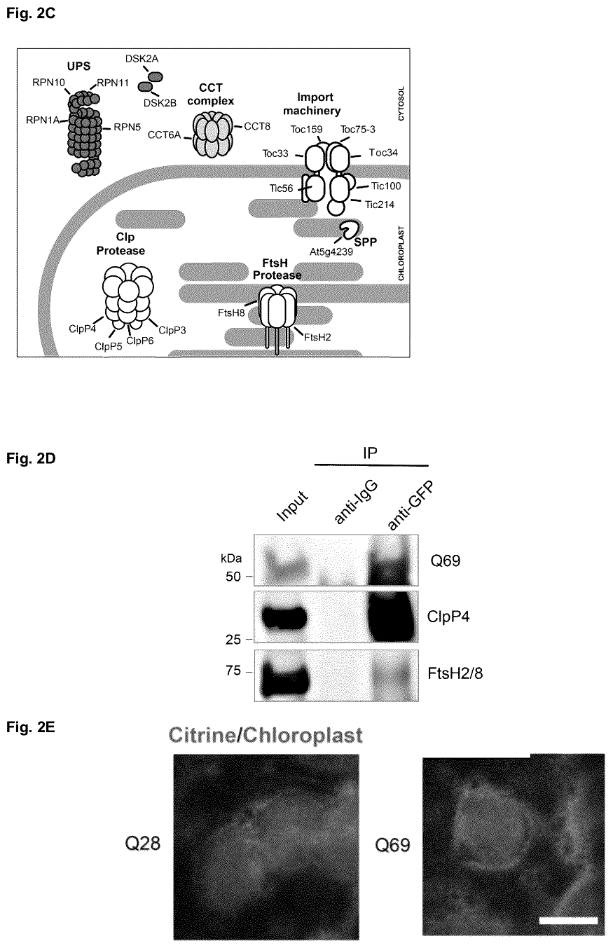

To investigate the mechanisms underlying the enhanced ability of plants to prevent polyQ aggregation under normal conditions, we performed pulldown experiments of Q28 and Q69 in Arabidopsis followed by label-free proteomics. Q28 and Q69 were the most enriched proteins in the corresponding transgenic plants after immunoprecipitation, thereby validating our assay (FIGS. 2A, 2B). Hierarchical clustering analysis revealed a similar network of interactions between Q69 and Q28 lines (not shown here, published in Llamas et al 2023). Thus, the plant proteostasis interactors did not differ between the relatively long Q28 and Q69 stretches, considering that Q24 represents the longest polyQ stretch in Arabidopsis proteins.

Among the proteins interacting with Q28 and Q69, we found several factors involved in cytosolic protein folding and the ubiquitin-proteasome system (FIGS. 2A-2C). For instance, we identified subunits of the TRiC/CCT complex (FIGS. 2A-2C), a chaperonin that reduces the accumulation of polyQ aggregates in human cells and C. elegans models^{5,10}. Moreover, we detected the ubiquitin-binding receptors DSK2A and DSK2B as interactors of Q28 and Q69 in Arabidopsis (FIGS. 2A-2C). Importantly, DSK2 (also known as Ubiquilin) suppresses polyQ-expanded protein aggregation and toxicity in animal models of Huntington's disease²⁸. Consistent with other interactome studies in mammalian cells²⁹, we identified several proteasome subunits as polyQ interactors in plants (FIGS. 2A-2C). In animal cells, proteasome inhibition leads to the aggregation of polyQ-expanded proteins⁹. Similarly, we observed Q69 aggregation when plants were exposed to proteasome inhibitor MG-132 (not shown here, published in Llamas et al 2023).

In addition to cytosolic proteostasis components, our interactome analysis revealed that polyQ proteins bind to chloroplast-specific proteins such as the stromal processing peptidase (SPP). We also found several components of TOC/TIC, the chloroplast import machinery, as well as the proteases complexes Clp and FtsH (FIGS. 2A-2D). Moreover, confocal microscopy analyses indicated that both Q28 and Q69 localize around the chloroplasts (FIG. 2E). These findings suggest a potential link between chloroplasts and polyQ proteostasis in plants.

Chloroplast Disruption Causes Cytosolic polyQ Aggregation

Most chloroplast proteins are encoded by the nuclear genome and synthesized in the cytosol as unfolded protein precursors (or pre-proteins), which are imported into chloroplasts by the TOC/TIC machinery. Pre-proteins contain an unstructured/unfolded N-terminal transit peptide^{30,31} that is recognized by the TOC/TIC complex and transported into the stroma for proteolytic processing by proteases^{32,33}. The protease complexes Clp and FtsH also degrade damaged and misfolded proteins, thus maintaining chloroplast proteostasis^{32,33}. Notably, the interactome of both Q28 and Q69 was enriched for subunits of the TOC/TIC import machinery, as well as Clp and FtsH proteases (FIGS. 2A-2C).

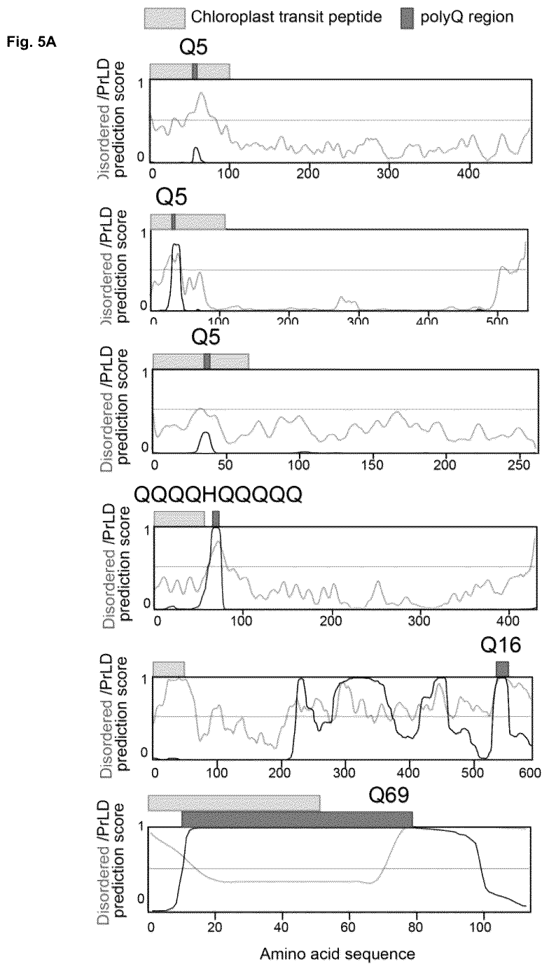

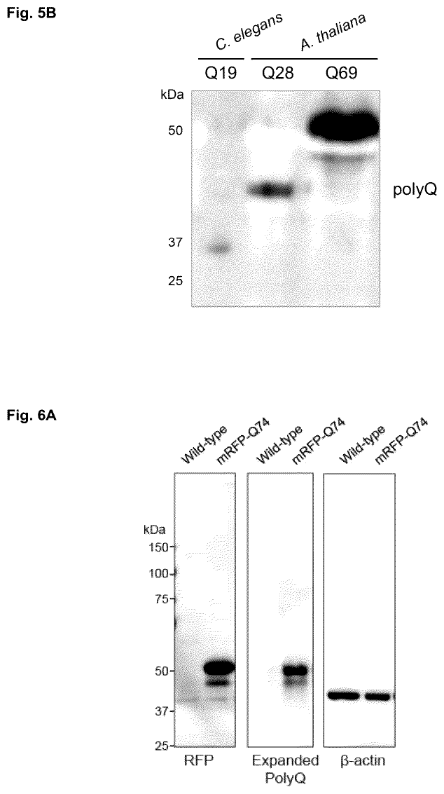

We hypothesized that polyQ proteins can be recognized by the chloroplast import machinery. First, we analyzed the endogenous Arabidopsis proteome, searching for polyQ stretches in annotated chloroplast proteins (Llamas et al 2023). From the nucleus encoded-chloroplast list of proteins with polyQ stretches, we found that 5 out of these proteins have the polyQ repeats close to the N-terminal chloroplast transit peptide (FIG. 5A). Prediction software indicated that the polyQ-stretches from chloroplast proteins are embedded in prion-like domains or intrinsically disordered regions (FIG. 5A). Likewise, the Q69 protein, which has a large prion-like/disorder domain, was also predicted to be a chloroplast protein (FIG. 5A).

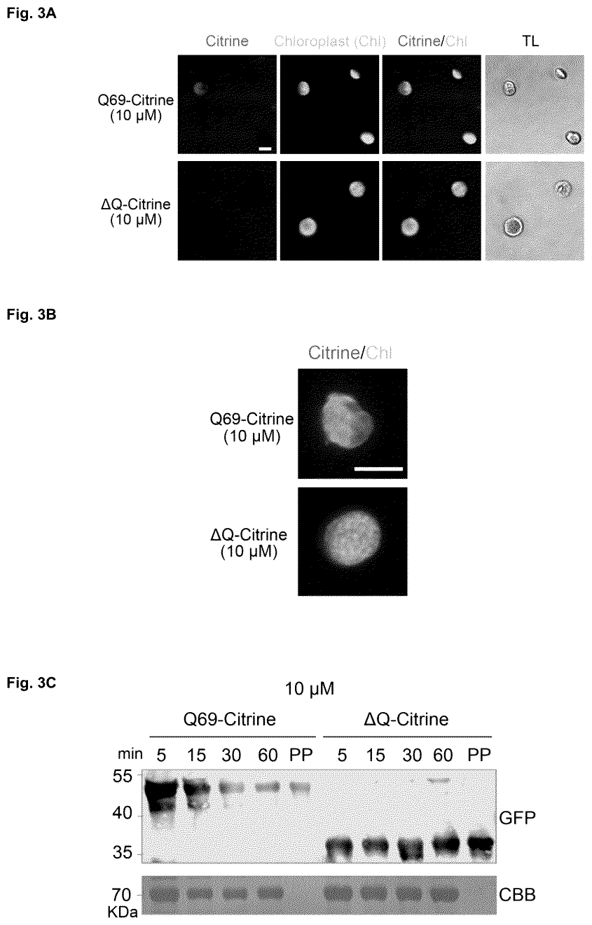

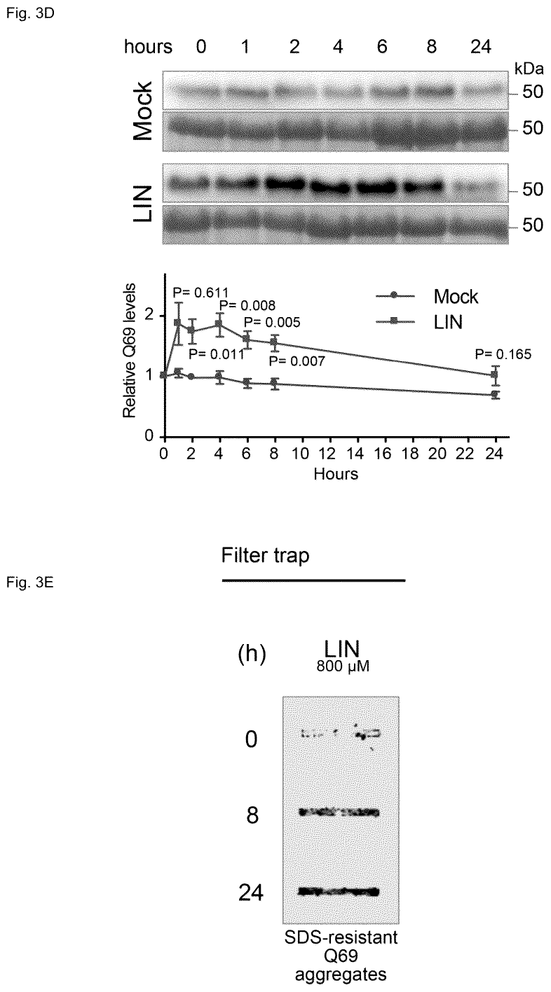

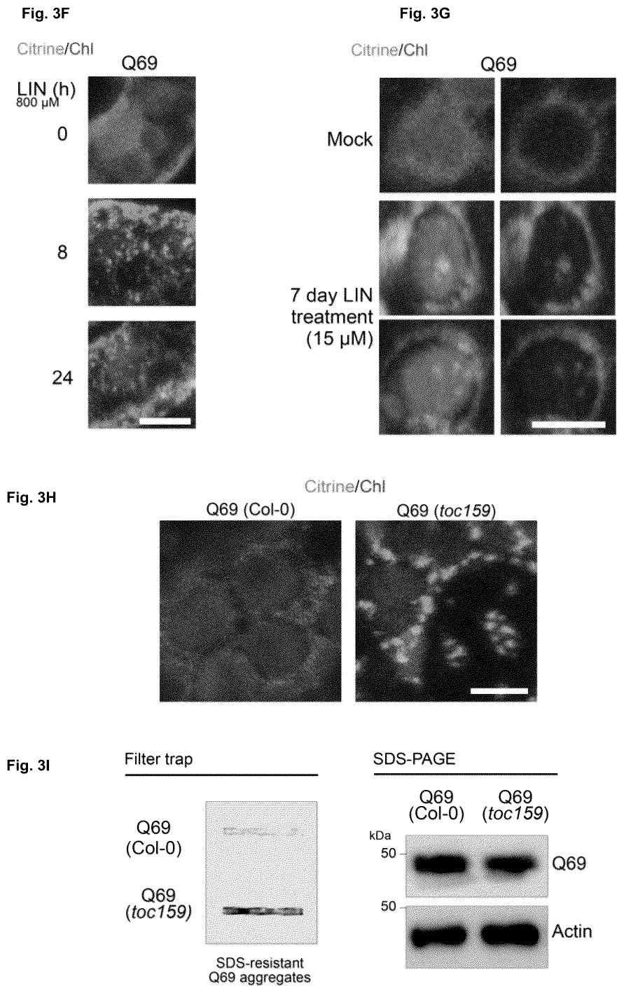

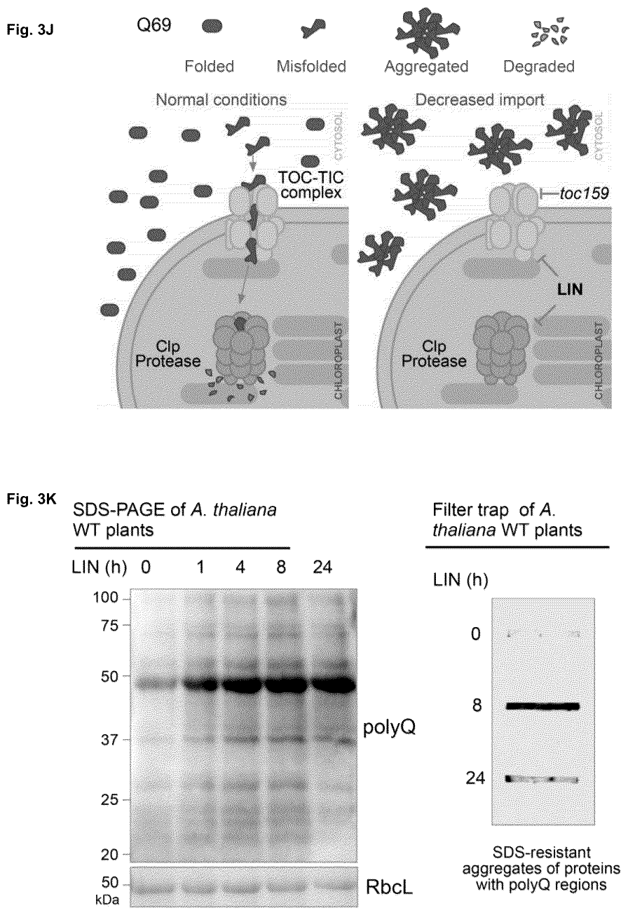

To assess whether polyQ-expanded proteins are imported and degraded within the chloroplast, we incubated isolated chloroplasts with purified recombinant polyQ69-HTTexon1 fused to the fluorescent tag Citrine (Q69-Citrine). We found that isolated chloroplasts import Q69-Citrine, but not control HTTexon1-Citrine lacking the polyQ stretch (AQ-Citrine) (FIGS. 3A, 3B). In addition, isolated chloroplasts degraded Q69-Citrine over time, whereas the levels of AQ-Citrine remained stable (FIG. 3C). To investigate the impact of chloroplasts on polyQ proteostasis in vivo, we treated plants with lincomycin (LIN), which impairs both chloroplast protein import and Clp protease-mediated degradation^{34,35}. When we transferred 7-day-old Q69 seedlings to liquid media supplemented with 800 μM LIN, we observed a rapid accumulation and aggregation of Q69 (FIGS. 3D-3F). After 24 hours of treatment with 800 μM LIN, Q69 remained aggregated but its soluble levels were reduced (FIGS. 3D-3F). These results suggest that blocking chloroplast import and Clp-mediated degradation initially increases Q69 levels, leading to its aggregation. Eventually, the prolonged aggregation induced by acute LIN treatment reduces the levels of monomeric, soluble Q69 (FIGS. 3D-3F). Notably, treating plants with lower concentrations of LIN (15 μM) for extended periods of time (7 days) also triggered Q69 aggregation (not shown here, published in Llamas et al 2023). During long-term LIN treatment, we detected Citrine fluorescence within some chloroplasts (FIG. 3G), providing further evidence that Q69 can be imported into these organelles. Similarly, we also observed Q69 aggregates in toc159 plants, a mutant line with altered chloroplast import (FIGS. 3H, 3I).

In human cells and animal models, the cytosolic TRiC/CCT chaperonin and the ubiquitin-proteasome system prevent polyQ-expanded aggregation^{5,9,10,29}. Importantly, genetic impairment of cytosolic folding through loss of the TRIC/CCT complex and prolonged proteasomal inhibition allowed us to detect Q69-Citrine fluorescence in chloroplasts as well as the formation of nuclear condensates/aggregates (not shown here, published in Llamas et al 2023). Collectively, our data suggest that Q69 can be targeted to different subcellular compartments, and chloroplasts may play a major role in preventing the accumulation of Q69 aggregates in the cytosol (FIG. 3J). Supporting this hypothesis, when chloroplasts were transiently impaired upon LIN treatment, cytosolic Q69 levels rapidly increased surpassing a threshold that triggers the formation of cytosolic aggregates (FIGS. 3D-3F).

Intrigued by the interplay between chloroplast proteostasis and the regulation of Q69 aggregation, we asked whether LIN treatment also promotes the aggregation of endogenous polyQ-proteins in Arabidopsis. To this end, we used a polyQ antibody which specifically recognizes proteins containing polyQ stretches (FIG. 5B). Remarkably, treatment of wild-type Arabidopsis plants with LIN caused a strong accumulation and aggregation of endogenous polyQ-proteins, indicating a central role of chloroplasts in polyQ proteostasis (FIG. 3K).

SPP Reduces polyQ Aggregation in Human Cells and C. elegans

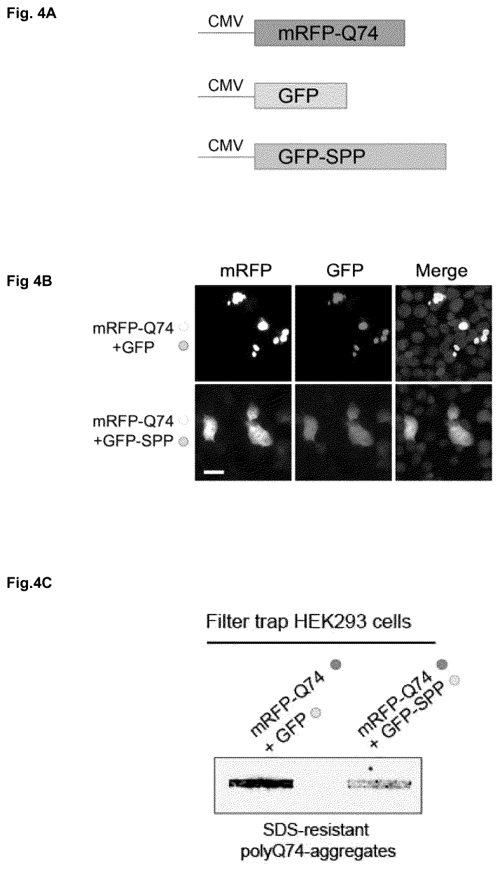

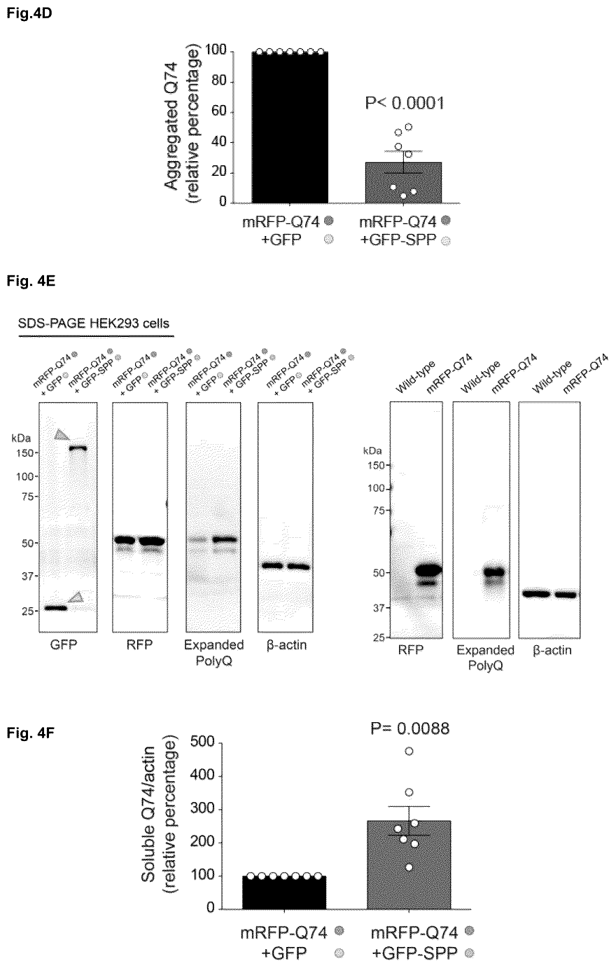

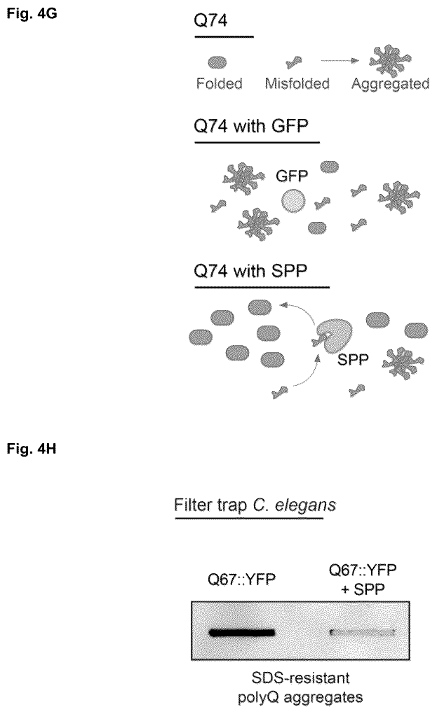

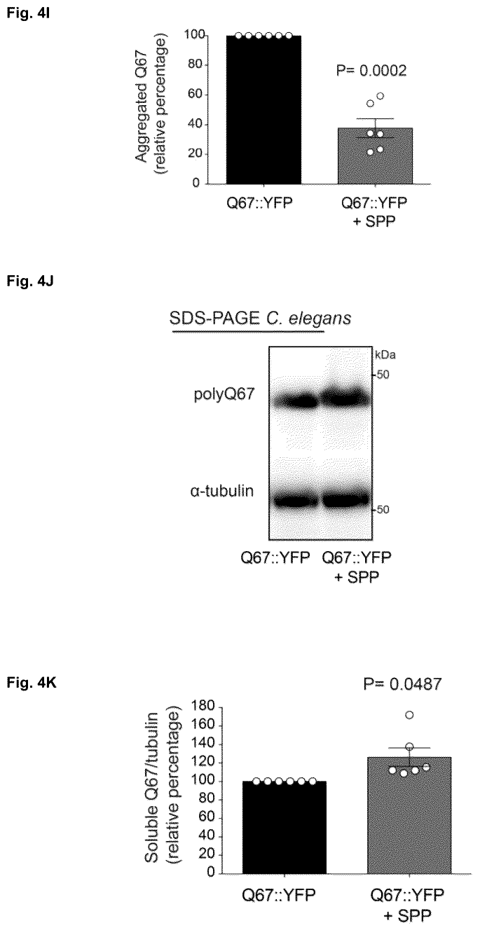

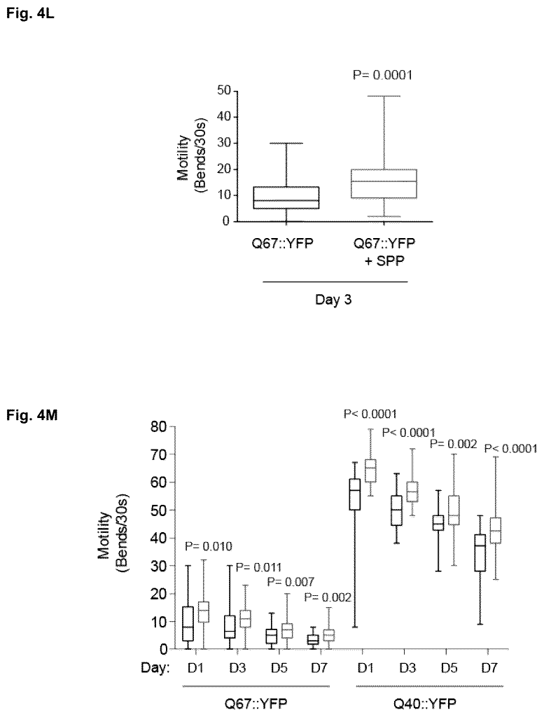

Besides Q69 itself, the stromal processing peptidase (SPP) stood out as the most enriched protein after immunoprecipitation of polyQ69 in plants (FIG. 2B). Similarly, SPP was also one of the most enriched interactors of Q28 (FIG. 2A). SPP binds to pre-proteins and cleaves their chloroplast transit peptide through a single endoproteolytic step³⁶. In addition, SPP is upregulated and binds to unstructured peptides to counteract the loss of chaperone capacity in plants, suggesting a role of SPP in preventing folding stress³⁷. To explore whether SPP can function in human cells to decrease polyQ-expanded aggregation, we co-transfected human HEK293 cells with mRFP-HTTexon1-Q74 (mRFP-Q74) and a SPP (without chloroplast transit peptide and human codon optimized) fused to GFP in the N-terminal (GFP-SPP) (FIGS. 4A, 4B). Microscopy analysis revealed that expression of GFP-SPP reduces aggregation of mRFP-Q74 when compared with cells co-expressing mRFP-Q74 and control GFP (FIG. 4B). By filter trap assay, we confirmed that ectopic expression of GFP-SPP reduces the amounts of SDS-insoluble mRFP-Q74 (FIGS. 4C, 4D).

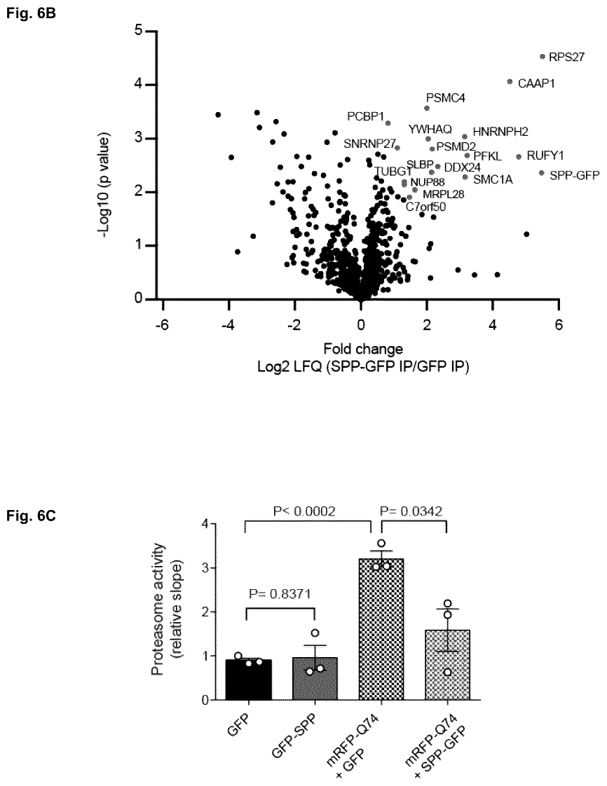

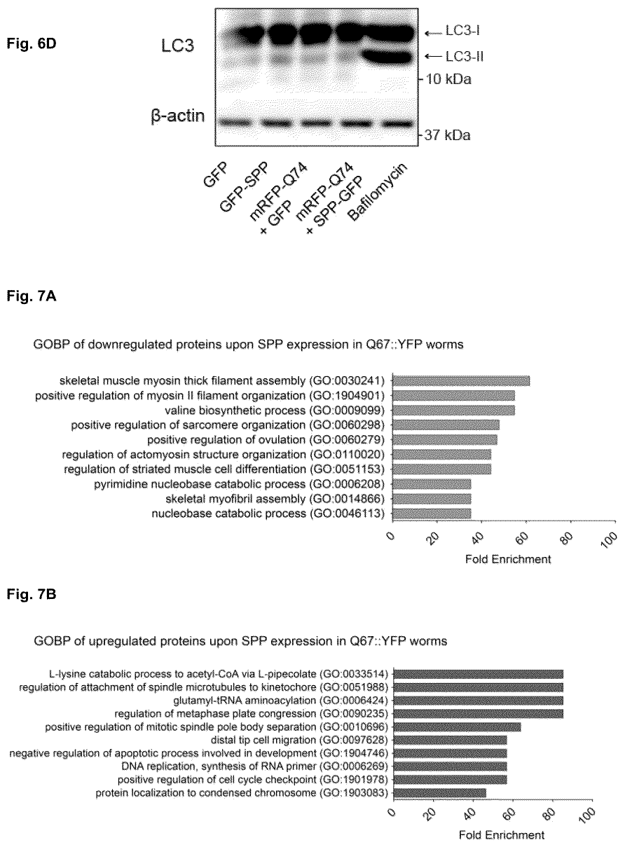

Considering the robust decline in mRFP-Q74 aggregation induced by ectopic expression of SPP, we investigated whether SPP concomitantly increases the levels of soluble mRFP-Q74. Given that insoluble/aggregated polyQ-expanded proteins do not enter the running gel, western blot assay provides a tool to quantify the levels of soluble, monomeric polyQ-proteins^{38,39}. The mRFP-Q74 protein can be detected by western blot using antibodies that recognize either the mRFP tag (anti-mCherry antibody) or the expanded polyQ stretch (anti-polyQ-expansion diseases marker)^{9,40,41}. Western blot analysis revealed two common bands of soluble mRFP-Q74 with different electrophoretic mobilities detected by both antibodies, that is a more intense band of ˜55 kDa and another band of ˜43 kDa (FIG. 6A and FIG. 4E). Notably, the levels of soluble mRFP-Q74 increased upon ectopic expression of SPP (FIGS. 4E, 4F), which correlates with the decreased amounts of aggregated mRFP-Q74 observed by filter trap assay (FIGS. 4C, 4D). While we cannot entirely rule out the possibility of SPP also cleaving mRFP-Q74 in human cells, our data primarily supports that SPP prevents the self-assembly of mRFP-Q74 into aggregates, resulting in elevated levels of the monomeric fraction (FIG. 4G).

To investigate whether SPP could affect other pathways and possibly diminish its therapeutic potential, we performed an interactome assay comparing GFP-SPP with control GFP in wild-type HEK293 cells (not shown here, published in Llamas et al 2023). We found that GFP-SPP interacts with 17 proteins of the endogenous HEK293 proteome, including 9 RNA-binding proteins involved in different processes such as splicing and translation (DDX24, HNRNPH2, RPS27, MRPL28, PCBP1, C7orf50, SLBP, SMC1A, SNRNP27) (FIG. 6B). Therefore, it is important to consider the possibility of an off-target effect of SPP on RNA metabolism. In addition to RNA-binding proteins, we found that SPP interacts with proteasome subunits (PSMD2, PSMC4) in human cells (FIG. 6B). Given that polyQ-expanded proteins also interact with proteasome subunits and can be degraded by the proteasome^{9,29} (FIGS. 2A-2C), we asked whether ectopic expression of SPP influences proteasome activity. However, we observed that SPP does not increase proteasome activity in control HEK293 cells (FIG. 6C). On the other hand, the expression of mRFP-Q74 triggered proteasome activity (FIG. 6C), which suggests a compensatory mechanism to cope with proteotoxic stress resulting from the accumulation of polyQ aggregates⁴². However, expression of SPP partially decreased the induction of proteasome activity in these cells (FIG. 6C), probably because SPP reduces polyQ74 aggregation and subsequent proteostasis collapse (FIG. 4B-4D). Although this decline in proteasome activity could contribute to the elevated levels of mRFP-Q74 detected by western blot (FIGS. 4E, 4F), it cannot explain the suppression of mRFP-Q74 aggregation induced by SPP (FIGS. 4B-4D). The autophagy-lysosome pathway can also terminate protein aggregates, but we did not observe changes in the autophagic flux upon SPP expression (FIG. 6D). Taken together, our results indicate that SPP does not activate the two major proteolytic systems.

Besides proteolytic systems, we assessed whether SPP induces conformational changes across the proteome by limited proteolysis-mass spectrometry (LiP-MS)⁴³. In the LiP-MS method, protein extracts are first subjected to protease digestion with the nonspecific proteinase K for a short time under native conditions, followed by complete digestion with the sequence-specific trypsin under denaturing conditions. This sequential protease treatment generates conformation-specific peptides, depending on the structural features of the protein, for mass spectrometry analysis⁴³. However, due to the inability of proteinase K to cleave after glutamine residues, the expanded polyQ stretch remains resistant to this protease, regardless of its conformational state⁴⁴. While LiP-MS cannot be used to distinguish changes in Q74 structure, we were able to assess thousands of other proteins (Llamas et al 2023). However, we did not find significant off-target effects on protein structure upon SPP expression after correction for multiple testing (Llamas et al 2023).

To assess the potential ameliorative effects of SPP in vivo, we used C. elegans models expressing polyQ-expanded repeats in neurons²⁷. In these animals, polyQ-expanded peptides form aggregates throughout the nervous system, with a pathogenic threshold of 40 repeats²⁷. Similar to human HEK293 cells, we found that ectopic expression of SPP reduces the amounts of neuronal Q67 aggregates while slightly increasing the levels of monomeric Q67 (FIGS. 4H-4K). The accumulation of polyQ aggregates leads to neurotoxicity and subsequent decline in the motility of the worms, resembling a disease-like phenotype^{9,10,27,41,45,46}. The severity and onset of neuronal deficits correlate with the length of the polyQ repeats²⁷. As such, polyQ67-expressing worms exhibit severe loss of motility even at early ages²⁷. Notably, ectopic expression of SPP improved the impaired motility phenotype of young Q67-expressing worms (FIG. 4L). Thus, our data indicate that SPP can prevent polyQ aggregation and subsequent neurotoxicity in C. elegans. To evaluate the effects of SPP in the context of aging, we examined C. elegans expressing polyQ40 repeats, a less aggressive polyQ stretch²⁷. We observed that ectopic expression of SPP attenuates the decline in motility of polyQ40-expressing worms during aging (FIG. 4M).

To investigate potential off-target effects of SPP expression in C. elegans, we performed quantitative proteomics analysis of polyQ67-expressing worms (Llamas et al 2023). While we were unable to quantify polyQ67 by proteomics due to lack of identifiable peptides after tryptic digestion in its sequence, we could quantify nearly 1400 other proteins. We found that SPP expression leads to a decrease in the levels of 163 proteins in Q67-expressing worms, whereas 168 proteins were upregulated (not shown here, but published in Llamas et al 2023). The downregulated proteins were enriched for factors involved in muscle myosin filament assembly, valine biosynthesis and nucleobase catabolism (FIG. 7A). On the other hand, the upregulated proteins were enriched for factors involved in L-lysine catabolism, glutamyl-tRNA aminoacylation, mitotic spindle regulation, DNA replication, and cell cycle (FIG. 7B). While some of these changes might be a consequence of the beneficial effects of SPP in preventing polyQ aggregation and neurodegeneration, we cannot rule out the possibility of off-target effects. Together, our results across different species suggest that SPP holds promise as a potential therapeutic approach for the treatment of Huntington's disease and other polyQ disorders, but potential off-target effects should be considered.

### 3. Discussion

To our knowledge, unlike mammals, plants do not experience proteinopathies caused by the abnormal aggregation of polyQ proteins. The presence of chloroplasts in plant cells potentially expands the repertoire of proteostasis components, such as chaperones and proteases, which may counteract cytosolic toxic protein aggregation. In non-plant models, the proteostasis network of subcellular compartments like the endoplasmic reticulum and nucleus can clear misfolded proteins that would otherwise be prone to aggregation when accumulated in the cytosol^{41,47,48}. Moreover, aggregated cytosolic proteins are disentangled on the mitochondria surface and subsequently imported for degradation by mitochondrial proteases⁴⁹⁻⁵¹. Considering the numerous similarities between mitochondria and chloroplast, it is plausible that parallel mechanistic pathways exist. Along these lines, we find that chloroplasts import and degrade cytosolic polyQ69-expanded protein through Clp and FtsH proteases. Conversely, impairing chloroplast import triggers the formation of Q69 aggregates in the cytosol. The unstructured configuration of Q69 protein led to the hypothesis that the polyQ region could be recognized as an unfolded N-terminal transit peptide in a pre-protein. Indeed, in-vitro import assays demonstrate that Q69 protein is imported into chloroplasts, whereas removal of the polyQ stretch hinders the import process.

We identified SPP, a protein that binds and cleaves chloroplast transit peptides, as the most enriched interactor of Q69. It has been proposed that SPP does not recognize a strict sequence motif for cleaving transit peptides, but rather recognizes transition between unfolded and folded regions of chloroplast pre-proteins^{36,37,52}. Together, our data suggest that aggregation-prone Q69 could be recognized by the chloroplast import machinery for further processing by SPP. Similarly, the human signal peptidase complex (SPC), which removes endoplasmic reticulum signal peptides, supports the degradation of misfolded proteins⁵³.

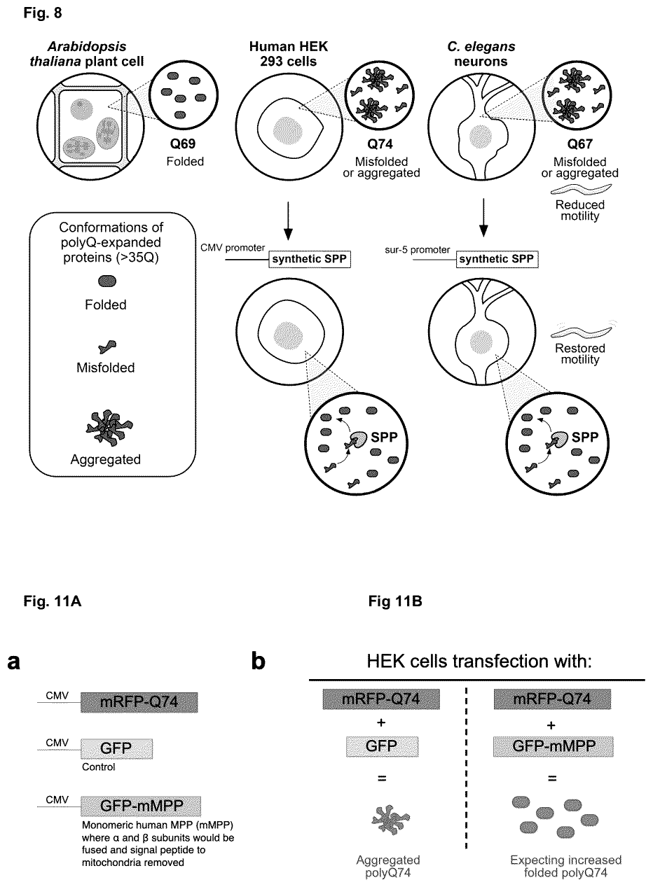

The accumulation of misfolded/aggregated proteins, leading to cell dysfunction and death, is a hallmark of age-related neurodegenerative diseases^{54,55}. Given the interaction of Q69 with SPP and the absence of aggregation in plants with functional chloroplasts, we hypothesized that plant-derived SPP could be a potential treatment for human polyQ-related neurodegenerative diseases. In recent years, there has been increasing interest in using plant proteins as therapeutic agents for human diseases. For instance, nanothylakoids containing photosynthetic proteins have been introduced into animal cells to restore anabolism in certain diseases and supply cells with ATP and NADPH⁵⁶. Moreover, ectopic expression of plant RDR1 can inhibit cancer cell proliferation⁵⁷. In the present application, we show that SPP can be expressed in human cells and worm models to prevent polyQ aggregation (FIG. 8). Beyond SPP, our interactome data of polyQ-expanded proteins in plants provide a plethora of potential therapeutic targets that can be explored in future studies.

While our findings raise the intriguing prospect of utilizing SPP and other SPP-like proteins as therapeutic agents

### Mouse Model

Mangiarini et al 1996 developed a mice model that are transgenic for the 5′ end of the human protein HD (HTT) gene carrying (CAG)-(CAG)150 repeat expansions. The advantage of the transgenic mice is that the mice exhibits many of the features of HD, including choreiform-like movements, involuntary stereotypic movements, tremor, and epileptic seizures, as well as nonmovement disorder components. Presently, the mice model is used to show the effect of the protein according to the present invention in order to assess the effects on the molecular pathology of HD.

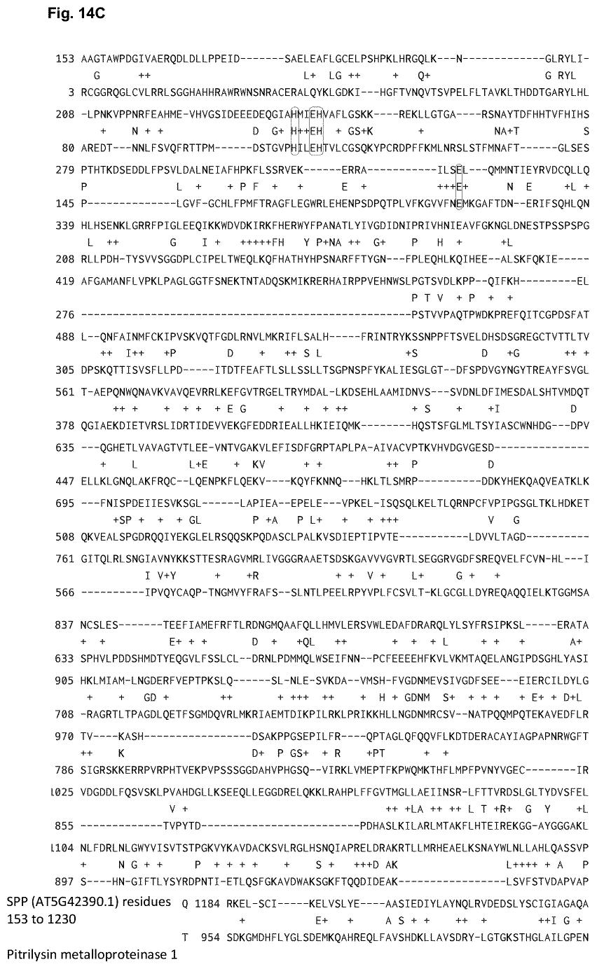

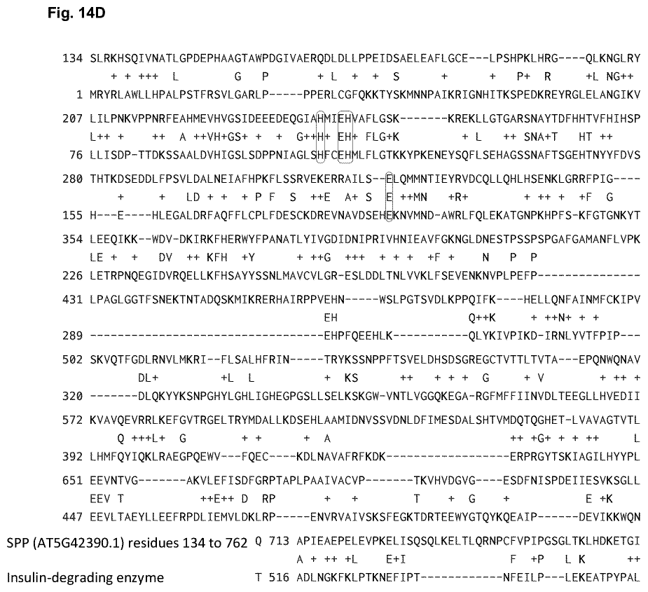

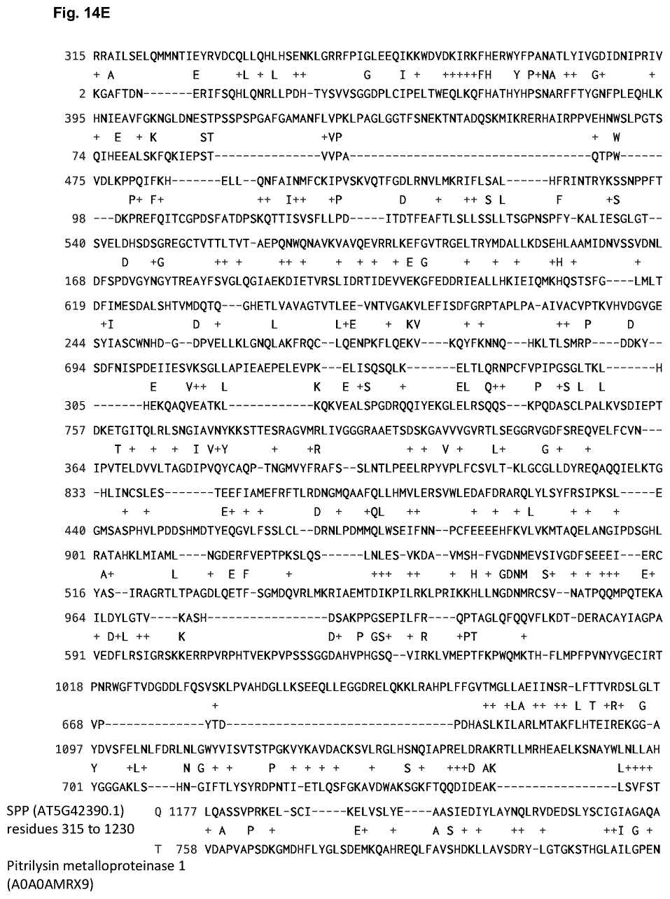

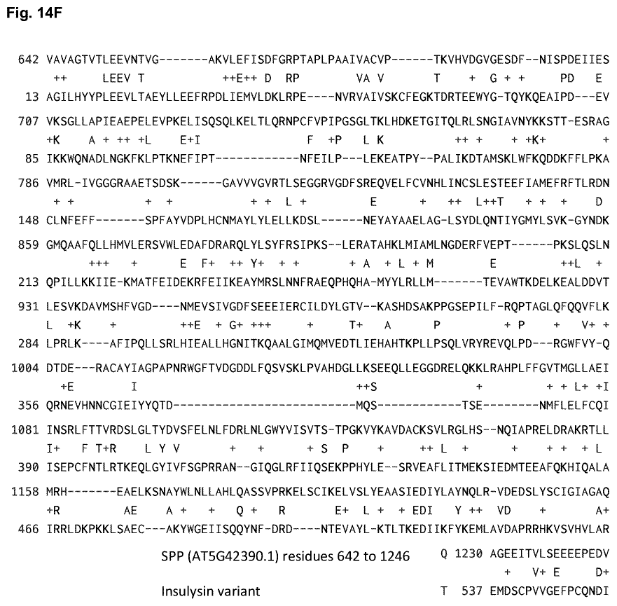

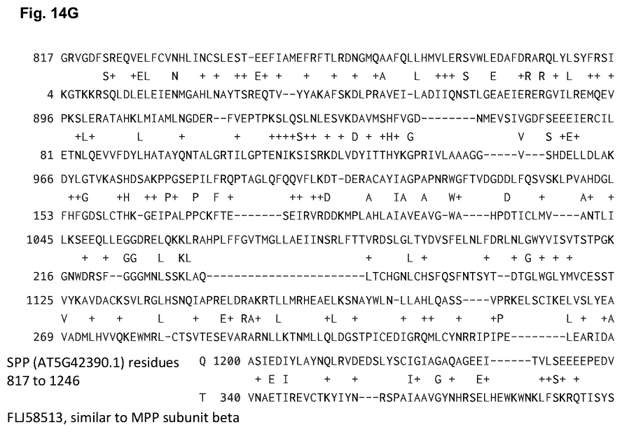

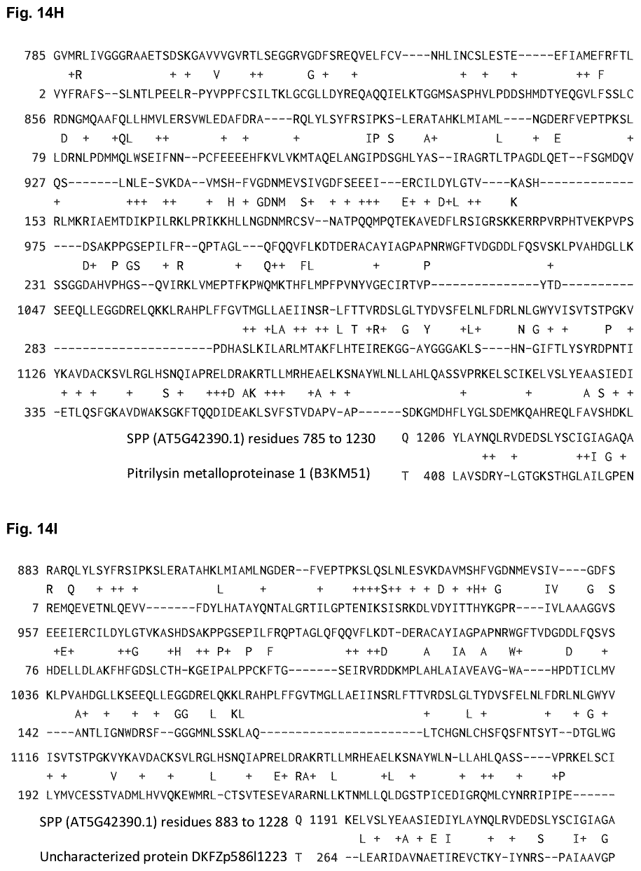

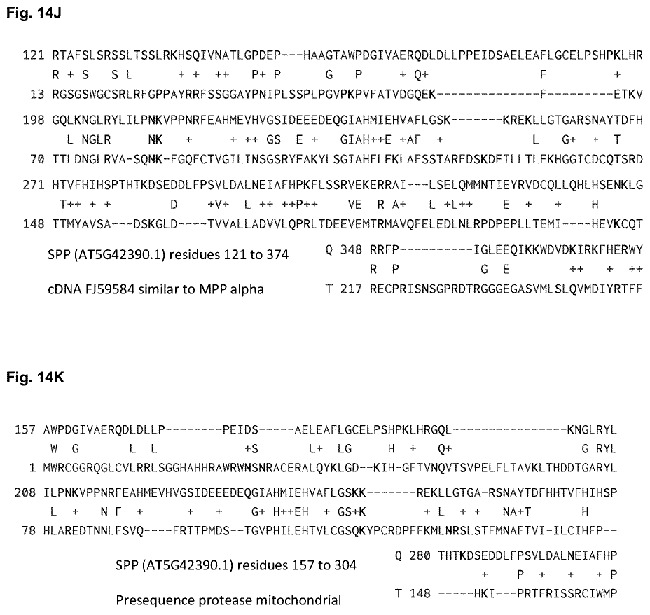

A Blast search using the complete amino acid sequence of SPP isoform 1 (Seq ID No. 20) of Arabidopsis thaliana identified 55 top Blast hits with significant homology to human proteins (not shown). Among the 55 Blast hits the isoform p of the mitochondrial-processing peptidase (MPP, aa sequence shown in Seq ID No. 26, encoded by Seq ID No. 7) (31.69% identity), Nardilysin (27.09% identity), and the Insulin-Degrading Enzyme (IDE) (24.32% identity) have been identified. Complementary to the protein Blast analysis, we used Foldseek to detect distant evolutionary relationships between the Arabidopsis SPP and human proteins based on predicted 3D structures. The Foldseek analysis, indicates that SPP protein has 14 homologs including the two subunits, α and β, of the human MPP (Shown in Table 5), Nardilysin (shown in Seq ID No. 33 or in Seq ID No. 34, encoded by Seq ID No. 14 or Seq ID No. 15), the IDE (shown in Seq ID No. 36, encoded by Seq ID No. 17 or Seq ID No. 18) and Pitrilysin metalloproteinase 1 (shown in Seq ID No. 35, encoded by Seq ID No. 16) among other proteins (Table 6 and FIGS. 14A-14K).

TABLE 6
Foldseek Alignment Results. This table presents the top hits from a Foldseek alignment
of our query protein SPP against a Homo sapiens protein structure database.

| | | | | | Target |
|---|---|---|---|---|---|
| Target | Description | E-Value | Score | Query pos. | Pos. |
| AF-Q96L67-F1- | Nardllysln | 1.85e−23 | 637 | 95- | 31-901 |
| model v4 | | | | 994(1265) | (948) |
| AF-B4DYVO-F1- | cDNA FLJ59785 | 3.98e−20 | 587 | 125- | 1-740 |
| model v4 | highly similar to SPP | | | 962(1265) | (747) |
| AF-A0A7I2YQT2- | Pitrilysin | 8.83e−23 | 585 | 153- | 3-1009 |
| F1-model v4 | metalloproteinase 1 | | | 1230(1265) | (1021) |
| AF-075439-F1- | Mitochondrial-processing | 5.64e−16 | 538 | 122- | 1-484 |
| model v4 | peptidase | | | 707(1265) | (489) |
| AF-A0A7I2V612- | Insulin-degrading enzyme | 7.78e−16 | 437 | 134- | 1-550 |
| F1-model v4 | | | | 762(1265) | (609) |
| AF-A0A0A0MRX9- | Pitrilysin | 7.06e−15 | 408 | 315- | 2-820 |
| F1-model v4 | metalloproteinase 1 | | | 1230(1265) | (832) |
| AF-A0A7I2V634- | Insulin-degrading enzyme | 1.45e−11 | 384 | 142- | 1-383 |
| F1-modal v4 | | | | 566(1265) | (384) |
| AF-059GA5-F1- | Insulysin variant | 2.89e−11 | 309 | 642-1246 | 13-553 |
| model V4 | | | | (1265) | (594) |
| AF-B4DM90-F1- | cDNA FLJ58513 | 4.72e−8 | 271 | 817- | 4-389 |
| model v4 | highly similar to SPP | | | 1246(1265) | (403) |
| AF-B3KM51-F1- | Pitrilysin metalloproteinase | 1.13e−7 | 233 | 785- | 2-431 |
| model v4 | 1 | | | 1230(1265) | (443) |
| AF-09UG64-F1- | Uncharacterized protein | 2.67e−5 | 183 | 883- | 7-297 |
| model V4 | DKFZp58 | | | 1228(1265) | (316) |
| AF-B4DRK5-F1- | cDNA FLJ59584 | 2.21e−4 | 178 | 121- | 13- |
| model V4 | highly similar to SPP | | | 374(1265) | 253(257) |
| AF-05SXN9-F1- | Alpha-MPP | 1.27e−3 | 148 | 1004- | 21- |
| model v4 | | | | 1263(1265) | 250(271) |
| AF-A0A7I2V5K2- | Presequence protease, | 5.44e+0 | 36 | 157- | 1-165 |
| F1-model V4 | mitochondrial | | | 304(1265) | (204) |

The two subunits of the human mitochondrial processing peptidase (MPP) are similar to the monomeric SPP of Arabidopsis thaliana. From our Foldseek search against human proteins that might be similar to SPP, we found a structural alignment (FIGS. 13A-13B). between the SPP and the β subunit of the human MPP (also named PMPCB) of which the aa sequence shown in Seq. ID No. 26, encoded by Seq ID No. 7 Importantly The Zn²⁺-binding region that corresponds to the conserved HxxEHx₇₆E amino acid motif is conserved in the SPP (Seq. ID No. 31 encoded by nucleic acid sequence Seq ID No. 12) and the in the MPP β subunit (Seq. ID No. 32 encoded by nucleic acid sequence Seq ID No. 13). An alignment of aa sequences is shown below:

Seq ID No. 31
HMIEHVAFLG SKKREKLLGT GARSNAYTDF HHTVFHIHSP
Seq ID No. 32
HFLEHMAFKG TKKRSQLDLE LEIENMGAHL NAYTSREQTV
Seq ID No. 31
THTKDSEDDL FPSVLDALNE IAFHPKFLSS RVEKERRAIL SE
Seq ID No. 32
YYAKAFSKDL PRAVEILADI IQNSTLGEAE IERERGVILR E

Moreover, SPP structure aligns from residues 1004 to 1263 of SPP (shown in Seq ID No. 30 encoded by Seq ID No. 11) with the a subunit of the MPP (Also known as PMPCA) of which the amino acid sequence is shown in Seq ID No. 27, encoded by Seq ID No. 8. Remarkably, the conserve glycine-rich loop “GGGGSFSAGGPGKGMFS” shown in Seq ID No. 28 encoded by Seq ID No. 9) which is essential for substrate biding (Nagao et al., 2000, Dvorakova-Hola et al., 2010) and which moves the precursor protein towards the active site through a multistep process (Kucera et al., 2013) is missing in the plant SPP protein (FIG. 12A).

While the β subunit of the MPP aligns at the N-ter (aa residues 122-707 of SPP are shown in Seq ID No. 29 encoded by Seq ID No. 10) the alignment with the a subunit of the MPP aligns downstream close to the C-ter (residues 1004-1253) (FIGS. 13A and 12A). In most eukaryotes, the active MPP consists of the α and β subunits, which bind together to form a heterodimeric complex (Taylor et al., 2001). The subunits of the MPP are highly conserved, and have shown to be interoperable between species (Adamec et al., 1999). These observations suggests that plant SPP, may function similar to MPP but the complete active enzyme is codified for one gene. In fact, the principal responsibility of the MPP and the SPP is to remove the N-terminal targeting pre-sequences of protein imported into mitochondria or chloroplasts (Trosch & Jarvis, 2011, Kunova et al., 2022).

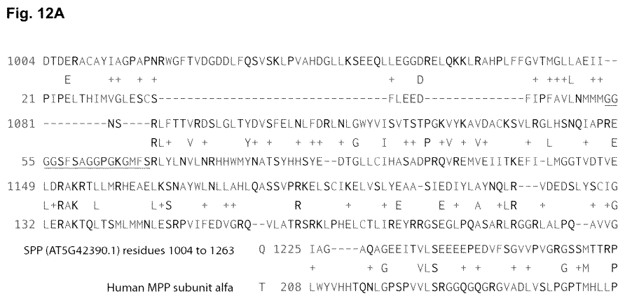

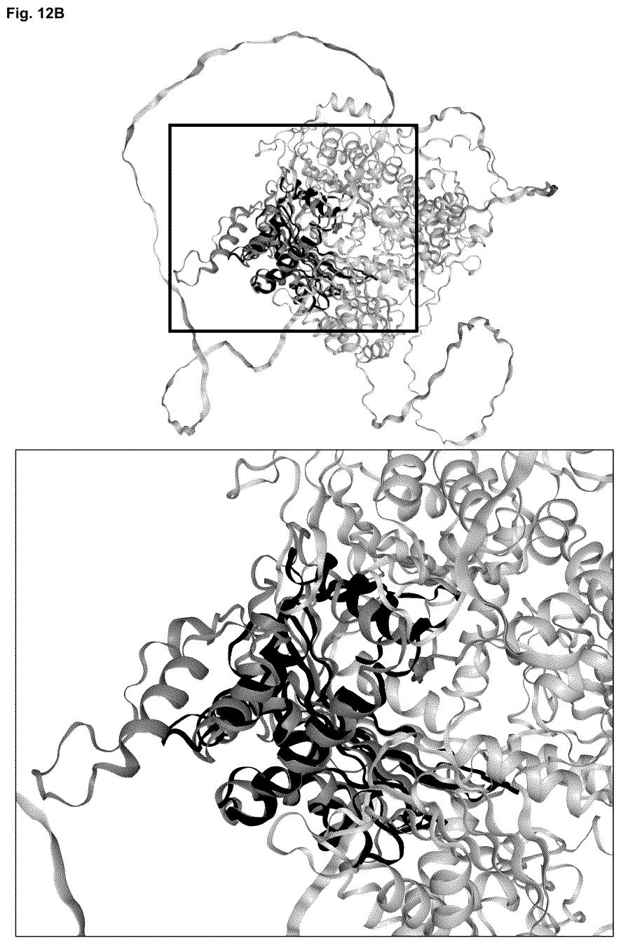

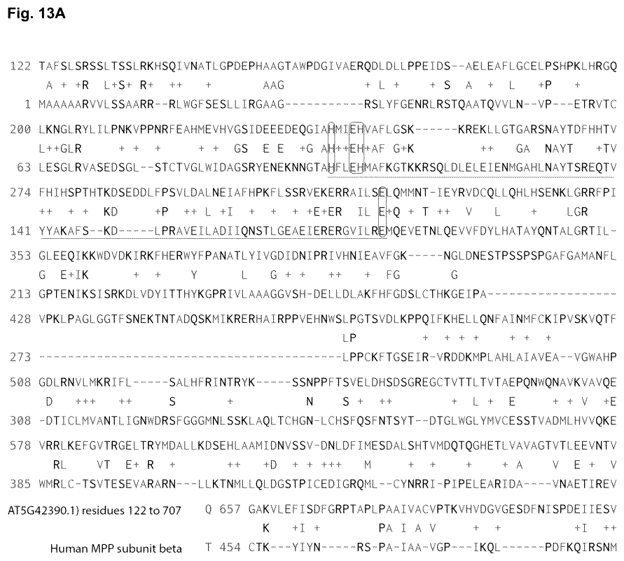

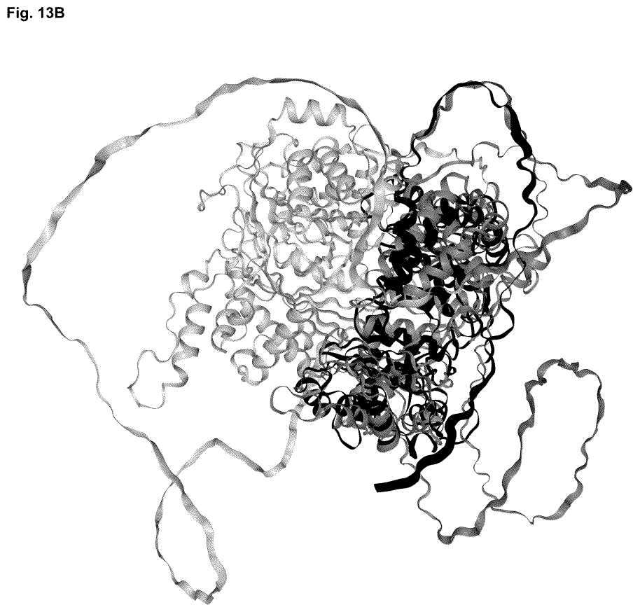

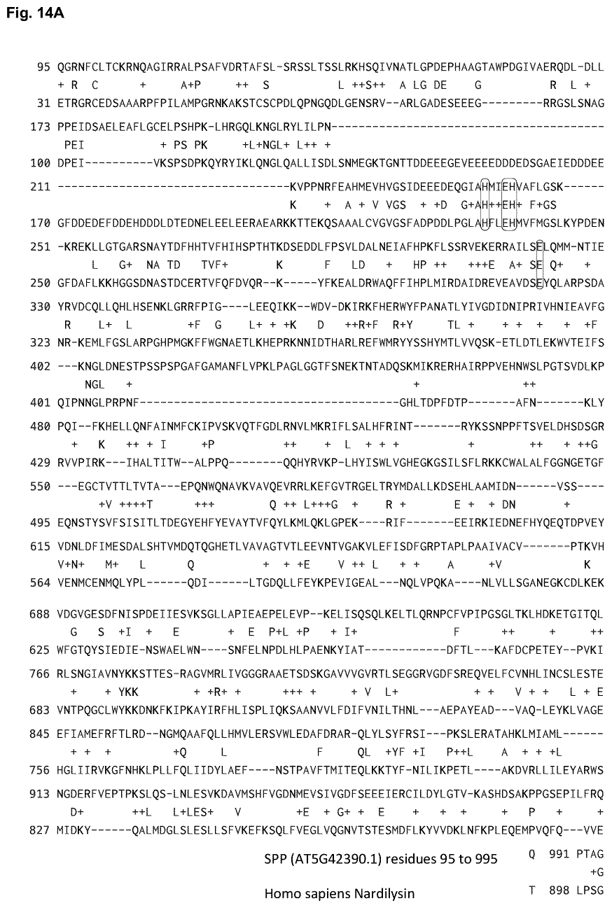

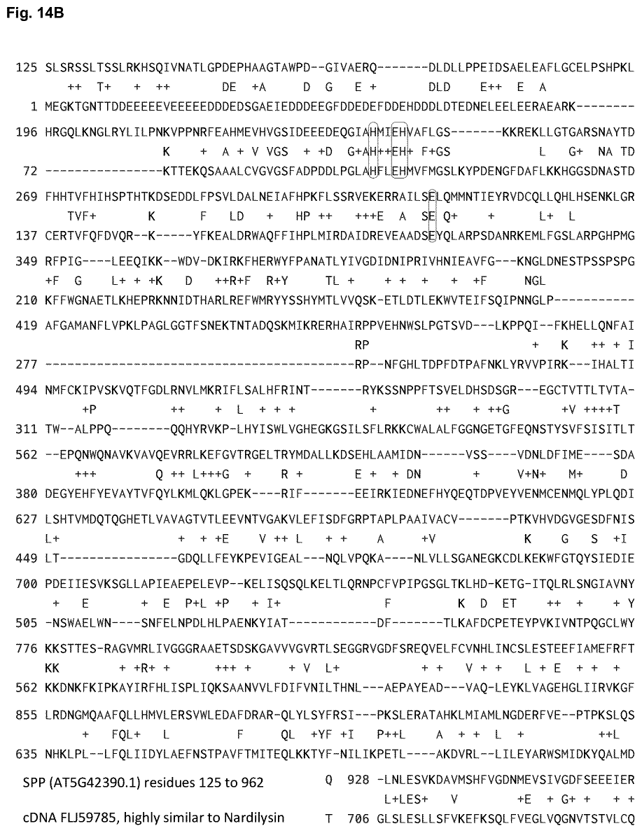

In order to test if the human MPP and other modified peptidases are also able to reduce aggregation, or cleave polyQ proteins, we perform transient expression assays in human HEK cells expressing polyQ-extended proteins. We will access the ability of human peptidases to reduce polyQ aggregation based on our previous assays methods described herein and shown in FIGS. 4A-4G. Similar to SPP, a monomeric human MPP (encoded by one single gene and without a mitochondrial targeting peptide) is generated where subunits α and β are connected by linkers based on the structural feature of the monomeric SPP (FIG. 11A). We will test the role of the conserved Zn²⁺-binding motif and the glycine-rich binding loop in clearing polyQ aggregates (FIG. 11B).

Analyzing the anti-aggregation activities of other peptidases One of the major challenges in delivering drugs to the brain is to overcome the blood-brain barrier (BBB), which restricts the passage of most molecules from the blood to the central nervous system.

Having a small active peptidase, could have enhanced permeability to cross the BBB. Using the HEK cells transient expression explained above (FIGS. 11A-11B), we will evaluate the anti-aggregation activities of smaller SPP-like proteins or peptidases similar to SPP, such as the Nardisilyn and IDE.

The IDE shows structural similarities to the N-ter of SPP (142-566) and maintains the Zn²⁺-binding motif (HxxEHx₇₆E) responsible for the peptidase activity (FIG. 14E). Interestingly, IDE can degrade amyloid beta (Aβ), a peptide implicated in the pathogenesis of Alzheimer's disease (Kurochkin & Goto, 1994). Experiments in HEK cells as proposed in FIG. 11B will be performed with the IDE enzyme, and other peptidases, to assess whether human proteins can degrade or reduce aggregation of proteins containing extended polyQ regions.

### REFERENCES

1 Hommen, F., Bilican, S. & Vilchez, D. Protein clearance strategies for disease intervention. J Neural Transm (Vienna), (2021).
2 Ross, E. D., Baxa, U. & Wickner, R. B. Scrambled prion domains form prions and amyloid. Mol Cell Biol 24, 7206-7213, (2004).
3 Shorter, J. & Lindquist, S. Prions as adaptive conduits of memory and inheritance. Nat Rev Genet 6, 435-450, (2005).
4 Nath, S. R. & Lieberman, A. P. The Ubiquitination, Disaggregation and Proteasomal Degradation Machineries in Polyglutamine Disease. Front Mol Neurosci 10, 78, (2017).
5 Kitamura, A. et al. Cytosolic chaperonin prevents polyglutamine toxicity with altering the aggregation state. Nat Cell Biol 8, 1163-1170, (2006).
6 Gruber, A. et al. Molecular and structural architecture of polyQ aggregates in yeast. Proc Natl Acad Sci USA 115, E3446-E3453, (2018).
7 Doi, H. et al. Identification of ubiquitin-interacting proteins in purified polyglutamine aggregates. FEBS Lett 571, 171-176, (2004).
8 Braun, R. J., Buttner, S., Ring, J., Kroemer, G. & Madeo, F. Nervous yeast: modeling neurotoxic cell death. Trends Biochem Sci 35, 135-144, (2010).
9 Koyuncu, S. et al. The ubiquitin ligase UBR5 suppresses proteostasis collapse in pluripotent stem cells from Huntington's disease patients. Nat Commun 9, 2886, (2018).
10 Noormohammadi, A. et al. Somatic increase of CCT8 mimics proteostasis of human pluripotent stem cells and extends C. elegans lifespan. Nat Commun 7, 13649, (2016).
11 Finkbeiner, S. Huntington's Disease. Cold Spring Harb Perspect Biol 3, (2011).
12 Koyuncu, S., Fatima, A., Gutierrez-Garcia, R. & Vilchez, D. Proteostasis of Huntingtin in Health and Disease. Int J Mol Sci 18, (2017).
13 Saudou, F. & Humbert, S. The Biology of Huntingtin. Neuron 89, 910-926, (2016).
14 Cattaneo, E., Zuccato, C. & Tartari, M. Normal huntingtin function: an alternative approach to Huntington's disease. Nat Rev Neurosci 6, 919-930, (2005).
15 Pearce, M. M. P. & Kopito, R. R. Prion-Like Characteristics of Polyglutamine-Containing Proteins. Cold Spring Harb Perspect Med 8, (2018).
16 Yang, J. & Yang, X. Phase Transition of Huntingtin: Factors and Pathological Relevance. Front Genet 11, 754, (2020).
17 Peskett, T. R. et al. A Liquid to Solid Phase Transition Underlying Pathological Huntingtin Exon1 Aggregation. Mol Cell 70, 588-601 e586, (2018).
18 Ranum, L. P. et al. Spinocerebellar ataxia type 1 and Machado-Joseph disease: incidence of CAG expansions among adult-onset ataxia patients from 311 families with dominant, recessive, or sporadic ataxia. Am J Hum Genet 57, 603-608, (1995).
19 Kawaguchi, Y. et al. CAG expansions in a novel gene for Machado-Joseph disease at chromosome 14q32.1. Nat Genet 8, 221-228, (1994).
20 Kottenhagen, N., Gramzow, L., Horn, F., Pohl, M. & Theissen, G. in German Conference on Bioinformatics 2012 Vol. 26 (eds S. Bocker et al.) (2012).
21 Jung, J. H. et al. A prion-like domain in ELF3 functions as a thermosensor in Arabidopsis. Nature 585, 256-260, (2020).
22 Dorone, Y. et al. A prion-like protein regulator of seed germination undergoes hydration-dependent phase separation. Cell 184, 4284-4298 e4227, (2021).
23 Chakrabortee, S. et al. Luminidependens (LD) is an Arabidopsis protein with prion behavior. Proc Natl Acad Sci USA 113, 6065-6070, (2016).
24 Alberti, S. The plant response to heat requires phase separation. Nature 585, 191-192, (2020). Mangiarini, L. et al. Exon 1 of the HD gene with an expanded CAG repeat is sufficient to cause a progressive neurological phenotype in transgenic mice. Cell 87, 493-506, (1996).
26 Morley, J. F., Brignull, H. R., Weyers, J. J. & Morimoto, R. I. The threshold for polyglutamine-expansion protein aggregation and cellular toxicity is dynamic and influenced by aging in Caenorhabditis elegans. Proc Natl Acad Sci USA 99, 10417-10422, (2002).
27 Brignull, H. R., Moore, F. E., Tang, S. J. & Morimoto, R. I. Polyglutamine proteins at the pathogenic threshold display neuron-specific aggregation in a pan-neuronal Caenorhabditis elegans model. J Neurosci 26, 7597-7606, (2006).
28 Wang, H. et al. Suppression of polyglutamine-induced toxicity in cell and animal models of Huntington's disease by ubiquilin. Hum Mol Genet 15, 1025-1041, (2006).
29 Kim, Y. E. et al. Soluble Oligomers of PolyQ-Expanded Huntingtin Target a Multiplicity of Key Cellular Factors. Mol Cell 63, 951-964, (2016).
30 Lee, D. W. & Hwang, I. Understanding the evolution of endosymbiotic organelles based on the targeting sequences of organellar proteins. New Phytol 230, 924-930, (2021).
31 Lee, D. W., Jung, C. & Hwang, I. Cytosolic events involved in chloroplast protein targeting. Biochim Biophys Acta 1833, 245-252, (2013).
32 Sun, J. L., Li, J. Y., Wang, M. J., Song, Z. T. & Liu, J. X. Protein Quality Control in Plant Organelles: Current Progress and Future Perspectives. Mol Plant 14, 95-114, (2021).
33 Thomson, S. M., Pulido, P. & Jarvis, R. P. Protein import into chloroplasts and its regulation by the ubiquitin-proteasome system. Biochem Soc Trans 48, 71-82, (2020).
34 Llamas, E., Pulido, P. & Rodriguez-Concepcion, M. Interference with plastome gene expression and Clp protease activity in Arabidopsis triggers a chloroplast unfolded protein response to restore protein homeostasis. PLoS Genet 13, e1007022, (2017).
35 Wu, G. Z. et al. Control of retrograde signalling by protein import and cytosolic folding stress. Nat Plants 5, 525-538, (2019).
36 Zhong, R., Wan, J., Jin, R. & Lamppa, G. A pea antisense gene for the chloroplast stromal processing peptidase yields seedling lethals in Arabidopsis: survivors show defective GFP import in vivo. Plant J 34, 802-812, (2003).
37 Rowland, E. et al. The CLP and PREP protease systems coordinate maturation and degradation of the chloroplast proteome in Arabidopsis thaliana. New Phytol, (2022).
38 Juenemann, K., Wiemhoefer, A. & Reits, E. A. Detection of ubiquitinated huntingtin species in intracellular aggregates. Front Mol Neurosci 8, 1, (2015).
39 Miller, V. M. et al. CHIP suppresses polyglutamine aggregation and toxicity in vitro and in vivo. J Neurosci 25, 9152-9161, (2005).
40 Balaji, V. et al. A dimer-monomer switch controls CHIP-dependent substrate ubiquitylation and processing. Mol Cell 82, 3239-3254 e3211, (2022).
41 Lee, H. J. et al. Cold temperature extends longevity and prevents disease-related protein aggregation through PA28γ-induced proteasomes. Nature Aging 3, 546-566, (2023).
42 Vilchez, D., Saez, I. & Dillin, A. The role of protein clearance mechanisms in organismal ageing and age-related diseases. Nat Commun 5, 5659, (2014).
43 Schopper, S. et al. Measuring protein structural changes on a proteome-wide scale using limited proteolysis-coupled mass spectrometry. Nat Protoc 12, 2391-2410, (2017).
44 Juenemann, K. et al. Expanded polyglutamine-containing N-terminal huntingtin fragments are entirely degraded by mammalian proteasomes. J Biol Chem 288, 27068-27084, (2013).
45 Calculli, G. et al. Systemic regulation of mitochondria by germline proteostasis prevents protein aggregation in the soma of C. elegans. Sci Adv 7, (2021).
46 Gidalevitz, T., Ben-Zvi, A., Ho, K. H., Brignull, H. R. & Morimoto, R. I. Progressive disruption of cellular protein folding in models of polyglutamine diseases. Science 311, 1471-1474, (2006).
47 Liu, F., Koepp, D. M. & Walters, K. J. Artificial targeting of misfolded cytosolic proteins to endoplasmic reticulum as a mechanism for clearance. Sci Rep-Uk 5, (2015).
48 Mediani, L. et al. Defective ribosomal products challenge nuclear function by impairing nuclear condensate dynamics and immobilizing ubiquitin. EMBO J 38, e101341, (2019).
49 Ruan, L. et al. Cytosolic proteostasis through importing of misfolded proteins into mitochondria. Nature 543, 443-446, (2017).
50 Li, Y. et al. A mitochondrial FUNDC1/HSC70 interaction organizes the proteostatic stress response at the risk of cell morbidity. EMBO J 38, (2019).
51 Schlagowski, A. M. et al. Increased levels of mitochondrial import factor Mia40 prevent the aggregation of polyQ proteins in the cytosol. EMBO J 40, e107913, (2021).
52 Gavel, Y. & von Heijne, G. A conserved cleavage-site motif in chloroplast transit peptides. FEBS Lett 261, 455-458, (1990).
53 Zanotti, A. et al. The human signal peptidase complex acts as a quality control enzyme for membrane proteins. Science 378, 996-1000, (2022).
54 Soto, C. & Pritzkow, S. Protein misfolding, aggregation, and conformational strains in neurodegenerative diseases. Nat Neurosci 21, 1332-1340, (2018).
55 Lopez-Otin, C., Blasco, M. A., Partridge, L., Serrano, M. & Kroemer, G. Hallmarks of aging: An expanding universe. Cell 186, 243-278, (2023).
56 Chen, P. et al. A plant-derived natural photosynthetic system for improving cell anabolism. Nature 612, 546-554, (2022).
57 Qi, Y. et al. A plant immune protein enables broad antitumor response by rescuing microRNA deficiency. Cell 185, 1888-1904 e1824, (2022).
58 Woodson, J. D. et al. Ubiquitin facilitates a quality-control pathway that removes damaged chloroplasts. Science 350, 450-454, (2015).
59 Ling, Q. et al. Ubiquitin-dependent chloroplast-associated protein degradation in plants. Science 363, (2019).
60 Llamas, E. et al. The intrinsic chaperone network of Arabidopsis stem cells confers protection against proteotoxic stress. Aging Cell 20, e13446, (2021).
61 Narain, Y., Wyttenbach, A., Rankin, J., Furlong, R. A. & Rubinsztein, D. C. A molecular investigation of true dominance in Huntington's disease. J Med Genet 36, 739-746, (1999).
62 Clough, S. J. & Bent, A. F. Floral dip: a simplified method for Agrobacterium-mediated transformation of Arabidopsis thaliana. Plant J 16, 735-743, (1998).
63 Lancaster, A. K., Nutter-Upham, A., Lindquist, S. & King, O. D. PLAAC: a web and command-line application to identify proteins with prion-like amino acid composition. Bioinformatics 30, 2501-2502, (2014).
64 Erdos, G., Pajkos, M. & Dosztanyi, Z. IUPred3: prediction of protein disorder enhanced with unambiguous experimental annotation and visualization of evolutionary conservation. Nucleic Acids Res 49, W297-W303, (2021).
65 Ling, Q. H. & Jarvis, P. Analysis of Protein Import into Chloroplasts Isolated from Stressed Plants. Jove-J Vis Exp, (2016).
66 Brenner, S. The genetics of Caenorhabditis elegans. Genetics 77, 71-94, (1974).
67 Mello, C. C., Kramer, J. M., Stinchcomb, D. & Ambros, V. Efficient gene transfer in C. elegans: extrachromosomal maintenance and integration of transforming sequences. EMBO J 10, 3959-3970, (1991).
68 Tyanova, S., Temu, T. & Cox, J. The MaxQuant computational platform for mass spectrometry-based shotgun proteomics. Nat Protoc 11, 2301-2319, (2016).
69 Tyanova, S. et al. The Perseus computational platform for comprehensive analysis of (prote)omics data. Nat Methods 13, 731-740, (2016).
70 Demichev, V., Messner, C. B., Vernardis, S. I., Lilley, K. S. & Ralser, M. DIA-NN: neural networks and interference correction enable deep proteome coverage in high throughput. Nat Methods 17, 41-44, (2020).
71 Llamas, E. et al. The intrinsic chaperone network of Arabidopsis stem cells confers protection against proteotoxic stress. Aging Cell 20, (2021).
72 Adamec, J., Gakh, O., Spizek, J. and Kalousek, F. (1999) Complementation between mitochondrial processing peptidase (MPP) subunits from different species. Arch Biochem Biophys, 370, 77-85.
73 Dvorakova-Hola, K., Matuskova, A., Kubala, M., Otyepka, M., Kucera, T., Vecer, J., et al. (2010) Glycine-rich loop of mitochondrial processing peptidase alpha-subunit is responsible for substrate recognition by a mechanism analogous to mitochondrial receptor Tom20. J Mol Biol, 396, 1197-1210.
74 Kucera, T., Otyepka, M., Matuskova, A., Samad, A., Kutejova, E. and Janata, J. (2013) A computational study of the glycine-rich loop of mitochondrial processing peptidase. PLoS One, 8, e74518.
75 Kunova, N., Havalova, H., Ondrovicova, G., Stojkovicova, B., Bauer, J. A., Bauerova-Hlinkova, V., et al. (2022) Mitochondrial Processing Peptidases-Structure, Function and the Role in Human Diseases. Int J Mol Sci, 23.
76 Kurochkin, I. V. and Goto, S. (1994) Alzheimer's beta-amyloid peptide specifically interacts with and is degraded by insulin degrading enzyme. FEBS Lett, 345, 33-37.
77 Llamas, E., Koyuncu, S., Lee, H. J., Wehrmann, M., Gutierrez-Garcia, R., Dunken, N., et al. (2023) In planta expression of human polyQ-expanded huntingtin fragment reveals mechanisms to prevent disease-related protein aggregation. Nat Aging, 3, 1345-1357.
78 Nagao, Y., Kitada, S., Kojima, K., Toh, H., Kuhara, S., Ogishima, T. and Ito, A. (2000) Glycine-rich region of mitochondrial processing peptidase alpha-subunit is essential for binding and cleavage of the precursor proteins. J Biol Chem, 275, 34552-34556.
79 Taylor, A. B., Smith, B. S., Kitada, S., Kojima, K., Miyaura, H., Otwinowski, Z., et al. (2001) Crystal structures of mitochondrial processing peptidase reveal the mode for specific cleavage of import signal sequences. Structure, 9, 615-625.
80 Trosch, R. and Jarvis, P. (2011) The stromal processing peptidase of chloroplasts is essential in Arabidopsis, with knockout mutations causing embryo arrest after the 16-cell stage. PLoS One, 6, e23039.
81 Hueng-Chuen Fan, Li-Ing Ho, Ching-Shiang Chi, Shyi-Jou Chen, Giia-Sheun Peng, Tzu-Min Chan, Shinn-Zong Lin, and Horng-Jyh Harn, M. D., Ph.D. Polyglutamine (PolyQ) Diseases: Genetics to Treatments Cell Transplantation Volume 23, Issue 4-5, May 2014, Pages 441-458.
82 Diana Kwon, Nature 593, 180 (2021): Failure of genetic therapies for Huntington's devastates community https://doi.org/10.1038/d41586-021-01177-7.

1. A protein exhibiting an antiaggregating activity and/or disaggregating activity toward a target protein comprising an extended polyQ stretch and comprising a Zn²⁺-binding region, wherein its amino acid (aa) sequence comprises a conserved motif HxxEHx₇₅₋₈₀E, wherein x is any amino acid, and wherein the enzyme comprises at least one conservative amino acid substitution or a deletion in at least one or more positions x compared to the corresponding amino acid in the naturally occurring enzyme from which the motif is derived.

2. (canceled)

3. The protein of claim 1, wherein the Zn²⁺-binding region supports or enables an interaction with at least one carbonyl group of the target protein.

4. The protein of claim 1, wherein a variant of the protein, comprising at least one mutation in at least one position of H or E at any non-x location in the conserved motif, exhibits a decreased antiaggregating activity and/or disaggregating activity or full loss of antiaggregating activity and/or disaggregating activity.

5. The protein of claim 1, that lacks a functional N-terminal signal peptide for translocation through a lipid bilayer for import into mitochondria or chloroplasts.

6. The protein of claim 1, wherein the protein further comprises a glycine-rich loop of at least 5, 6, 7, or 8 glycine residues.

7. The protein of claim 6, wherein the glycine-rich loop has the amino acid sequence GGGGSFSAGGPGKGMFS (Seq ID No. 28) or any amino acid sequence, wherein the amino acids other than G are arbitrarily exchangeable.

8.-11. (canceled)

12. The protein of claim 1, wherein the protein comprises one or more subunits, at least one or more fragments of any subunit of the protein, at least one or more fragments of the protein, or it is a derivative of any combination of the aforementioned components.

13. The protein of claim 1, wherein the protein is a a genetically modified stromal processing peptidase (SPP) a human derived and genetically modified SPP-like protein, a fusion protein, or a hybrid protein.

14. (canceled)

15. (canceled)

16. The protein of claim 1, wherein the protein is a plant derived stromal processing peptidase (SPP) from an Arabidopsis species, a mitochondrial-processing peptidase (MPP) or any derivative thereof, Nardilysin or any derivative thereof, an Insulin-Degrading Enzyme (IDE) or any derivative thereof, or any combination of the aforementioned.

17. A nucleic acid construct encoding the protein of claim 1, wherein the nucleic acid construct comprises a sequence encoding a Zn²⁺-binding region comprising the conserved motif HxxEHx₇₅₋₈₀E.

18. The nucleic acid construct of claim 17, which does not encode a functional N-terminal signal peptide or which does not encode an N-terminal signal peptide at all.

19. The nucleic acid construct of claim 17, wherein the nucleotide sequence encoding the protein is optionally codon-optimized and wherein the nucleic acid construct optionally comprises a sequence encoding a heterologous promoter.

20. (canceled)

21. A method of inhibiting or preventing aggregation of an aggregation-prone polyQ protein or a fragment thereof, which method comprises allowing a protein comprising a Zn²⁺-binding region exhibiting antiaggregating activity and/or disaggregating activity to conic into contact with the aggregation-prone polyQ protein or fragment thereof.

22. A method of disaggregating aggregates of an aggregation-prone polyQ protein or a fragment thereof, which method comprises allowing a protein comprising a Zn-binding region and exhibiting a disaggregating activity to come into contact with the aggregates.

23. The method of claim 21 in which the aggregation-prone polyQ protein or fragment thereof comprises a stretch of greater than 18 glutamine residues.

24. The method of claim 23, wherein the stretch of glutamine residues is contiguous.

25. The method of claim 21, in which the protein comprises a plant derived stromal processing peptidase (SPP) from an Arabidopsis species, preferably from Arabidopsis thaliana, a mitochondrial-processing peptidase (MNPP), Nardilysin, an Insulin-Degrading Enzyme (IDE) or any combination of the aforementioned proteins or of any fragments of the aforementioned proteins.

26. The method of claim 21, which is an in vitro method, an ex vivo method, or an in vivo method.

27. The method of claim 21, which comprises expressing the protein in a eukaryotic cell under conditions in which the aggregation-prone polyQ protein or fragment thereof is disaggregated, cleaved, or both disaggregated and cleaved.

28. The method claim 21, wherein the aggregation-prone polyQ protein or a fragment thereof is: (i) a Huntingtin (HTT) protein or a variant thereof comprising an expanded polyQ sequence compared to wild type HTT protein, or (ii) Ataxin3 or a variant thereof comprising an expanded polyQ sequence compared to wild type Ataxin3.

29.-32. (canceled)


SEQUENCE LISTING (55 sequences)

SEQ ID NO: 1 - DNA, 3798 bp, Arabidopsis thaliana
  source 1..3798: mol_type = other DNA
  gene 1..3798: note = Tair Locus: AT5G42390.1 (Isoform 1 of SPP of Arabidosis thaliana)
atggcttcatcgtcctcttccattttcaccggtgttaagttctctccgatcttagctccctttaactccggagatagccgccgctctcgatatctaaaagatagccggaataaagttaggtttaatccatcgtcgccgcgtctcactcctcatcgtgttcgcgtcgaagctccgtctttaattccctataatggtctttgggcggcgcagccaaattcgcataagggacgtttaaagaggaacattgtttcgggaaaagaagctaccgggatatcactttctcaaggacgtaatttctgtcttacttgtaaaaggaatcaagctggaatcagaagagcattacctagtgcctttgtggatagaactgcatttagtttgtcgaggtcgagtttgacatcttctctgaggaaacattctcagattgtgaatgcaactcttggaccagatgagccacatgctgctggtacagcttggcctgatggtattgttgcggagagacaagatctcgacttattgcctcctgagattgatagtgcagagctagaagcgtttcttggttgtgaacttccttctcatccaaagttgcaccggggtcaattgaaaaatgggcttcgatatcttattttgccaaacaaagttccaccgaacagatttgaggcacacatggaagttcatgtaggatcgattgatgaggaagaagatgagcaagggattgctcatatgatagaacatgttgctttccttgggagcaagaaacgtgagaaacttcttggtacaggtgcccgctctaatgcctacaccgatttccaccatacagtatttcatattcattctccaacccacacgaaggactctgaggatgacctcttcccatctgtgctcgatgccttaaatgagatagcctttcacccaaaatttctctcttctcgagttgagaaagaaaggcgggccatactttcagaacttcagatgatgaatacaattgagtatcgtgttgactgccagttgttgcaacatctacattctgagaacaagctgggtaggaggttccctattggattggaagagcagattaagaagtgggatgttgacaaaattagaaaatttcatgagcgatggtacttcccagccaatgccactctatatattgtgggagatattgacaacatacctcggatagtccacaatattgaagctgtgtttggaaaaaatggtttggataatgaatcaacacccagttctcctagtcctggtgccttcggtgccatggctaattttctcgttccaaagctgccagctggtcttggtggtaccttttcaaatgagaaaacaaatactgctgatcagtctaagatgattaaaagagaaagacatgcaattcgtcccccggttgagcataattggtcgcttcctggaacaagtgttgatctaaagcctccacagatatttaagcacgagcttcttcagaattttgcaatcaacatgttttgcaagatacctgttagcaaggttcagacatttggagacctgcgaaatgttctgatgaaaagaatatttctctctgctctacattttcgaattaatacgagatacaagagttcaaacccaccatttacgtctgttgaactggaccatagtgattcgggaagggaaggatgcactgttacgactcttactgtcaccgcagagccccaaaattggcaaaatgcagtcaaagttgctgtccaagaggttcgaagacttaaagaatttggtgtcaccaggggtgaacttacacgttacatggatgctcttctaaaagatagtgaacatcttgcagctatgatagataatgtgtcttctgttgataacctggattttatcatggaaagtgatgcactaagccacacggttatggatcagacgcaaggacatgagaccttggttgctgtagctggaactgttactcttgaagaggtaaacactgttggcgccaaggtgctagaatttatttctgatttcggaagacccactgcccctcttcccgcagcaatagttgcttgtgttcccactaaggtgcatgttgatggggttggtgagagtgatttcaatatatctcctgatgaaataatagagtctgtcaaatcaggattattagcacccattgaagctgagcctgagctcgaggtgccaaaagaattgatctctcaatcacaattgaaagagttaaccttgcagagaaatccatgttttgtccctattcctgggtcaggtttaaccaaattgcatgacaaagagactgggatcactcagcttcggttatccaatgggatagctgtaaattacaagaaatctacgaccgaatcccgggcaggtgtcatgcggctcattgttggtggtggacgagctgctgaaacatctgattccaaaggagctgtggttgttggtgttcgtacactcagcgagggtggtcgagttggagacttttcaagggagcaggtggagctgttctgtgtaaatcatttgataaactgttcgttggagtcaactgaggagttcattgccatggaattccggtttactctgagagacaatggaatgcaagcggcttttcagctgcttcacatggtgcttgagcgtagtgtctggttggaagatgcatttgatcgagcaaggcagctatatctttcgtattttcgatcaattcctaaaagcttggagcgtgcgactgctcataaacttatgatcgctatgctgaacggtgatgagcgttttgttgagccgacacctaagtcactacagagcttgaatctagaatctgtgaaagatgcggtcatgagtcattttgttggggacaatatggaggtaagcattgtgggggacttttcagaggaggaaatcgagcgttgcatccttgattaccttggtacagttaaagctagccatgattctgcaaaaccaccaggatctgagcctatcttgtttcgccaacctacagctggcttgcaatttcaacaggtattcttgaaggatactgatgagagagcatgtgcatacattgcgggaccagcgccaaatcgatggggctttacagttgacggggatgacctttttcagtctgttagcaaacttcctgttgctcatgatggacttttgaaatcggaggaacaattgctagagggaggagacagggagttgcaaaagaagcttcgtgcccaccctcttttctttggagttacaatgggtcttctggccgaaatcataaactcaaggcttttcacgacagtaagagattctcttggactgacatatgacgtatcatttgagctaaacctatttgataggctgaatctcgggtggtatgtgatatctgtgacttcaactcccggcaaggtatacaaagcggttgatgcctgtaagagtgttcttagaggtttgcatagcaaccaaattgcccctagggagttggatagggcaaagcgaactcttctaatgagacatgaagctgaactcaagtccaatgcctattggcttaatctattagctcacttgcaggcctcctctgttccgaggaaggaactgtcatgcattaaagaacttgtttcattgtatgaagctgcttcgatcgaggacatatacctagcttataatcaattaagagtggacgaggactctctgtactcctgtattggtattgctggggcacaggcgggtgaagagattactgttctctcagaagaggaagaaccagaagatgttttctcaggagttgttcctgtgggacgaggctcatcgatgaccacaagaccaacaacctga

SEQ ID NO: 2 - DNA, 3261 bp, Arabidopsis thaliana
  source 1..3261: mol_type = other DNA
  gene 1..3261: note = Tair Locus: AT5G42390.2 (Isoform 2 of SPP of Arabidopsis thaliana)
atggcttcatcgtcctcttccattttcaccggtgttaagttctctccgatcttagctccctttaactccggagatagccgccgctctcgatatctaaaagatagccggaataaagttaggtttaatccatcgtcgccgcgtctcactcctcatcgtgttcgcgtcgaagctccgtctttaattccctataatggtctttgggcggcgcagccaaattcgcataagggacgtttaaagaggaacattgtttcgggaaaagaagctaccgggatatcactttctcaaggacgtaatttctgtcttacttgtaaaaggaatcaagctggaatcagaagagcattacctagtgcctttgtggatagaactgcatttagtttgtcgaggtcgagtttgacatcttctctgaggaaacattctcagattgtgaatgcaactcttggaccagatgagccacatgctgctggtacagcttggcctgatggtattgttgcggagagacaagatctcgacttattgcctcctgagattgatagtgcagagctagaagcgtttcttggttgtgaacttccttctcatccaaagttgcaccggggtcaattgaaaaatgggcttcgatatcttattttgccaaacaaagttccaccgaacagatttgaggcacacatggaagttcatgtaggatcgattgatgaggaagaagatgagcaagggattgctcatatgatagaacatgttgctttccttgggagcaagaaacgtgagaaacttcttggtacaggtgcccgctctaatgcctacaccgatttccaccatacagtatttcatattcattctccaacccacacgaaggactctgaggatgacctcttcccatctgtgctcgatgccttaaatgagatagcctttcacccaaaatttctctcttctcgagttgagaaagaaaggcgggccatactttcagaacttcagatgatgaatacaattgagtatcgtgttgactgccagttgttgcaacatctacattctgagaacaagctgggtaggaggttccctattggattggaagagcagattaagaagtgggatgttgacaaaattagaaaatttcatgagcgatggtacttcccagccaatgccactctatatattgtgggagatattgacaacatacctcggatagtccacaatattgaagctgtgtttggaaaaaatggtttggataatgaatcaacacccagttctcctagtcctggtgccttcggtgccatggctaattttctcgttccaaagctgccagctggtcttggtggtaccttttcaaatgagaaaacaaatactgctgatcagtctaagatgattaaaagagaaagacatgcaattcgtcccccggttgagcataattggtcgcttcctggaacaagtgttgatctaaagcctccacagatatttaagcacgagcttcttcagaattttgcaatcaacatgttttgcaagatacctgttagcaaggttcagacatttggagacctgcgaaatgttctgatgaaaagaatatttctctctgctctacattttcgaattaatacgagatacaagagttcaaacccaccatttacgtctgttgaactggaccatagtgattcgggaagggaaggatgcactgttacgactcttactgtcaccgcagagccccaaaattggcaaaatgcagtcaaagttgctgtccaagaggttcgaagacttaaagaatttggtgtcaccaggggtgaacttacacgttacatggatgctcttctaaaagatagtgaacatcttgcagctatgatagataatgtgtcttctgttgataacctggattttatcatggaaagtgatgcactaagccacacggttatggatcagacgcaaggacatgagaccttggttgctgtagctggaactgttactcttgaagaggtaaacactgttggcgccaaggtgctagaatttatttctgatttcggaagacccactgcccctcttcccgcagcaatagttgcttgtgttcccactaaggtgcatgttgatggggttggtgagagtgatttcaatatatctcctgatgaaataatagagtctgtcaaatcaggattattagcacccattgaagctgagcctgagctcgaggtgccaaaagaattgatctctcaatcacaattgaaagagttaaccttgcagagaaatccatgttttgtccctattcctgggtcaggtttaaccaaattgcatgacaaagagactgggatcactcagcttcggttatccaatgggatagctgtaaattacaagaaatctacgaccgaatcccgggcaggtgtcatgcggctcattgttggtggtggacgagctgctgaaacatctgattccaaaggagctgtggttgttggtgttcgtacactcagcgagggtggtcgagttggagacttttcaagggagcaggtggagctgttctgtgtaaatcatttgataaactgttcgttggagtcaactgaggagttcattgccatggaattccggtttactctgagagacaatggaatgcaagcggcttttcagctgcttcacatggtgcttgagcgtagtgtctggttggaagatgcatttgatcgagcaaggcagctatatctttcgtattttcgatcaattcctaaaagcttggagcgtgcgactgctcataaacttatgatcgctatgctgaacggtgatgagcgttttgttgagccgacacctaagtcactacagagcttgaatctagaatctgtgaaagatgcggtcatgagtcattttgttggggacaatatggaggtaagcattgtgggggacttttcagaggaggaaatcgagcgttgcatccttgattaccttggtacagttaaagctagccatgattctgcaaaaccaccaggatctgagcctatcttgtttcgccaacctacagctggcttgcaatttcaacaggtattcttgaaggatactgatgagagagcatgtgcatacattgcgggaccagcgccaaatcgatggggctttacagttgacggggatgacctttttcagtctgttagcaaacttcctgttgctcatgatggacttttgaaatcggaggaacaattgctagagggaggagacagggagttgcaaaagaagcttcgtgcccaccctcttttctttggagttacaatgggtcttctggccgaaatcataaactcaagaatttgttga

SEQ ID NO: 3 - DNA, 3639 bp, synthetic construct
  source 1..3639: mol_type = other DNA
  misc_difference 1..2: note = Deletion of 54 aminoacids corresponding to the N-terminal chloroplast transit peptide. A methione was added as start codon
atggtagaagccccctcactcataccttacaacggactgtgggccgctcaaccgaacagtcacaaagggagactgaaacgcaatatagtgagcggtaaagaggcaactggaattagtctgtcccagggcagaaacttttgcctgacctgcaagagaaaccaggcggggattcgtcgggcgctgccctctgcgttcgtagaccgtacggccttctctctttccagatcaagcctgaccagttccttgagaaagcacagccaaatcgtaaacgctacactggggcccgacgaacctcacgccgccgggactgcctggcccgatgggattgtcgccgaacggcaagatctggatttgttgccaccagaaatcgacagcgccgaattggaagcctttctggggtgcgagctcccaagccaccccaaactccatagagggcagctcaagaacggattgcgttacctcatcctgccgaataaggtcccacctaatcggttcgaagcccatatggaagtgcacgtcggttccatcgacgaagaagaggacgaacagggcatcgcgcacatgattgagcacgtcgcctttctcggatccaagaagcgcgaaaagctgctcgggacaggggcaaggagcaacgcatatacagactttcatcacaccgttttccacatccactcccccactcataccaaagattccgaagacgatctgtttccttcagttttggacgcacttaacgaaatcgcattccatccgaagttcttgagctccagggtggaaaaggagcgccgagctatcttgagcgagctgcaaatgatgaacaccatcgaatacagagtggattgtcaacttctccagcacctgcacagcgaaaataaacttgggcggcgttttcccatcgggctcgaggaacaaatcaagaaatgggacgtagataagatacgaaagttccacgaacggtggtattttcccgctaacgctaccctgtacatcgtaggcgacatcgataatatcccccgcatcgtgcataacatcgaggccgtattcggcaagaacggcctggacaacgagtctacccctagctccccctctccaggagcttttggggcaatggccaacttcctggtacctaaactccctgccggcctcggcggaactttctctaacgaaaagactaacaccgcagaccaatccaaaatgatcaagcgcgagcggcacgctatccggcccccagtggaacacaactggtcactgcccggcaccagcgtcgaccttaaaccaccgcaaattttcaaacatgaactcctgcaaaacttcgcgataaatatgttctgtaaaatccccgtgtctaaagtacaaactttcggtgatcttagaaacgtgctcatgaagcgaattttcctgagcgccttgcacttccgtatcaacacccggtataaatccagtaatccccctttcacctcagtggagctcgatcactccgacagtggcagagagggctgtaccgtaacaacactgacagtgactgctgaaccacagaactggcagaacgccgtgaaggtggccgtacaggaagtgaggcgactcaaggagttcggcgttactagaggggagctgaccagatatatggacgccctgctcaaggacagcgagcacctggcggccatgattgacaacgtttcttccgtggacaatctcgacttcattatggagagcgacgctctctctcatacagtgatggaccaaactcaggggcacgaaacactcgtggccgtcgcaggcacagtcacgctggaagaagtgaatacagtaggggcaaaagtccttgagttcatcagcgactttggccggcctaccgcgccactccctgccgccatcgtggcatgcgtgccaaccaaagtacacgtggacggtgtcggggaatcagactttaacatcagcccagacgagatcatcgaaagcgttaagagtgggctcttggctccgatcgaggcggaaccagaactggaagtcccgaaggagctcattagtcagagccagcttaaggaactcactctgcaacggaacccctgcttcgtgccaataccaggtagtggcctgacaaagctgcacgataaggaaaccggaattacacaactgcgacttagcaacggcatcgcggtcaactataagaagagcaccactgagagcagagctggggttatgcgccttatagtgggggggggtcgggcagcggagactagcgacagtaaaggggcggtcgtggttggggtgagaacgctgtccgaaggcggaagagtgggtgatttcagccgagaacaagttgaactcttttgcgttaaccaccttatcaattgcagtctggaatctaccgaagaatttatagctatggagtttcgcttcaccttgcgggataacgggatgcaggctgccttccaactgctccatatggtcttggaacgctcagtttggctcgaggacgctttcgaccgcgctagacaactctacctgtcttacttccgttccatccccaagtccctggaaagggccaccgcgcacaagttgatgatagcaatgttgaatggagacgaaaggttcgtggaacccaccccaaaatccctgcaatcactgaaccttgagtcagttaaggacgcagtgatgagccacttcgtgggcgataacatggaagtgtctatcgtcggagatttctccgaagaagagattgaaagatgtattctcgactatctcggcaccgtgaaggcatctcacgacagtgccaagccacccggcagcgaaccaattctgttcaggcagcccactgcagggctgcagttccagcaagtctttctgaaagacaccgacgaacgggcttgcgcttatatcgctggccccgctcctaacaggtggggtttcactgtggatggagacgatttgttccaatccgtgtctaagctgccagtggcacacgacgggctgctgaagtctgaagagcagctcctggaaggcggggatcgggaactgcagaagaaactccgcgcacatcccctgttcttcggtgtgaccatggggctgctcgcagagataatcaattcccgactgtttactaccgtgagggactccctgggccttacctacgatgtgtccttcgaactgaatttgttcgacagattgaacctgggctggtacgttatcagcgtcaccagtacccctggaaaagtgtataaggccgtggacgcttgcaaatctgtgctgcgtgggctgcactctaatcagatcgcaccccgggaactggaccgcgctaaacggacactgctgatgaggcacgaggcggagcttaaaagcaacgcttactggctgaacctgctggcacatctccaagcaagcagtgtaccaagaaaagagttgtcttgtatcaaggagctggtgagcctgtacgaggcagccagtattgaagatatttatcttgcctacaaccagctgcgcgttgatgaagatagtctctatagttgcatcggcatcgccggtgcccaagcaggcgaggaaatcacggtactttctgaggaagaggagcccgaggacgtcttttctggcgtggttcccgtcgggcgcgggagctcaatgacaacccgcccgactacataa

SEQ ID NO: 4 - DNA, 4401 bp, synthetic construct
  source 1..4401: mol_type = other DNA
  misc_feature 1..2: note = GFP was fused to the N-terminal of SPP. SPP has a deletion of 54 aminoacids corresponding to the N-terminal chloroplast transit peptide. No methione added as the start codon comes from GFP.
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctctacaaaagcggttccggaccggatcaaacaagtttgtacaaaaaagcaggctgggtagaagccccctcactcataccttacaacggactgtgggccgctcaaccgaacagtcacaaagggagactgaaacgcaatatagtgagcggtaaagaggcaactggaattagtctgtcccagggcagaaacttttgcctgacctgcaagagaaaccaggcggggattcgtcgggcgctgccctctgcgttcgtagaccgtacggccttctctctttccagatcaagcctgaccagttccttgagaaagcacagccaaatcgtaaacgctacactggggcccgacgaacctcacgccgccgggactgcctggcccgatgggattgtcgccgaacggcaagatctggatttgttgccaccagaaatcgacagcgccgaattggaagcctttctggggtgcgagctcccaagccaccccaaactccatagagggcagctcaagaacggattgcgttacctcatcctgccgaataaggtcccacctaatcggttcgaagcccatatggaagtgcacgtcggttccatcgacgaagaagaggacgaacagggcatcgcgcacatgattgagcacgtcgcctttctcggatccaagaagcgcgaaaagctgctcgggacaggggcaaggagcaacgcatatacagactttcatcacaccgttttccacatccactcccccactcataccaaagattccgaagacgatctgtttccttcagttttggacgcacttaacgaaatcgcattccatccgaagttcttgagctccagggtggaaaaggagcgccgagctatcttgagcgagctgcaaatgatgaacaccatcgaatacagagtggattgtcaacttctccagcacctgcacagcgaaaataaacttgggcggcgttttcccatcgggctcgaggaacaaatcaagaaatgggacgtagataagatacgaaagttccacgaacggtggtattttcccgctaacgctaccctgtacatcgtaggcgacatcgataatatcccccgcatcgtgcataacatcgaggccgtattcggcaagaacggcctggacaacgagtctacccctagctccccctctccaggagcttttggggcaatggccaacttcctggtacctaaactccctgccggcctcggcggaactttctctaacgaaaagactaacaccgcagaccaatccaaaatgatcaagcgcgagcggcacgctatccggcccccagtggaacacaactggtcactgcccggcaccagcgtcgaccttaaaccaccgcaaattttcaaacatgaactcctgcaaaacttcgcgataaatatgttctgtaaaatccccgtgtctaaagtacaaactttcggtgatcttagaaacgtgctcatgaagcgaattttcctgagcgccttgcacttccgtatcaacacccggtataaatccagtaatccccctttcacctcagtggagctcgatcactccgacagtggcagagagggctgtaccgtaacaacactgacagtgactgctgaaccacagaactggcagaacgccgtgaaggtggccgtacaggaagtgaggcgactcaaggagttcggcgttactagaggggagctgaccagatatatggacgccctgctcaaggacagcgagcacctggcggccatgattgacaacgtttcttccgtggacaatctcgacttcattatggagagcgacgctctctctcatacagtgatggaccaaactcaggggcacgaaacactcgtggccgtcgcaggcacagtcacgctggaagaagtgaatacagtaggggcaaaagtccttgagttcatcagcgactttggccggcctaccgcgccactccctgccgccatcgtggcatgcgtgccaaccaaagtacacgtggacggtgtcggggaatcagactttaacatcagcccagacgagatcatcgaaagcgttaagagtgggctcttggctccgatcgaggcggaaccagaactggaagtcccgaaggagctcattagtcagagccagcttaaggaactcactctgcaacggaacccctgcttcgtgccaataccaggtagtggcctgacaaagctgcacgataaggaaaccggaattacacaactgcgacttagcaacggcatcgcggtcaactataagaagagcaccactgagagcagagctggggttatgcgccttatagtgggggggggtcgggcagcggagactagcgacagtaaaggggcggtcgtggttggggtgagaacgctgtccgaaggcggaagagtgggtgatttcagccgagaacaagttgaactcttttgcgttaaccaccttatcaattgcagtctggaatctaccgaagaatttatagctatggagtttcgcttcaccttgcgggataacgggatgcaggctgccttccaactgctccatatggtcttggaacgctcagtttggctcgaggacgctttcgaccgcgctagacaactctacctgtcttacttccgttccatccccaagtccctggaaagggccaccgcgcacaagttgatgatagcaatgttgaatggagacgaaaggttcgtggaacccaccccaaaatccctgcaatcactgaaccttgagtcagttaaggacgcagtgatgagccacttcgtgggcgataacatggaagtgtctatcgtcggagatttctccgaagaagagattgaaagatgtattctcgactatctcggcaccgtgaaggcatctcacgacagtgccaagccacccggcagcgaaccaattctgttcaggcagcccactgcagggctgcagttccagcaagtctttctgaaagacaccgacgaacgggcttgcgcttatatcgctggccccgctcctaacaggtggggtttcactgtggatggagacgatttgttccaatccgtgtctaagctgccagtggcacacgacgggctgctgaagtctgaagagcagctcctggaaggcggggatcgggaactgcagaagaaactccgcgcacatcccctgttcttcggtgtgaccatggggctgctcgcagagataatcaattcccgactgtttactaccgtgagggactccctgggccttacctacgatgtgtccttcgaactgaatttgttcgacagattgaacctgggctggtacgttatcagcgtcaccagtacccctggaaaagtgtataaggccgtggacgcttgcaaatctgtgctgcgtgggctgcactctaatcagatcgcaccccgggaactggaccgcgctaaacggacactgctgatgaggcacgaggcggagcttaaaagcaacgcttactggctgaacctgctggcacatctccaagcaagcagtgtaccaagaaaagagttgtcttgtatcaaggagctggtgagcctgtacgaggcagccagtattgaagatatttatcttgcctacaaccagctgcgcgttgatgaagatagtctctatagttgcatcggcatcgccggtgcccaagcaggcgaggaaatcacggtactttctgaggaagaggagcccgaggacgtcttttctggcgtggttcccgtcgggcgcgggagctcaatgacaacccgcccgactacataa

SEQ ID NO: 5 - DNA, 1302 bp, synthetic construct
  source 1..1302: mol_type = other DNA
  gene 1..2: note = GenBank: AF054182.1
atggaaacaagagtaacatgtttagaaagtggactcagagtagcttcggaagactctgggctctcaacatgcacagttggactctggattgatgctggaagtagatacgaaaatgagaagaacaatggaacagcacactttctggagcatatggctttcaagggcaccaagaagagatcccagttagatctggaacttgagattgaaaatatgggtgctcatctcaatgcctatacctccagagagcagactgtatactatgccaaagcattctctaaagacttgccaagagctgtagaaattcttgctgatataatacaaaacagcacattgggagaagcagagattgaacgtgagcgtggagtaatccttagagagatgcaggaagttgaaaccaatttacaagaagttgtttttgattatcttcatgccacagcttatcaaaatactgcacttggacggacaattttgggaccaactgaaaatatcaaatctataagtcgtaaggacttagtggattatataaccacacattataaggggccaagaatagtgcttgctgctgctggaggtgtttcccatgatgaattgcttgacttagcaaagtttcatttcggtgactctttatgcacacacaaaggagaaataccagctctgcctccctgcaaattcacaggaagtgagattcgtgtgagggatgacaagatgcctttggcgcaccttgcaatagctgttgaagctgttggttgggcacatccagatacaatctgtctcatggttgcaaacacgctgattggcaactgggatcgctcttttgggggaggaatgaatttatctagcaagctggcccagctcacttgtcatggcaatctttgccatagctttcagtctttcaacacttcctacacagatacaggattatggggactgtatatggtttgtgaatcatccactgttgcagacatgctacatgttgttcaaaaagaatggatgcgactctgtacaagtgtcacagaaagtgaggttgcacgagccagaaatcttctgaaaacaaacatgttgttgcagcttgatggttcaactccaatttgtgaagatattggtaggcaaatgttatgctataatagaaggattcccatccctgagcttgaagcaagaattgatgctgtgaatgctgagacaattcgagaagtatgtaccaaatacatttataataggagtccagctattgctgctgttggtcccattaagcaactaccagattttaaacagatacgcagtaacatgtgttggcttcgtgat

SEQ ID NO: 6 - DNA, 1590 bp, synthetic construct
  source 1..1590: mol_type = other DNA
  unsure 1..1590: note = NCBI Reference Sequence: XM_005266059.4
atgagtggtggtgcctatcccaacatccccctctcttctcccttacctggagtacccaagcctgtttttgctacagttgatggacaggaaaagtttgaaaccaaagtaaccacattggataatgggcttcgcgtggcatctcagaataagtttggacagttttgtacagtaggaattcttatcaattcaggatcgagatatgaagcgaaataccttagtggaattgctcactttttggaaaaattggcattttcgtctactgctcgatttgacagcaaagatgaaattctgcttacgttggaaaagcatgggggtatctgtgactgccagacatcaagagacaccaccatgtatgctgtgtctgctgatagcaaaggcttggacacggtggttgccttactggctgatgtggttctgcagccccggctaacagatgaagaagtcgagatgacgcggatggcggtccagtttgagctggaggacctgaacctgcggcctgacccagagccacttctcaccgagatgattcatgaagcggcttacagggagaacacagttggcctccaccgtttctgccccacagaaaacgtagcaaagatcaaccgagaggtgctgcattcctacctgaggaactactacactcccgaccgcatggtgctggccggcgtgggcgtggagcacgagcatctggtggactgtgcccggaagtacctcctgggggtccagccggcctgggggagcgcagaggccgtggatattgacagatctgtggcccagtacactggggggattgccaagctagaaagagacatgtccaatgtcagcctgggcccgacccccatccccgagctcacgcacatcatggttggactggagagctgctccttcctggaggaggacttcatcccctttgcagtgttgaacatgatgatgggcggaggtggctccttctcggctggtgggcccggcaagggcatgttctccaggctctacctcaacgtgctcaacaggcaccactggatgtataacgcgacctcctaccaccacagctacgaggacactggcctcctttgcatccatgccagcgccgacccaagacaggttcgagaaatggtagaaatcatcacaaaggagtttattttaatgggcggaaccgtggacacggtggagctggaacgagccaagacgcagctgacatcaatgctcatgatgaacctggaatccaggcctgtgatcttcgaggatgtggggaggcaggtgctggccactcgctccagaaagctgccgcacgagctgtgcacgctcatccgtgagtaccgcaggggtagtgaggggctgccgcaggcctcggccaggctcagaggaggccgtctcgccctcccgcaggccgtggtgggcctgtggtatgtccatcacacccagaatctggggccttcaccagttgtcctcagcaggggcggccaagggcagggtcgtggggtcgcagacctggtctcacttcccggccccaccatgcacctgctgcctccttggcaggtgaccccaccctctgcacccctgggaggggctgtcactactgcccagagttgt

SEQ ID NO: 7 - DNA, 1470 bp, Homo sapiens
  source 1..1470: mol_type = other DNA
  gene 1..1470: note = GenBank: AF054182.1
atggcggctgcggcggctcgagtggtgttgtcatccgcggcgcgaggcggcctctggggtttcagcgagagtcttctaatccgaggcgctgcgggacggtcattatattttggagagaacagattaagaagtacacaggctgctacccaagttgttctgaatgttcctgaaacaagagtaacatgtttagaaagtggactcagagtagcttcggaagactctgggctctcaacatgcacagttggactctggattgatgctggaagtagatacgaaaatgagaagaacaatggaacagcacactttctggagcatatggctttcaagggcaccaagaagagatcccagttagatctggaacttgagattgaaaatatgggtgctcatctcaatgcctatacctccagagagcagactgtatactatgccaaagcattctctaaagacttgccaagagctgtagaaattcttgctgatataatacaaaacagcacattgggagaagcagagattgaacgtgagcgtggagtaatccttagagagatgcaggaagttgaaaccaatttacaagaagttgtttttgattatcttcatgccacagcttatcaaaatactgcacttggacggacaattttgggaccaactgaaaatatcaaatctataagtcgtaaggacttagtggattatataaccacacattataaggggccaagaatagtgcttgctgctgctggaggtgtttcccatgatgaattgcttgacttagcaaagtttcatttcggtgactctttatgcacacacaaaggagaaataccagctctgcctccctgcaaattcacaggaagtgagattcgtgtgagggatgacaagatgcctttggcgcaccttgcaatagctgttgaagctgttggttgggcacatccagatacaatctgtctcatggttgcaaacacgctgattggcaactgggatcgctcttttgggggaggaatgaatttatctagcaagctggcccagctcacttgtcatggcaatctttgccatagctttcagtctttcaacacttcctacacagatacaggattatggggactgtatatggtttgtgaatcatccactgttgcagacatgctacatgttgttcaaaaagaatggatgcgactctgtacaagtgtcacagaaagtgaggttgcacgagccagaaatcttctgaaaacaaacatgttgttgcagcttgatggttcaactccaatttgtgaagatattggtaggcaaatgttatgctataatagaaggattcccatccctgagcttgaagcaagaattgatgctgtgaatgctgagacaattcgagaagtatgtaccaaatacatttataataggagtccagctattgctgctgttggtcccattaagcaactaccagattttaaacagatacgcagtaacatgtgttggcttcgtgattaa

SEQ ID NO: 8 - DNA, 1692 bp, Homo sapiens
  source 1..1692: mol_type = other DNA
  unsure 1..1692: note = NCBI Reference Sequence: XM_005266059.4
atggcggctgtggtgctggcggcgacgcggttgctgcggggctcgggttcttggggctgttcgcggctgaggtttggacctcctgcgtacagacggtttagtagtggtggtgcctatcccaacatccccctctcttctcccttacctggagtacccaagcctgtttttgctacagttgatggacaggaaaagtttgaaaccaaagtaaccacattggataatgggcttcgcgtggcatctcagaataagtttggacagttttgtacagtaggaattcttatcaattcaggatcgagatatgaagcgaaataccttagtggaattgctcactttttggaaaaattggcattttcgtctactgctcgatttgacagcaaagatgaaattctgcttacgttggaaaagcatgggggtatctgtgactgccagacatcaagagacaccaccatgtatgctgtgtctgctgatagcaaaggcttggacacggtggttgccttactggctgatgtggttctgcagccccggctaacagatgaagaagtcgagatgacgcggatggcggtccagtttgagctggaggacctgaacctgcggcctgacccagagccacttctcaccgagatgattcatgaagcggcttacagggagaacacagttggcctccaccgtttctgccccacagaaaacgtagcaaagatcaaccgagaggtgctgcattcctacctgaggaactactacactcccgaccgcatggtgctggccggcgtgggcgtggagcacgagcatctggtggactgtgcccggaagtacctcctgggggtccagccggcctgggggagcgcagaggccgtggatattgacagatctgtggcccagtacactggggggattgccaagctagaaagagacatgtccaatgtcagcctgggcccgacccccatccccgagctcacgcacatcatggttggactggagagctgctccttcctggaggaggacttcatcccctttgcagtgttgaacatgatgatgggcggaggtggctccttctcggctggtgggcccggcaagggcatgttctccaggctctacctcaacgtgctcaacaggcaccactggatgtataacgcgacctcctaccaccacagctacgaggacactggcctcctttgcatccatgccagcgccgacccaagacaggttcgagaaatggtagaaatcatcacaaaggagtttattttaatgggcggaaccgtggacacggtggagctggaacgagccaagacgcagctgacatcaatgctcatgatgaacctggaatccaggcctgtgatcttcgaggatgtggggaggcaggtgctggccactcgctccagaaagctgccgcacgagctgtgcacgctcatccgtgagtaccgcaggggtagtgaggggctgccgcaggcctcggccaggctcagaggaggccgtctcgccctcccgcaggccgtggtgggcctgtggtatgtccatcacacccagaatctggggccttcaccagttgtcctcagcaggggcggccaagggcagggtcgtggggtcgcagacctggtctcacttcccggccccaccatgcacctgctgcctccttggcaggtgaccccaccctctgcacccctgggaggggctgtcactactgcccagagttgttaa

SEQ ID NO: 9 - DNA, 51 bp, synthetic construct
  source 1..51: mol_type = other DNA
ggcggaggtggctccttctcggctggtgggcccggcaagggcatgttctcc

SEQ ID NO: 10 - DNA, 1758 bp, synthetic construct
  source 1..1758: mol_type = other DNA
  unsure 1..1758: note = Truncated version of SPP (AT5G42390.1) residues 122 to 707
actgcatttagtttgtcgaggtcgagtttgacatcttctctgaggaaacattctcagattgtgaatgcaactcttggaccagatgagccacatgctgctggtacagcttggcctgatggtattgttgcggagagacaagatctcgacttattgcctcctgagattgatagtgcagagctagaagcgtttcttggttgtgaacttccttctcatccaaagttgcaccggggtcaattgaaaaatgggcttcgatatcttattttgccaaacaaagttccaccgaacagatttgaggcacacatggaagttcatgtaggatcgattgatgaggaagaagatgagcaagggattgctcatatgatagaacatgttgctttccttgggagcaagaaacgtgagaaacttcttggtacaggtgcccgctctaatgcctacaccgatttccaccatacagtatttcatattcattctccaacccacacgaaggactctgaggatgacctcttcccatctgtgctcgatgccttaaatgagatagcctttcacccaaaatttctctcttctcgagttgagaaagaaaggcgggccatactttcagaacttcagatgatgaatacaattgagtatcgtgttgactgccagttgttgcaacatctacattctgagaacaagctgggtaggaggttccctattggattggaagagcagattaagaagtgggatgttgacaaaattagaaaatttcatgagcgatggtacttcccagccaatgccactctatatattgtgggagatattgacaacatacctcggatagtccacaatattgaagctgtgtttggaaaaaatggtttggataatgaatcaacacccagttctcctagtcctggtgccttcggtgccatggctaattttctcgttccaaagctgccagctggtcttggtggtaccttttcaaatgagaaaacaaatactgctgatcagtctaagatgattaaaagagaaagacatgcaattcgtcccccggttgagcataattggtcgcttcctggaacaagtgttgatctaaagcctccacagatatttaagcacgagcttcttcagaattttgcaatcaacatgttttgcaagatacctgttagcaaggttcagacatttggagacctgcgaaatgttctgatgaaaagaatatttctctctgctctacattttcgaattaatacgagatacaagagttcaaacccaccatttacgtctgttgaactggaccatagtgattcgggaagggaaggatgcactgttacgactcttactgtcaccgcagagccccaaaattggcaaaatgcagtcaaagttgctgtccaagaggttcgaagacttaaagaatttggtgtcaccaggggtgaacttacacgttacatggatgctcttctaaaagatagtgaacatcttgcagctatgatagataatgtgtcttctgttgataacctggattttatcatggaaagtgatgcactaagccacacggttatggatcagacgcaaggacatgagaccttggttgctgtagctggaactgttactcttgaagaggtaaacactgttggcgccaaggtgctagaatttatttctgatttcggaagacccactgcccctcttcccgcagcaatagttgcttgtgttcccactaaggtgcatgttgatggggttggtgagagtgatttcaatatatctcctgatgaaataatagagtctgtc

SEQ ID NO: 11 - DNA, 780 bp, synthetic construct
  source 1..780: mol_type = other DNA
  unsure 1..780: note = Truncated version of SPP (AT5G42390.1) residues 1004 to 1263
gatactgatgagagagcatgtgcatacattgcgggaccagcgccaaatcgatggggctttacagttgacggggatgacctttttcagtctgttagcaaacttcctgttgctcatgatggacttttgaaatcggaggaacaattgctagagggaggagacagggagttgcaaaagaagcttcgtgcccaccctcttttctttggagttacaatgggtcttctggccgaaatcataaactcaaggcttttcacgacagtaagagattctcttggactgacatatgacgtatcatttgagctaaacctatttgataggctgaatctcgggtggtatgtgatatctgtgacttcaactcccggcaaggtatacaaagcggttgatgcctgtaagagtgttcttagaggtttgcatagcaaccaaattgcccctagggagttggatagggcaaagcgaactcttctaatgagacatgaagctgaactcaagtccaatgcctattggcttaatctattagctcacttgcaggcctcctctgttccgaggaaggaactgtcatgcattaaagaacttgtttcattgtatgaagctgcttcgatcgaggacatatacctagcttataatcaattaagagtggacgaggactctctgtactcctgtattggtattgctggggcacaggcgggtgaagagattactgttctctcagaagaggaagaaccagaagatgttttctcaggagttgttcctgtgggacgaggctcatcgatgaccacaagacca

SEQ ID NO: 12 - DNA, 246 bp, synthetic construct
  source 1..246: mol_type = other DNA
  unsure 1..246: note = Zn2+-binding region (HxxEHx76E motif ) from SPP (AT5G42390.1)
catatgatagaacatgttgctttccttgggagcaagaaacgtgagaaacttcttggtacaggtgcccgctctaatgcctacaccgatttccaccatacagtatttcatattcattctccaacccacacgaaggactctgaggatgacctcttcccatctgtgctcgatgccttaaatgagatagcctttcacccaaaatttctctcttctcgagttgagaaagaaaggcgggccatactttcagaa

SEQ ID NO: 13 - DNA, 243 bp, synthetic construct
  source 1..243: mol_type = other DNA
  unsure 1..243: note = Zn2+-binding region (HxxEHx76E motif ) from HUMAN Mitochondrial-processing peptidase subunit beta
cactttctggagcatatggctttcaagggcaccaagaagagatcccagttagatctggaacttgagattgaaaatatgggtgctcatctcaatgcctatacctccagagagcagactgtatactatgccaaagcattctctaaagacttgccaagagctgtagaaattcttgctgatataatacaaaacagcacattgggagaagcagagattgaacgtgagcgtggagtaatccttagagag

SEQ ID NO: 14 - DNA, 2847 bp, Homo sapiens
  source 1..2847: mol_type = other DNA
  gene 1..2847: note = GenBank: AY049784.1
atgctgaggagagtcactgttgctgcagtctgtgccacccggaggaagttgtgtgaggccgggcgggagctcgcggcgctctggggaatcgaaacgcggggtcggtgcgaagactctgctgctgccagaccctttcctattctggccatgcctggaaggaacaaggcgaagtctacctgcagctgccctgacctgcagcccaatggacaggatctgggcgagaacagccgggttgcccgtctaggagcggatgaatctgaggaagagggacggagggggtctctcagtaatgctggggaccctgagatcgtcaagtctcccagcgaccccaagcaataccgatacatcaaattacagaatggcttgcaggcacttctgatttcagacctaagtaatatggaaggtaaaacaggaaatacaacagatgatgaagaagaaggggaggtggaggaagaagaagatgatgatgaagattctggagctgaaatagaagatgacgatgaagagggttttgatgatgaagatgagtttgatgatgaacatgatgatgatcttgatactgaggataatgaattggaagaattagaagagagagcagaagctagaaaaaaaactactgaaaaacagtctgcagcggctctttgtgttggagttgggagtttcgctgatccagatgacctgccggggctggcacactttttggagcacatggtattcatgggtagtttgaaatatccagatgagaatggatttgatgccttcctgaagaagcatgggggtagtgataatgcctcaactgattgtgaacgcactgtctttcagtttgatgtccagaggaagtacttcaaggaagctcttgatagatgggcgcagttcttcatccacccactaatgatcagagatgcaattgaccgtgaagttgaagctgttgatagtgaatatcaacttgcaaggccttctgatgcaaacagaaaggaaatgttgtttggaagccttgctagacctggacatcctatgggaaaatttttttggggaaatgctgagacgctcaagcatgagccaagaaagaataatattgatacacatgctagattgagagaattctggatgcgttactactcttctcattacatgactttagtggttcaatccaaagaaacactggatactttggaaaagtgggtgactgaaatcttctctcagataccaaacaatgggttacccagaccaaactttggccatttaacggatccatttgacacaccagcatttaacaaactttatagagttgttccaatcagaaaaattcatgctctgaccatcacatgggcacttcctcctcaacagcaacattacagggtgaagccacttcattatatatcctggctggttggacatgaaggcaaaggcagcattctttctttccttaggaaaaaatgctgggctcttgcactgtttggtggaaatggtgagacaggatttgagcaaaattctacttattcagtgttcagcatttctattacattgactgatgagggttatgaacatttttatgaggttgcttacactgtctttcagtatttaaaaatgctgcagaagctaggcccagaaaaaagaatttttgaagagattcggaaaattgaggataatgaatttcattaccaagaacagacagatccagttgagtatgtggaaaacatgtgtgagaacatgcagctgtacccattgcaggacattctcactggagatcagcttctttttgaatacaagccagaagtcattggtgaagccttgaatcagctagttcctcaaaaagcaaatcttgttttactgtctggtgctaatgagggaaaatgtgacctcaaggagaaatggtttggaactcaatatagtatagaagatattgaaaactcttgggctgaactgtggaatagtaatttcgaattaaatccagatcttcatcttccagctgaaaacaagtacatagccacggactttacgttgaaggctttcgattgcccggaaacagaatacccagttaaaattgtgaatactccacaaggttgcctgtggtataagaaagacaacaaattcaaaatccccaaagcatatatacgtttccatctaatttcaccgttgatacagaaatctgcagcaaatgtggtcctctttgatatctttgtcaatatccttacgcataaccttgcggaaccagcttatgaagcagatgtggcacagctggagtataaactggtagctggagaacatggtttaattattcgagtgaaaggatttaaccacaaactacctctactgtttcagctcattattgactacttagctgagttcaattccacaccagctgtctttacaatgataactgagcagttgaagaagacctactttaacatcctcatcaagcccgagactttggccaaagatgtacggcttttaatcttggaatatgcccgttggtctatgattgacaagtaccaggctttgatggacggcctttcccttgagtctctgctgagcttcgtcaaagaattcaaatcccagctctttgtggagggcctggtacaagggaatgtcacaagcacagaatctatggatttcctgaaatatgttgttgacaaactaaacttcaagcctctggagcaggagatgcctgtgcagttccaggtggtagagctgcccagtggccaccatctatgcaaagtgaaagctctgaacaagggtgatgccaactctgaagtcactgtgtactaccaggtcaggtaccaggagtctaagagaatatacgcttatggagctgcttgtgatgcacatggaagaaccttgttttga

SEQ ID NO: 15 - DNA, 2244 bp, Homo sapiens
  source 1..2244: mol_type = other DNA
  gene 1..2244: note = GenBank: AK302616.1
atggaaggtaaaacaggaaatacaacagatgatgaagaagaagaggaggtggaggaagaagaagaagatgatgatgaagattctggagctgaaatagaagatgacgatgaagagggttttgatgatgaagatgagtttgatgatgaacatgatgatgatcttgatactgaggataatgaattggaagaattagaagagagagcagaagctagaaaaaaaactactgaaaaacagtctgcagcggctctttgtgttggagttgggagtttcgctgatccagatgacctgccggggctggcacactttttggagcacatggtattcatgggtagtttgaaatatccagatgagaatggatttgatgccttcctgaagaagcatgggggtagtgataatgcctcaactgattgtgaacgcactgtctttcagtttgatgtccagaggaagtacttcaaggaagctcttgatagatgggcgcagttcttcatccacccactaatgatcagagatgcaattgaccgtgaagttgaagctgctgatagtgaatatcaacttgcaaggccttctgatgcaaacagaaaggaaatgttgtttggaagccttgctagacctggacatcctatgggaaaatttttttggggaaatgctgagacgctcaagcatgagccaagaaagaataatattgatacacatgctagattgagagaattctggatgcgttactactcttctcattacatgactttagtggttcaatccaaagaaacactggatactttggaaaagtgggtgactgaaatcttctctcagataccaaacaatgggttacccagaccaaactttggccatttaacggatccatttgacacaccagcatttaacaaactttatagagttgttccaatcagaaaaattcatgctctgaccatcacatgggcacttcctcctcaacagcaacattacagggtgaagccacttcattatatatcctggctggttggacatgaaggcaaaggcagcattctttctttccttaggaaaaaatgctgggctcttgcactgtttggtggaaatggtgagacaggatttgagcaaaattctacttattcagtgttcagcatttctattacattgactgatgagggttatgaacatttttatgaggttgcttacactgtctttcagtatttaaaaatgctgcagaagctaggcccagaaaaaagaatttttgaagagattcggaaaattgaggataatgaatttcattaccaagaacagacagatccagttgagtatgtggaaaacatgtgtgagaacatgcagctgtacccattgcaggacattctcactggagatcagcttctttttgaatacaagccagaagtcattggtgaagccttgaatcagctagttcctcaaaaagcaaatcttgttttactgtctggtgctaatgagggaaaatgtgacctcaaggagaaatggtttggaactcaatatagtatagaagatattgaaaactcttgggctgaactgtggaatagtaatttcgaattaaatccagatcttcatcttccagctgaaaacaagtacatagccacggactttacgttgaaggctttcgattgcccggaaacagaatacccagttaaaattgtgaatactccacaaggttgcctgtggtataagaaagacaacaaattcaaaatccccaaagcatatatacgtttccatctaatttcaccgttgatacagaaatctgcagcaaatgtggtcctctttgatatctttgtcaatatccttacgcataaccttgcggaaccagcttatgaagcagatgtggcacagctggagtataaactggtagctggagaacatggtttaattattcgagtgaaaggatttaaccacaaactacctctactgtttcagctcattattgactacttagctgagttcaattccacaccagctgtctttacaatgataactgagcagttgaagaagacctactttaacatcctcatcaagcccgagactttggccaaagatgtacggcttttaatcctggaatatgcccgttggtctatgattgacaagtaccaggctttgatggacggcctttcccttgagtctctgctgagcttcgtcaaagaattcaaatcccagctctttgtggagggcctggtacaagggaatgtcacaagcacagttctatgtcaatgggcccaggattctgaaacatag

SEQ ID NO: 16 - DNA, 3066 bp, Homo sapiens
  source 1..3066: mol_type = other DNA
atgtggcgctgcggcgggcggcagggcctgtgtgtgctgaggcggctgagcggcggacatgcacaccacagagcgtggcgatggaacagtaaccgggcttgtgagagggctctgcagtataaactaggagacaagatccatggattcaccgtaaaccaggtgacatctgttcccgagctgttcctgactgcagtgaagctcacccatgatgacacaggagccaggtatttacacctggccagagaagacacgaataatctgttcagcgtgcagttccgtactactcccatggacagtactggtgttcctcacattcttgagcataccgtcctttgtgggtctcagaaatatccgtgcagagaccctttcttcaaaatgttgaaccggtccctctccacgttcatgaacgccttcacaggactttcagaatctcctctcggtgtatttggatgccacctttttcccatgtttacgcgagctggatttctggaaggatggcggctggaacatgagaatccgagcgacccccagacgcccttggtctttaaaggagtcgtctttaatgagatgaagggagcgtttacagacaatgagaggatattctcccagcaccttcagaacagacttcttcctgaccacacgtactcagtggtctccgggggtgacccactgtgcatcccggagcttacatgggagcagcttaagcagtttcatgccactcactatcacccaagcaatgctaggttcttcacgtacggtaattttccattagaacagcatctgaaacaaattcacgaggaagcactgagcaaattccagaaaattgaaccaagcaccgtggtgccagctcagacaccctgggacaagcctagggaattccagataacatgtggcccggattcatttgctacagatccctctaaacaaacaaccatcagcgttagcttcctcttaccggacatcaccgacacatttgaagccttcacattaagtcttctgtcttcactcttgacttctgggcccaattctcccttttacaaagccttgattgaatctggccttggcacagacttttctcctgatgttggatataatggctacacgagggaggcctactttagtgtcggcctccaagggattgcggagaaagacattgagaccgtcagaagcctcatagacagaacgattgatgaagtagttgagaaaggatttgaagatgatcgaattgaggctttacttcataaaattgaaatacagatgaaacatcagtctaccagctttgggctgatgctgacatcatacatagcttcttgctggaaccatgatggggaccctgtggagctcttgaagttgggaaatcagttagctaaattcagacagtgcctgcaggaaaatccaaaatttttgcaagaaaaagtaaaacagtattttaagaataaccagcataagctgactttatcgatgaggccagatgacaagtatcacgagaagcaggcacaggtggaagccacgaagctcaagcagaaggtcgaggctctgtcccccggagacaggcagcagatctacgagaaaggtctagaattacggagtcaacaaagcaaacctcaagatgcctcttgtctgccagcgttgaaagtttccgatattgaacccaccatacctgtcacagagttggacgtggtcctgacagctggagatatccctgttcagtactgcgcccagcccaccaatggcatggtgtatttccgggccttctccagcctgaacacactccccgaggagctgaggccctatgtgcccctcttctgcagcgtcctcaccaagctgggctgcggccttcttgactaccgggagcaggctcagcagatagaattgaagaccggagggatgagtgcttctccccacgtgctccccgacgactcacacatggacacctacgagcagggtgtgcttttctcctctctctgcctggatcgaaacctgccagacatgatgcagctatggagtgaaatatttaacaacccgtgctttgaagaagaggagcacttcaaggtgctggtgaagatgaccgcccaggagctcgccaatggaattcctgactctgggcacctgtacgcatccatcagggcaggccggaccctcacgcccgcaggggacctgcaggagaccttcagcgggatggatcaggtgcggctgatgaagaggattgcagaaatgacagatatcaaacccatcctgaggaagctcccgcgtatcaagaaacacttgttaaatggtgataatatgaggtgttcagtgaatgcgactcctcagcagatgcctcagacagaaaaagcggtcgaagacttccttagaagcatcggtcggagtaaaaaggaacggaggcctgtgcgcccacacacggtcgagaaacctgtgcccagcagctctggtggagatgcccacgttccccatggctcccaggtcattaggaagctggtcatggaacccaccttcaagccctggcagatgaagactcacttcctgatgcccttcccggtgaattacgtgggtgaatgcatccgaactgtcccctacacggacccagatcatgccagtcttaaaatccttgcacgtttgatgactgccaaattcttgcatacagaaattcgagaaaaaggcggtgcttatggtggaggcgcaaaactcagccacaatgggattttcaccctttactcttacagggacccaaatacaatagagacgctccagtcttttgggaaggctgtcgactgggctaagtctggaaaattcacacagcaagacatcgacgaagccaaactttctgtcttctcaaccgtagatgctcctgtcgctccttcagacaaaggaatggaccacttcttgtacggcctctcggatgagatgaagcaggcccacagagagcagctctttgctgtcagccacgacaagctcctggccgtgagcgatagatacctcggcactgggaagagcacacacggcctggccatcctcggacccgagaacccgaaaattgccaaggacccatcctggatcatccaatga

SEQ ID NO: 17 - DNA, 1830 bp, Homo sapiens
  source 1..1830: mol_type = other DNA
  unsure 1..1830: note = Transcript: ENST00000678844.1 IDE-230
atgcggtaccggctagcgtggcttctgcaccccgcactgcccagcaccttccgctcagtcctcggcgcccgcctgccgcctccggagcgcctgtgtggtttccaaaaaaagacttacagcaaaatgaataatccagccatcaagagaataggaaatcacattaccaagtctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaagttacgtgcagaaggacctcaagaatgggttttccaagagtgcaaggacttgaatgctgttgcttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagctatgagcaaactttggttcaaacaagatgataagttttttttgccgaaggcttgtctcaactttgaatttttcagctttgataaaattgttggcttttggctgggtgcgatagctcacacctgtaatcctaacattttgggaagccgaggcaggaagatcgcttga

SEQ ID NO: 18 - DNA, 1155 bp, Homo sapiens
  source 1..1155: mol_type = other DNA
atgcggtaccggctagcgtggcttctgcaccccgcactgcccagcaccttccgctcagtcctcggcgcccgcctgccgcctccggagcgcctgtgtggtttccaaaaaaagacttacagcaaaatgaataatccagccatcaagagaataggaaatcacattaccaagtctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattatga

SEQ ID NO: 19 - DNA, 1047 bp, synthetic construct
  source 1..1047: mol_type = other DNA
  unsure 1..1047: note = mRFP fused to fragment of the Huntingtin exon 1 with 74 glutamine repetitions
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgccaaatccggactcagatctatgaaggccttcgagtccctcaagtccttccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaacagccgccaccgccgccgccgccgccgccgcctcctcagcttcctcagccgccgaattctgcagtcgacggtaccgcgggcccgggatccaccggatctagataa

SEQ ID NO: 20 - protein, 1265 aa, Arabidopsis thaliana
  UNSURE 1..1265: note = Uniprot: A0A654G7G2 / Tair Locus: AT5G42390.1
MASSSSSIFTGVKFSPILAPFNSGDSRRSRYLKDSRNKVRFNPSSPRLTPHRVRVEAPSLIPYNGLWAAQPNSHKGRLKRNIVSGKEATGISLSQGRNFCLTCKRNQAGIRRALPSAFVDRTAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTAPLPAAIVACVPTKVHVDGVGESDFNISPDEIIESVKSGLLAPIEAEPELEVPKELISQSQLKELTLQRNPCFVPIPGSGLTKLHDKETGITQLRLSNGIAVNYKKSTTESRAGVMRLIVGGGRAAETSDSKGAVVVGVRTLSEGGRVGDFSREQVELFCVNHLINCSLESTEEFIAMEFRFTLRDNGMQAAFQLLHMVLERSVWLEDAFDRARQLYLSYFRSIPKSLERATAHKLMIAMLNGDERFVEPTPKSLQSLNLESVKDAVMSHFVGDNMEVSIVGDFSEEEIERCILDYLGTVKASHDSAKPPGSEPILFRQPTAGLQFQQVFLKDTDERACAYIAGPAPNRWGFTVDGDDLFQSVSKLPVAHDGLLKSEEQLLEGGDRELQKKLRAHPLFFGVTMGLLAEIINSRLFTTVRDSLGLTYDVSFELNLFDRLNLGWYVISVTSTPGKVYKAVDACKSVLRGLHSNQIAPRELDRAKRTLLMRHEAELKSNAYWLNLLAHLQASSVPRKELSCIKELVSLYEAASIEDIYLAYNQLRVDEDSLYSCIGIAGAQAGEEITVLSEEEEPEDVFSGVVPVGRGSSMTTRPTT

SEQ ID NO: 21 - protein, 1086 aa, Arabidopsis thaliana
  UNSURE 1..1086: note = Uniprot: A0A1P8BEG1 / Tair Locus: AT5G42390.2
MASSSSSIFTGVKFSPILAPFNSGDSRRSRYLKDSRNKVRFNPSSPRLTPHRVRVEAPSLIPYNGLWAAQPNSHKGRLKRNIVSGKEATGISLSQGRNFCLTCKRNQAGIRRALPSAFVDRTAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTAPLPAAIVACVPTKVHVDGVGESDFNISPDEIIESVKSGLLAPIEAEPELEVPKELISQSQLKELTLQRNPCFVPIPGSGLTKLHDKETGITQLRLSNGIAVNYKKSTTESRAGVMRLIVGGGRAAETSDSKGAVVVGVRTLSEGGRVGDFSREQVELFCVNHLINCSLESTEEFIAMEFRFTLRDNGMQAAFQLLHMVLERSVWLEDAFDRARQLYLSYFRSIPKSLERATAHKLMIAMLNGDERFVEPTPKSLQSLNLESVKDAVMSHFVGDNMEVSIVGDFSEEEIERCILDYLGTVKASHDSAKPPGSEPILFRQPTAGLQFQQVFLKDTDERACAYIAGPAPNRWGFTVDGDDLFQSVSKLPVAHDGLLKSEEQLLEGGDRELQKKLRAHPLFFGVTMGLLAEIINSRIC

SEQ ID NO: 22 - protein, 1212 aa, synthetic construct
  UNSURE 1..1212: note = Deletion of 54 aminoacids corresponding to the N-terminal chloroplast transit peptide. A methione was added as start codon
MVEAPSLIPYNGLWAAQPNSHKGRLKRNIVSGKEATGISLSQGRNFCLTCKRNQAGIRRALPSAFVDRTAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTAPLPAAIVACVPTKVHVDGVGESDFNISPDEIIESVKSGLLAPIEAEPELEVPKELISQSQLKELTLQRNPCFVPIPGSGLTKLHDKETGITQLRLSNGIAVNYKKSTTESRAGVMRLIVGGGRAAETSDSKGAVVVGVRTLSEGGRVGDFSREQVELFCVNHLINCSLESTEEFIAMEFRFTLRDNGMQAAFQLLHMVLERSVWLEDAFDRARQLYLSYFRSIPKSLERATAHKLMIAMLNGDERFVEPTPKSLQSLNLESVKDAVMSHFVGDNMEVSIVGDFSEEEIERCILDYLGTVKASHDSAKPPGSEPILFRQPTAGLQFQQVFLKDTDERACAYIAGPAPNRWGFTVDGDDLFQSVSKLPVAHDGLLKSEEQLLEGGDRELQKKLRAHPLFFGVTMGLLAEIINSRLFTTVRDSLGLTYDVSFELNLFDRLNLGWYVISVTSTPGKVYKAVDACKSVLRGLHSNQIAPRELDRAKRTLLMRHEAELKSNAYWLNLLAHLQASSVPRKELSCIKELVSLYEAASIEDIYLAYNQLRVDEDSLYSCIGIAGAQAGEEITVLSEEEEPEDVFSGVVPVGRGSSMTTRPTT

SEQ ID NO: 23 - protein, 1466 aa, synthetic construct
  UNSURE 1..1466: note = GFP was fused to the N-terminal of SPP. SPP has a deletion of 54 aminoacids corresponding to the N-terminal chloroplast transit peptide. No methione added as the start codon comes from GFP.
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKSGSGPDQTSLYKKAGWVEAPSLIPYNGLWAAQPNSHKGRLKRNIVSGKEATGISLSQGRNFCLTCKRNQAGIRRALPSAFVDRTAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTAPLPAAIVACVPTKVHVDGVGESDFNISPDEIIESVKSGLLAPIEAEPELEVPKELISQSQLKELTLQRNPCFVPIPGSGLTKLHDKETGITQLRLSNGIAVNYKKSTTESRAGVMRLIVGGGRAAETSDSKGAVVVGVRTLSEGGRVGDFSREQVELFCVNHLINCSLESTEEFIAMEFRFTLRDNGMQAAFQLLHMVLERSVWLEDAFDRARQLYLSYFRSIPKSLERATAHKLMIAMLNGDERFVEPTPKSLQSLNLESVKDAVMSHFVGDNMEVSIVGDFSEEEIERCILDYLGTVKASHDSAKPPGSEPILFRQPTAGLQFQQVFLKDTDERACAYIAGPAPNRWGFTVDGDDLFQSVSKLPVAHDGLLKSEEQLLEGGDRELQKKLRAHPLFFGVTMGLLAEIINSRLFTTVRDSLGLTYDVSFELNLFDRLNLGWYVISVTSTPGKVYKAVDACKSVLRGLHSNQIAPRELDRAKRTLLMRHEAELKSNAYWLNLLAHLQASSVPRKELSCIKELVSLYEAASIEDIYLAYNQLRVDEDSLYSCIGIAGAQAGEEITVLSEEEEPEDVFSGVVPVGRGSSMTTRPTT

SEQ ID NO: 24 - protein, 530 aa, synthetic construct
  PEPTIDE 1..530: note = Uniprot: Q5SXN9
MSGGAYPNIPLSSPLPGVPKPVFATVDGQEKFETKVTTLDNGLRVASQNKFGQFCTVGILINSGSRYEAKYLSGIAHFLEKLAFSSTARFDSKDEILLTLEKHGGICDCQTSRDTTMYAVSADSKGLDTVVALLADVVLQPRLTDEEVEMTRMAVQFELEDLNLRPDPEPLLTEMIHEAAYRENTVGLHRFCPTENVAKINREVLHSYLRNYYTPDRMVLAGVGVEHEHLVDCARKYLLGVQPAWGSAEAVDIDRSVAQYTGGIAKLERDMSNVSLGPTPIPELTHIMVGLESCSFLEEDFIPFAVLNMMMGGGGSFSAGGPGKGMFSRLYLNVLNRHHWMYNATSYHHSYEDTGLLCIHASADPRQVREMVEIITKEFILMGGTVDTVELERAKTQLTSMLMMNLESRPVIFEDVGRQVLATRSRKLPHELCTLIREYRRGSEGLPQASARLRGGRLALPQAVVGLWYVHHTQNLGPSPVVLSRGGQGQGRGVADLVSLPGPTMHLLPPWQVTPPSAPLGGAVTTAQSC

SEQ ID NO: 25 - protein, 434 aa, synthetic construct
  PEPTIDE 1..434: note = Uniprot: O75439 /Protein sequence AAC39915.1
METRVTCLESGLRVASEDSGLSTCTVGLWIDAGSRYENEKNNGTAHFLEHMAFKGTKKRSQLDLELEIENMGAHLNAYTSREQTVYYAKAFSKDLPRAVEILADIIQNSTLGEAEIERERGVILREMQEVETNLQEVVFDYLHATAYQNTALGRTILGPTENIKSISRKDLVDYITTHYKGPRIVLAAAGGVSHDELLDLAKFHFGDSLCTHKGEIPALPPCKFTGSEIRVRDDKMPLAHLAIAVEAVGWAHPDTICLMVANTLIGNWDRSFGGGMNLSSKLAQLTCHGNLCHSFQSFNTSYTDTGLWGLYMVCESSTVADMLHVVQKEWMRLCTSVTESEVARARNLLKTNMLLQLDGSTPICEDIGRQMLCYNRRIPIPELEARIDAVNAETIREVCTKYIYNRSPAIAAVGPIKQLPDFKQIRSNMCWLRD

SEQ ID NO: 26 - protein, 489 aa, Homo sapiens
  PEPTIDE 1..489: note = Uniprot: O75439 /Protein sequence AAC39915.1
MAAAAARVVLSSAARRRLWGFSESLLIRGAAGRSLYFGENRLRSTQAATQVVLNVPETRVTCLESGLRVASEDSGLSTCTVGLWIDAGSRYENEKNNGTAHFLEHMAFKGTKKRSQLDLELEIENMGAHLNAYTSREQTVYYAKAFSKDLPRAVEILADIIQNSTLGEAEIERERGVILREMQEVETNLQEVVFDYLHATAYQNTALGRTILGPTENIKSISRKDLVDYITTHYKGPRIVLAAAGGVSHDELLDLAKFHFGDSLCTHKGEIPALPPCKFTGSEIRVRDDKMPLAHLAIAVEAVGWAHPDTICLMVANTLIGNWDRSFGGGMNLSSKLAQLTCHGNLCHSFQSFNTSYTDTGLWGLYMVCESSTVADMLHVVQKEWMRLCTSVTESEVARARNLLKTNMLLQLDGSTPICEDIGRQMLCYNRRIPIPELEARIDAVNAETIREVCTKYIYNRSPAIAAVGPIKQLPDFKQIRSNMCWLRD

SEQ ID NO: 27 - protein, 563 aa, Homo sapiens
  PEPTIDE 1..563: note = Uniprot: Q5SXN9
MAAVVLAATRLLRGSGSWGCSRLRFGPPAYRRFSSGGAYPNIPLSSPLPGVPKPVFATVDGQEKFETKVTTLDNGLRVASQNKFGQFCTVGILINSGSRYEAKYLSGIAHFLEKLAFSSTARFDSKDEILLTLEKHGGICDCQTSRDTTMYAVSADSKGLDTVVALLADVVLQPRLTDEEVEMTRMAVQFELEDLNLRPDPEPLLTEMIHEAAYRENTVGLHRFCPTENVAKINREVLHSYLRNYYTPDRMVLAGVGVEHEHLVDCARKYLLGVQPAWGSAEAVDIDRSVAQYTGGIAKLERDMSNVSLGPTPIPELTHIMVGLESCSFLEEDFIPFAVLNMMMGGGGSFSAGGPGKGMFSRLYLNVLNRHHWMYNATSYHHSYEDTGLLCIHASADPRQVREMVEIITKEFILMGGTVDTVELERAKTQLTSMLMMNLESRPVIFEDVGRQVLATRSRKLPHELCTLIREYRRGSEGLPQASARLRGGRLALPQAVVGLWYVHHTQNLGPSPVVLSRGGQGQGRGVADLVSLPGPTMHLLPPWQVTPPSAPLGGAVTTAQSC

SEQ ID NO: 28 - protein, 17 aa, synthetic construct
GGGGSFSAGGPGKGMFS

SEQ ID NO: 29 - protein, 586 aa, synthetic construct
  UNSURE 1..586: note = Truncated version of SPP (AT5G42390.1) residues 122 to 707
TAFSLSRSSLTSSLRKHSQIVNATLGPDEPHAAGTAWPDGIVAERQDLDLLPPEIDSAELEAFLGCELPSHPKLHRGQLKNGLRYLILPNKVPPNRFEAHMEVHVGSIDEEEDEQGIAHMIEHVAFLGSKKREKLLGTGARSNAYTDFHHTVFHIHSPTHTKDSEDDLFPSVLDALNEIAFHPKFLSSRVEKERRAILSELQMMNTIEYRVDCQLLQHLHSENKLGRRFPIGLEEQIKKWDVDKIRKFHERWYFPANATLYIVGDIDNIPRIVHNIEAVFGKNGLDNESTPSSPSPGAFGAMANFLVPKLPAGLGGTFSNEKTNTADQSKMIKRERHAIRPPVEHNWSLPGTSVDLKPPQIFKHELLQNFAINMFCKIPVSKVQTFGDLRNVLMKRIFLSALHFRINTRYKSSNPPFTSVELDHSDSGREGCTVTTLTVTAEPQNWQNAVKVAVQEVRRLKEFGVTRGELTRYMDALLKDSEHLAAMIDNVSSVDNLDFIMESDALSHTVMDQTQGHETLVAVAGTVTLEEVNTVGAKVLEFISDFGRPTAPLPAAIVACVPTKVHVDGVGESDFNISPDEIIESV

SEQ ID NO: 30 - protein, 260 aa, synthetic construct
  UNSURE 1..260: note = Truncated version of SPP (AT5G42390.1) residues 1004 to 1263
DTDERACAYIAGPAPNRWGFTVDGDDLFQSVSKLPVAHDGLLKSEEQLLEGGDRELQKKLRAHPLFFGVTMGLLAEIINSRLFTTVRDSLGLTYDVSFELNLFDRLNLGWYVISVTSTPGKVYKAVDACKSVLRGLHSNQIAPRELDRAKRTLLMRHEAELKSNAYWLNLLAHLQASSVPRKELSCIKELVSLYEAASIEDIYLAYNQLRVDEDSLYSCIGIAGAQAGEEITVLSEEEEPEDVFSGVVPVGRGSSMTTRP

SEQ ID NO: 31 - protein, 82 aa, synthetic construct
  UNSURE 1..82: note = Zn2+-binding region (HxxEHx76E motif ) from SPP (AT5G42390.1)
HMIEHVAFLGSKKREKLLGTGARSNAYTDFHHTVFHIHSPTHTKDSEDDLFPSVLDALNEIAFHPKFLSSRVEKERRAILSE

SEQ ID NO: 32 - protein, 81 aa, synthetic construct
  UNSURE 1..81: note = Zn2+-binding region (HxxEHx76E motif ) from HUMAN Mitochondrial-processing peptidase subunit beta
HFLEHMAFKGTKKRSQLDLELEIENMGAHLNAYTSREQTVYYAKAFSKDLPRAVEILADIIQNSTLGEAEIERERGVILRE

SEQ ID NO: 33 - protein, 948 aa, Homo sapiens [sequence omitted]

SEQ ID NO: 34 - protein, 747 aa, Homo sapiens [sequence omitted]

SEQ ID NO: 35 - protein, 1021 aa, Homo sapiens
  PEPTIDE 1..1021: note = Uniprot: A0A7I2YQT2
MWRCGGRQGLCVLRRLSGGHAHHRAWRWNSNRACERALQYKLGDKIHGFTVNQVTSVPELFLTAVKLTHDDTGARYLHLAREDTNNLFSVQFRTTPMDSTGVPHILEHTVLCGSQKYPCRDPFFKMLNRSLSTFMNAFTGLSESPLGVFGCHLFPMFTRAGFLEGWRLEHENPSDPQTPLVFKGVVFNEMKGAFTDNERIFSQHLQNRLLPDHTYSVVSGGDPLCIPELTWEQLKQFHATHYHPSNARFFTYGNFPLEQHLKQIHEEALSKFQKIEPSTVVPAQTPWDKPREFQITCGPDSFATDPSKQTTISVSFLLPDITDTFEAFTLSLLSSLLTSGPNSPFYKALIESGLGTDFSPDVGYNGYTREAYFSVGLQGIAEKDIETVRSLIDRTIDEVVEKGFEDDRIEALLHKIEIQMKHQSTSFGLMLTSYIASCWNHDGDPVELLKLGNQLAKFRQCLQENPKFLQEKVKQYFKNNQHKLTLSMRPDDKYHEKQAQVEATKLKQKVEALSPGDRQQIYEKGLELRSQQSKPQDASCLPALKVSDIEPTIPVTELDVVLTAGDIPVQYCAQPTNGMVYFRAFSSLNTLPEELRPYVPLFCSVLTKLGCGLLDYREQAQQIELKTGGMSASPHVLPDDSHMDTYEQGVLFSSLCLDRNLPDMMQLWSEIFNNPCFEEEEHFKVLVKMTAQELANGIPDSGHLYASIRAGRTLTPAGDLQETFSGMDQVRLMKRIAEMTDIKPILRKLPRIKKHLLNGDNMRCSVNATPQQMPQTEKAVEDFLRSIGRSKKERRPVRPHTVEKPVPSSSGGDAHVPHGSQVIRKLVMEPTFKPWQMKTHFLMPFPVNYVGECIRTVPYTDPDHASLKILARLMTAKFLHTEIREKGGAYGGGAKLSHNGIFTLYSYRDPNTIETLQSFGKAVDWAKSGKFTQQDIDEAKLSVFSTVDAPVAPSDKGMDHFLYGLSDEMKQAHREQLFAVSHDKLLAVSDRYLGTGKSTHGLAILGPENPKIAKDPSWIIQ

SEQ ID NO: 36 - protein, 609 aa, Homo sapiens
  PEPTIDE 1..609: note = Uniprot: A0A7I2V612
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSFDKIVGFWLGAIAHTCNPNILGSRGRKIA

SEQ ID NO: 37 - protein, 384 aa, Homo sapiens
  PEPTIDE 1..384: note = Uniprot: A0A7I2V634
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGL

SEQ ID NO: 38 - protein, 348 aa, synthetic construct
  UNSURE 1..348: note = mRFP fused to fragment of the Huntingtin exon 1 with 74 glutamine repetitions
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGAKSGLRSMKAFESLKSFQQQQQQQQQQQQQQQQQQQQQQQQQQQQQQQQQQQQQQQQQQQQQQQQQQQQQQQQQQQQQQQQQQQQQQQQQQPPPPPPPPPPPQLPQPPNSAVDGTAGPGSTGSR

SEQ ID NO: 39 - DNA, 53 bp, synthetic construct
  source 1..53: mol_type = other DNA
  primer_bind 1..53: note = Primers used for plant expression plasmid
ggggacaagtttgtacaaaaaagcaggctggatggtgagcaagggcgaggagc

SEQ ID NO: 40 - DNA, 54 bp, synthetic construct
  source 1..54: mol_type = other DNA
  primer_bind 1..54: note = Primers used for plant expression plasmid
ggggaccactttgtacaagaaagctgggtcttatctagatccggtggatcccgg

SEQ ID NO: 41 - DNA, 53 bp, synthetic construct; identical to SEQ ID NO: 39
  source 1..53: mol_type = other DNA
  primer_bind 1..53: note = Primers used for plant expression plasmid

SEQ ID NO: 42 - DNA, 34 bp, synthetic construct
  source 1..34: mol_type = other DNA
  primer_bind 1..34: note = Primers used for C. elegans expression plasmid
ttggctagcatggtagaagccccctcactcatac

SEQ ID NO: 43 - DNA, 33 bp, synthetic construct
  source 1..33: mol_type = other DNA
  primer_bind 1..33: note = Primers used for C. elegans expression plasmid
ccagcggccgcttatgtagtcgggcgggttgtc

SEQ ID NO: 44 - DNA, 22 bp, synthetic construct
  source 1..22: mol_type = other DNA
  primer_bind 1..22: note = Primers for qPCR
ctggaggttttgaggctggtat

SEQ ID NO: 45 - DNA, 21 bp, synthetic construct
  source 1..21: mol_type = other DNA
  primer_bind 1..21: note = Primers for qPCR
ccaagggtgaaagcaagaaga

SEQ ID NO: 46 - DNA, 20 bp, synthetic construct
  source 1..20: mol_type = other DNA
  primer_bind 1..20: note = Primers for qPCR
taacgtggccaaaatgatgc

SEQ ID NO: 47 - DNA, 20 bp, synthetic construct
  source 1..20: mol_type = other DNA
  primer_bind 1..20: note = Primers for qPCR
gttctccacaaccgcttggt

SEQ ID NO: 48 - DNA, 20 bp, synthetic construct
  source 1..20: mol_type = other DNA
  primer_bind 1..20: note = Primers for qPCR
aaggaaacagaaccacgcca

SEQ ID NO: 49 - DNA, 20 bp, synthetic construct
  source 1..20: mol_type = other DNA
  primer_bind 1..20: note = Primers for qPCR
tgtcagagaaacgacgaccg

SEQ ID NO: 50 - DNA, 23 bp, synthetic construct
  source 1..23: mol_type = other DNA
  primer_bind 1..23: note = Primers for qPCR
gcagaagattgagaaggcgattg

SEQ ID NO: 51 - DNA, 23 bp, synthetic construct
  source 1..23: mol_type = other DNA
  primer_bind 1..23: note = Primers for qPCR
cgctctaatccacctcttcgatc

SEQ ID NO: 52 - DNA, 22 bp, synthetic construct
  source 1..22: mol_type = other DNA
  primer_bind 1..22: note = Primers for qPCR
aagctcgatggacagcctgaac

SEQ ID NO: 53 - DNA, 24 bp, synthetic construct
  source 1..24: mol_type = other DNA
  primer_bind 1..24: note = Primers for qPCR
tcccaagttgttcaccaaatctgc

SEQ ID NO: 54 - DNA, 20 bp, synthetic construct
  source 1..20: mol_type = other DNA
  primer_bind 1..20: note = Primers for qPCR
gaggagttgctttggcagtc

SEQ ID NO: 55 - DNA, 20 bp, synthetic construct
  source 1..20: mol_type = other DNA
  primer_bind 1..20: note = Primers for qPCR
cagcgcctgcatctatgtaa